(19) United States
(12) Patent Application Publication
(10) Pub. No.: US 2021/0163411 A1
(43) Pub. Date: Jun. 3, 2021

(54) COMPOUND HAVING TRIARYLAMINE STRUCTURE AS CORE, AND PREPARATION METHOD THEREFOR

(71) Applicant: JIANGSU SUNERA TECHNOLOGY CO., LTD., Jiangsu (CN)

(72) Inventors: Chong Li, Jiangsu (CN); Xiaoqing Zhang, Jiangsu (CN); Zhaochao Zhang, Jiangsu (CN); Sijie Zhao, Jiangsu (CN)

(21) Appl. No.: 15/734,590

(22) PCT Filed: Jun. 5, 2019
(86) PCT No.: PCT/CN2019/090085

§ 371 Date: Dec. 3, 2020

(30) Foreign Application Priority Data
Jun. 7, 2018 (CN) 201810580844.6

(51) Int. Cl. C07D 209/86; C07D 491/048; C07D 519/00; H01L 51/00

(52) U.S. Cl. CPC C07D 209/86; C07D 491/048; C07D 519/00; H01L 51/0058; H01L 51/5096; H01L 51/0073; H01L 51/0059; H01L 2251/552; H01L 51/0072

(57) ABSTRACT

A compound having a triarylamine structure as a core, a preparation method therefor, and an application thereof. The compound has a structure represented by general formula (1). The compound has high glass transition temperature and molecular thermal stability, has suitable HOMO and LIMO energy levels, and has high mobility. By means of device structure optimization, the photoelectric properties of an OLED device can be effectively improved and the life of the OLED device can be effectively prolonged.

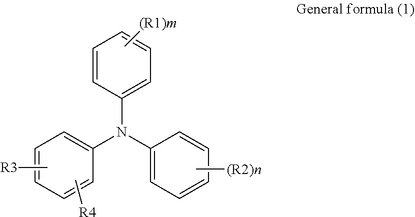

### TECHNICAL FIELD

The present invention relates to the technical field of organic light-emitting diode materials, and in particular relates to a compound containing triarylamine in its structure and a preparation method thereof.

### BACKGROUND ART

Organic light-emitting diode (OLED) technology can be used for manufacturing not only new display products but also new lighting products, which are expected to replace existing liquid crystal displays and fluorescent lighting, having a broad application prospect.

An organic light-emitting device has a sandwich-like structure, which comprises electrode material film layers and organic functional materials sandwiched between different electrode film layers; various functional materials are superimposed on each other according to their uses to form the organic light-emitting device. As a current device, the OLED device produces electroluminescence when a voltage is applied to the electrodes at both ends of the OLED device, and the positive charges and the negative charges produced in the functional material film of the organic layer under the action of the electric field are recombined in the luminescent layer.

Currently, OLED display technology has been applied in smart phones, tablet computers and other fields, and will further expand to large-size applications such as television. However, in order to meet the actual product application requirements, the properties of OLED, such as luminous efficiency and service life, need to be further improved.

Research on improving the properties of an organic light-emitting device includes reducing the driving voltage of the device, improving the luminous efficiency of the device, and increasing the service life of the device. In order to achieve continuous improvement in the properties of OLED, there is a need not only for innovating the structure and preparation process of OLED, but also for continuously researching and innovating OLED photoelectric functional materials to create OLED functional materials with higher performance.

OLED photoelectric functional materials used in OLED can be divided, according to their use, into two categories: charge injection-transporting materials and light-emitting materials. Further, charge injection-transporting materials can be divided into electron injection-transporting materials, electron-blocking materials, hole injection-transporting materials, and hole blocking materials. Light-emitting materials can be further divided into host light-emitting materials and doping materials.

In order to make a high-performance organic light-emitting device, it is required that its various organic functional materials have good photoelectric properties. For example, the charge transporting materials are required to have, among others, good carrier mobility and high glass transition temperature. The host materials of the light-emitting layer are required to have good bipolarity, proper HOMO/LUMO energy level, and other properties.

The OLED photoelectric functional material film layers which constitute OLED have a structure of at least two or more layers. The structure of OLED used in industry includes a variety of film layers such as a hole injection layer, a hole transporting layer, an electron blocking layer, a light-emitting layer, a hole blocking layer, an electron transporting layer and an electron injection layer. That is, the photoelectric functional materials used in OLED include at least hole injection materials, hole transporting materials, light-emitting materials, electron transporting materials and the like. The types of materials and the forms of combinations are characterized by richness and diversity. Furthermore, for the combination of OLED with different structures, the photoelectric functional materials used have relatively high selectivity, and the same material in structurally different devices can display completely different performances.

Therefore, in view of the current requirements for industrial application of OLED, the different functional film layers of OLED and the photoelectric characteristics of the devices, it is necessary to select more suitable high-performance OLED functional materials or material combinations to achieve the comprehensive characteristics of high efficiency, long life and low voltage of the device. Regarding the actual requirements of the current OLED display lighting industry, the current development of OLED materials is largely insufficient, lagging behind the requirements of panel manufacturers. It is particularly important for material companies to develop organic functional materials with higher performance.

### SUMMARY OF THE INVENTION

With respect to the aforementioned problems existed in the prior art, the present inventors have provided a compound with a core structure of triarylamine and a preparation method thereof.

The technical solutions of the present invention are as follows.

The present invention provides a compound with a core structure of triarylamine, characterized in that the compound has a structure represented by general formula (1):

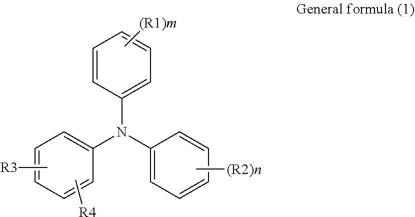

wherein

m and n each independently represents an integer of 1, 2 or 3;

R₁, R₂, R₃ and R₄ each independently represents substituted or unsubstituted phenyl, substituted or unsubstituted biphenyl, substituted or unsubstituted naphthyl, or a structure represented by general formula (2) or general formula (3), with R₃ and R₄ positioned ortho to each other;

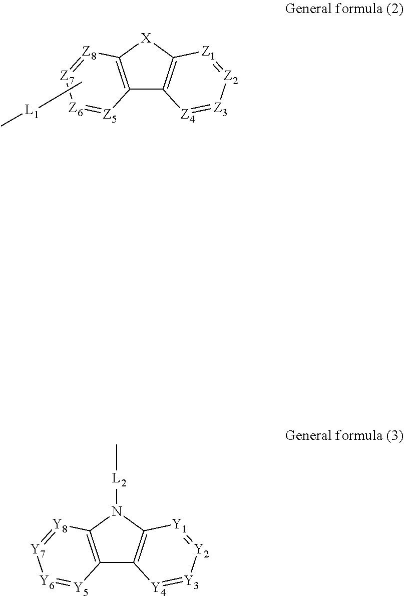

in the general formula (2) and the general formula (3), L₁ and L₂ each independently represents a single bond, substituted or unsubstituted phenylene, substituted or unsubstituted naphthylene, or substituted or unsubstituted biphenylene;

in the general formula (2), X represents —O—, —S—, —C(R₅)(R₆)— or —N(R₇)—;

Z₁-Z₈ each independently represents CH or N, with at most 4 N;

in the general formula (2), Z₅, Z₆, Z₇ or Z₈ to which L₁ is bonded represents a carbon atom;

in the general formula (3), Y₁-Y₈ each independently represents CH or N, with at most 4 N;

R₅ to R₇ each independently represents one of C₁₋₂₀ alkyl, C₆₋₃₀ aryl, and substituted or unsubstituted 5- to 30-membered heteroaryl containing one or more heteroatoms, wherein R₅ and R₆ together with the atom to which they are bonded may form a 5-membered to 30-membered alicyclic or aromatic ring;

the substituent is halogen, cyano, C₁₋₁₀ alkyl or C₆₋₂₀ aryl.

In a preferred embodiment, R₅ to R₇ each independently represents methyl, ethyl, propyl, isopropyl, butyl, tert-butyl, pentyl, phenyl, biphenyl, terphenyl, naphthyl, pyridyl or furyl.

In a preferred embodiment, the structure of the general formula (2) is represented by any one of

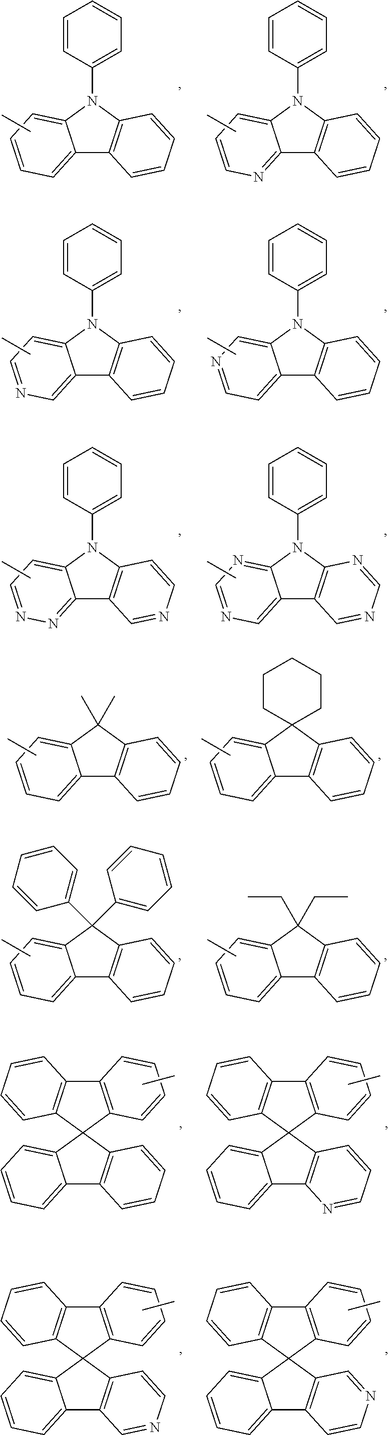

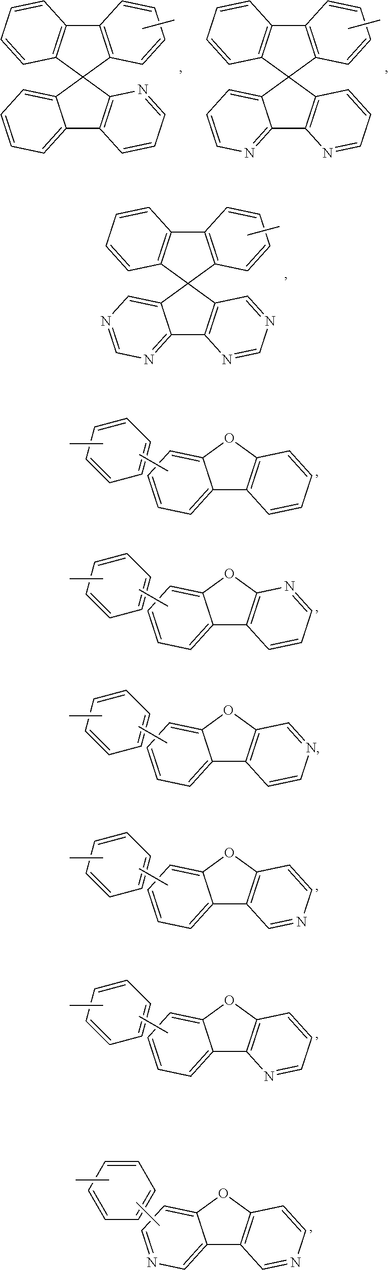

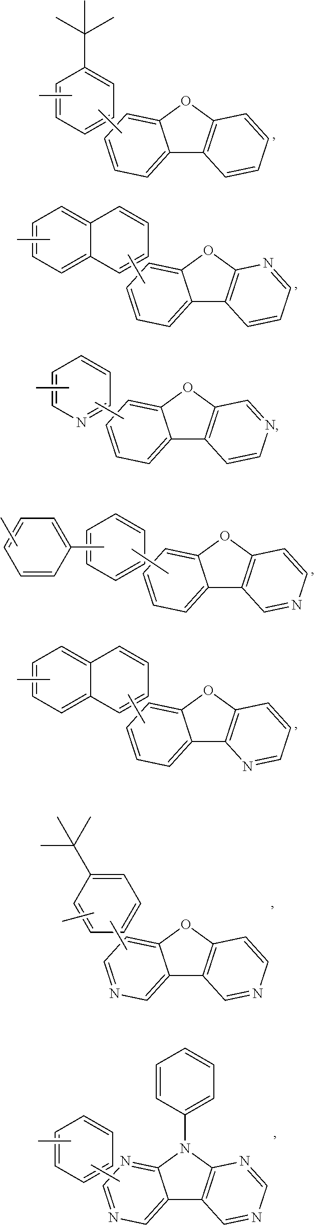

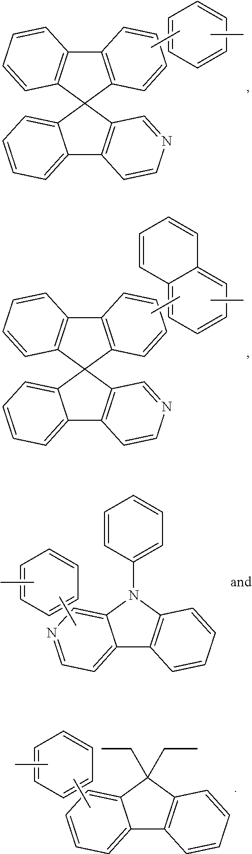

In a preferred embodiment, the structure of the general formula (3) is represented by any one of

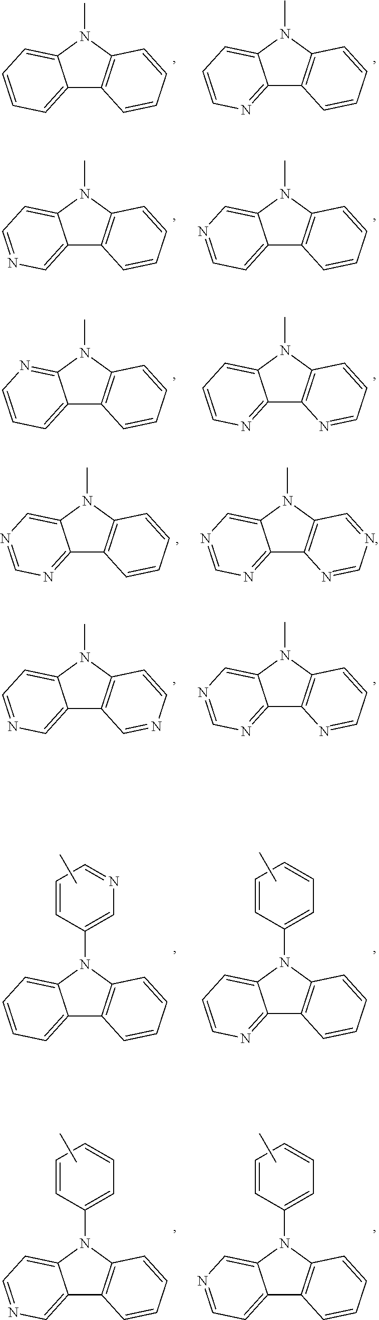

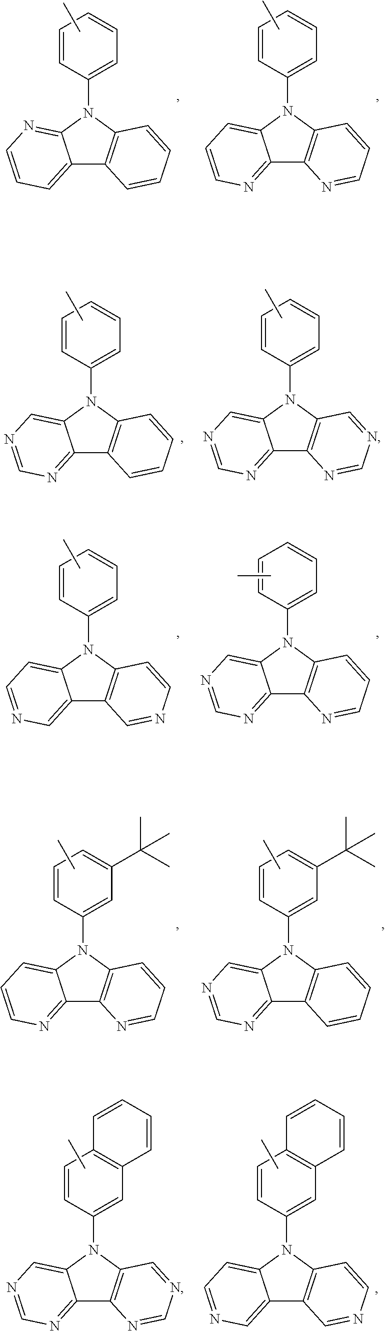

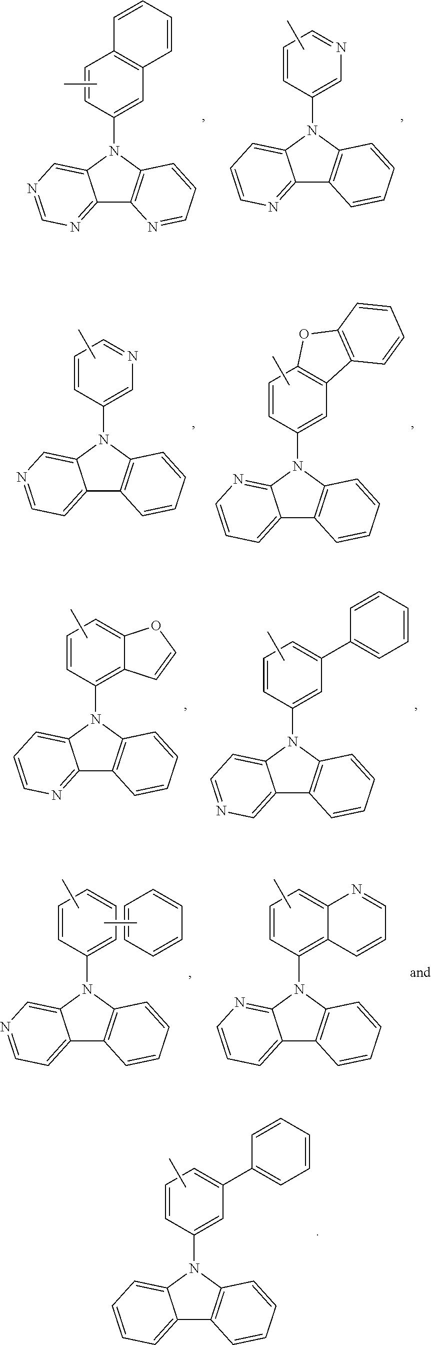

The compound with a core structure of triarylamine has a preferred specific structure of any one of

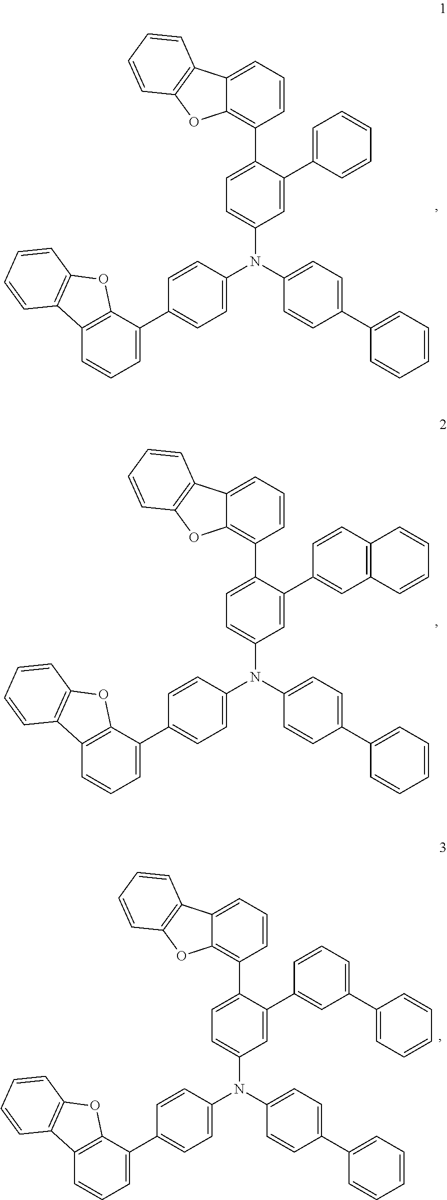 [SMILES: c1ccc(-c2ccc(N(c3ccc(-c4cccc5c4oc4ccccc45)cc3)c3ccc(-c4cccc5c4oc4ccccc45)c(-c4ccccc4)c3)cc2)cc1 ; c1ccc(-c2ccc(N(c3ccc(-c4cccc5c4oc4ccccc45)cc3)c3ccc(-c4cccc5c4oc4ccccc45)c(-c4ccc5ccccc5c4)c3)cc2)cc1 ; c1ccc(-c2ccc(N(c3ccc(-c4cccc5c4oc4ccccc45)cc3)c3ccc(-c4cccc5c4oc4ccccc45)c(-c4cccc(-c5ccccc5)c4)c3)cc2)cc1]

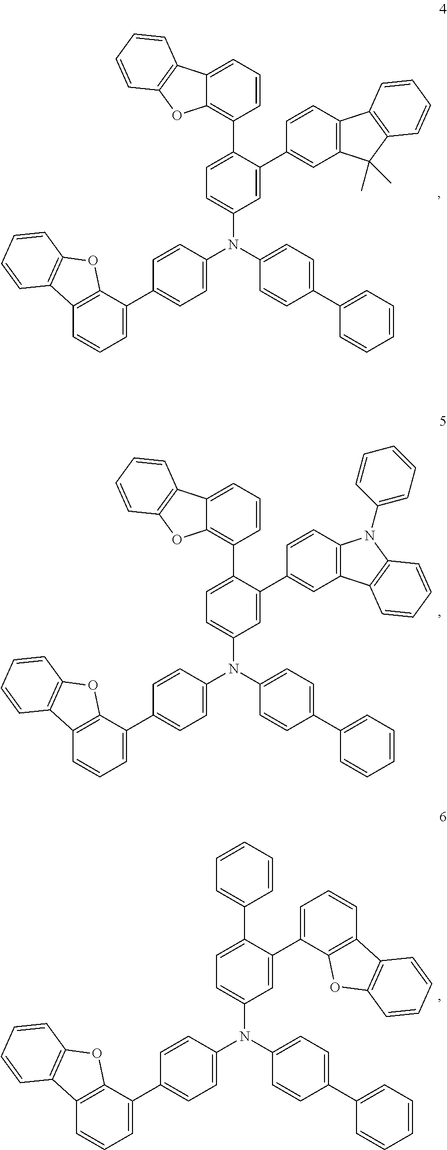 [SMILES: CC1(C)c2ccccc2-c2ccc(-c3cc(N(c4ccc(-c5ccccc5)cc4)c4ccc(-c5cccc6c5oc5ccccc56)cc4)ccc3-c3cccc4c3oc3ccccc34)cc21 ; c1ccc(-c2ccc(N(c3ccc(-c4cccc5c4oc4ccccc45)cc3)c3ccc(-c4cccc5c4oc4ccccc45)c(-c4ccc5c(c4)c4ccccc4n5-c4ccccc4)c3)cc2)cc1 ; c1ccc(-c2ccc(N(c3ccc(-c4cccc5c4oc4ccccc45)cc3)c3ccc(-c4ccccc4)c(-c4cccc5c4oc4ccccc45)c3)cc2)cc1]

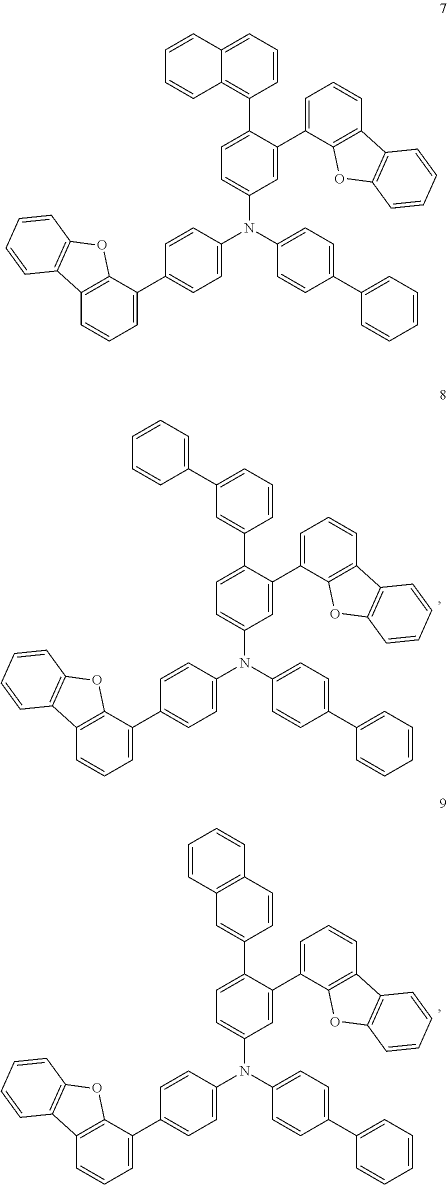 [SMILES: c1ccc(-c2ccc(N(c3ccc(-c4cccc5c4oc4ccccc45)cc3)c3ccc(-c4cccc5ccccc45)c(-c4cccc5c4oc4ccccc45)c3)cc2)cc1 ; c1ccc(-c2ccc(N(c3ccc(-c4cccc5c4oc4ccccc45)cc3)c3ccc(-c4cccc(-c5ccccc5)c4)c(-c4cccc5c4oc4ccccc45)c3)cc2)cc1 ; c1ccc(-c2ccc(N(c3ccc(-c4cccc5c4oc4ccccc45)cc3)c3ccc(-c4ccc5ccccc5c4)c(-c4cccc5c4oc4ccccc45)c3)cc2)cc1]

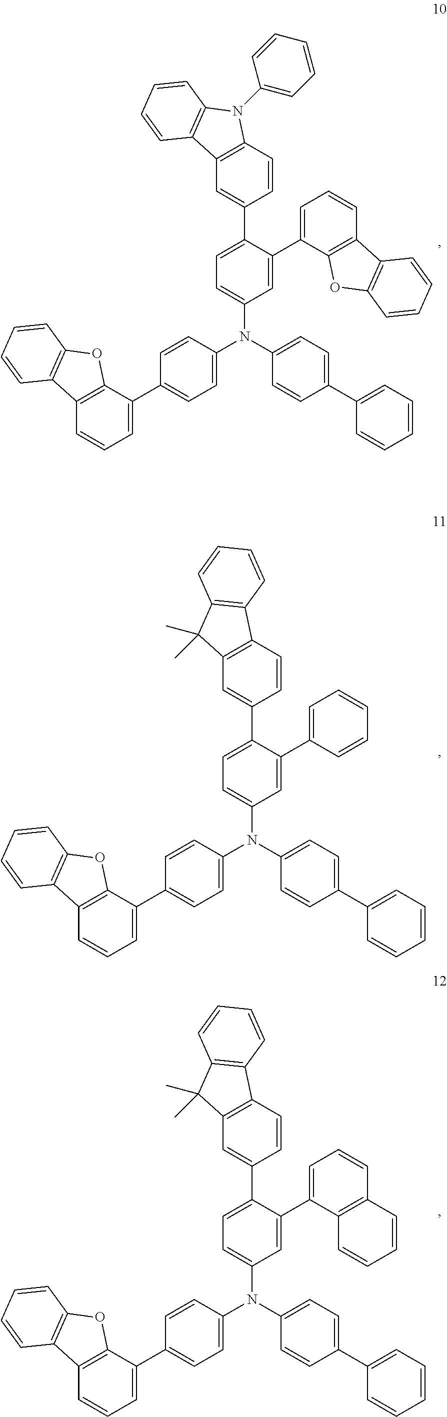 [SMILES: c1ccc(-c2ccc(N(c3ccc(-c4cccc5c4oc4ccccc45)cc3)c3ccc(-c4ccc5c(c4)c4ccccc4n5-c4ccccc4)c(-c4cccc5c4oc4ccccc45)c3)cc2)cc1 ; CC1(C)c2ccccc2-c2ccc(-c3ccc(N(c4ccc(-c5ccccc5)cc4)c4ccc(-c5cccc6c5oc5ccccc56)cc4)cc3-c3ccccc3)cc21 ; CC1(C)c2ccccc2-c2ccc(-c3ccc(N(c4ccc(-c5ccccc5)cc4)c4ccc(-c5cccc6c5oc5ccccc56)cc4)cc3-c3cccc4ccccc34)cc21]

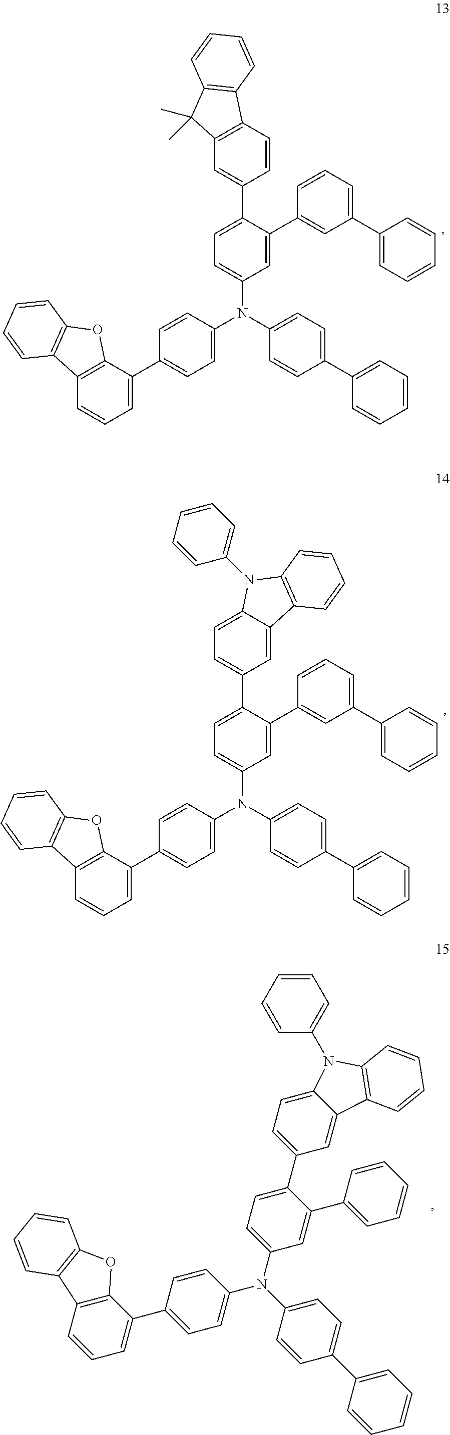 [SMILES: CC1(C)c2ccccc2-c2ccc(-c3ccc(N(c4ccc(-c5ccccc5)cc4)c4ccc(-c5cccc6c5oc5ccccc56)cc4)cc3-c3cccc(-c4ccccc4)c3)cc21 ; c1ccc(-c2ccc(N(c3ccc(-c4cccc5c4oc4ccccc45)cc3)c3ccc(-c4ccc5c(c4)c4ccccc4n5-c4ccccc4)c(-c4cccc(-c5ccccc5)c4)c3)cc2)cc1 ; c1ccc(-c2ccc(N(c3ccc(-c4cccc5c4oc4ccccc45)cc3)c3ccc(-c4ccc5c(c4)c4ccccc4n5-c4ccccc4)c(-c4ccccc4)c3)cc2)cc1]

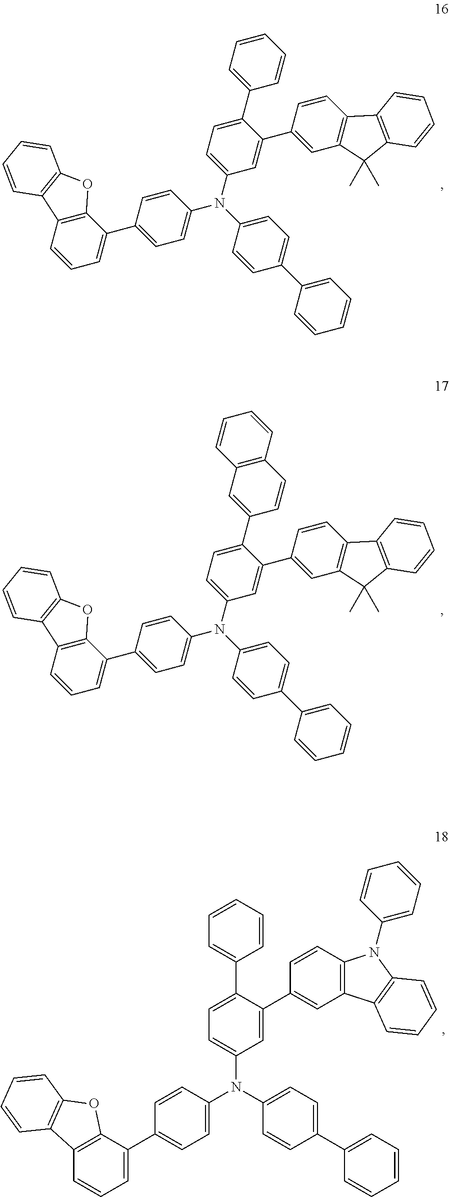 [SMILES: CC1(C)c2ccccc2-c2ccc(-c3cc(N(c4ccc(-c5ccccc5)cc4)c4ccc(-c5cccc6c5oc5ccccc56)cc4)ccc3-c3ccccc3)cc21 ; CC1(C)c2ccccc2-c2ccc(-c3cc(N(c4ccc(-c5ccccc5)cc4)c4ccc(-c5cccc6c5oc5ccccc56)cc4)ccc3-c3ccc4ccccc4c3)cc21 ; c1ccc(-c2ccc(N(c3ccc(-c4cccc5c4oc4ccccc45)cc3)c3ccc(-c4ccccc4)c(-c4ccc5c(c4)c4ccccc4n5-c4ccccc4)c3)cc2)cc1]

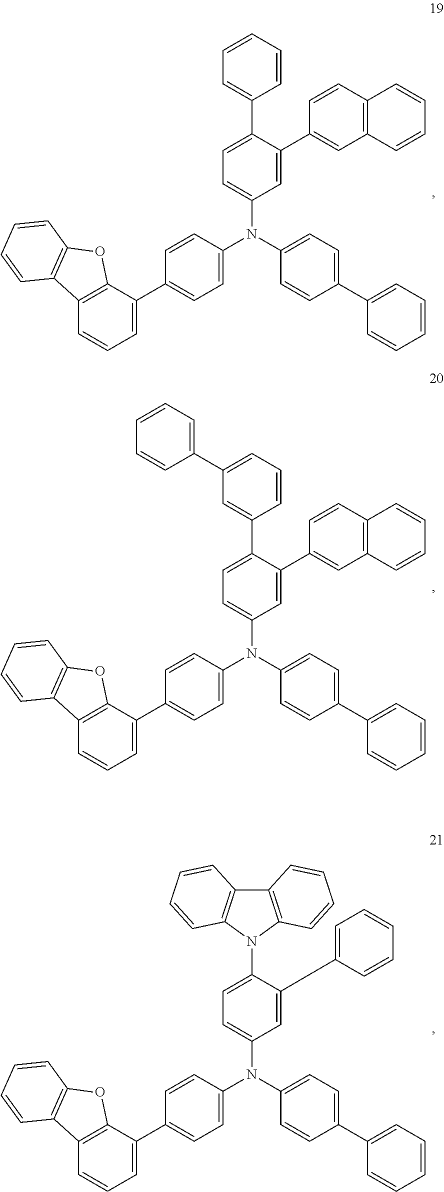 [SMILES: c1ccc(-c2ccc(N(c3ccc(-c4cccc5c4oc4ccccc45)cc3)c3ccc(-c4ccccc4)c(-c4ccc5ccccc5c4)c3)cc2)cc1 ; c1ccc(-c2ccc(N(c3ccc(-c4cccc5c4oc4ccccc45)cc3)c3ccc(-c4cccc(-c5ccccc5)c4)c(-c4ccc5ccccc5c4)c3)cc2)cc1 ; c1ccc(-c2ccc(N(c3ccc(-c4cccc5c4oc4ccccc45)cc3)c3ccc(-n4c5ccccc5c5ccccc54)c(-c4ccccc4)c3)cc2)cc1]

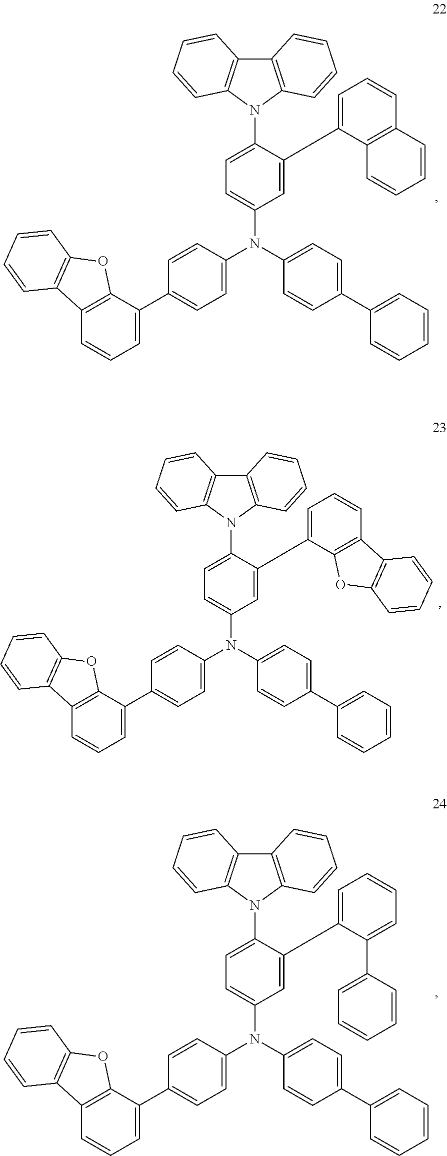 [SMILES: c1ccc(-c2ccc(N(c3ccc(-c4cccc5c4oc4ccccc45)cc3)c3ccc(-n4c5ccccc5c5ccccc54)c(-c4cccc5ccccc45)c3)cc2)cc1 ; c1ccc(-c2ccc(N(c3ccc(-c4cccc5c4oc4ccccc45)cc3)c3ccc(-n4c5ccccc5c5ccccc54)c(-c4cccc5c4oc4ccccc45)c3)cc2)cc1 ; c1ccc(-c2ccc(N(c3ccc(-c4cccc5c4oc4ccccc45)cc3)c3ccc(-n4c5ccccc5c5ccccc54)c(-c4ccccc4-c4ccccc4)c3)cc2)cc1]

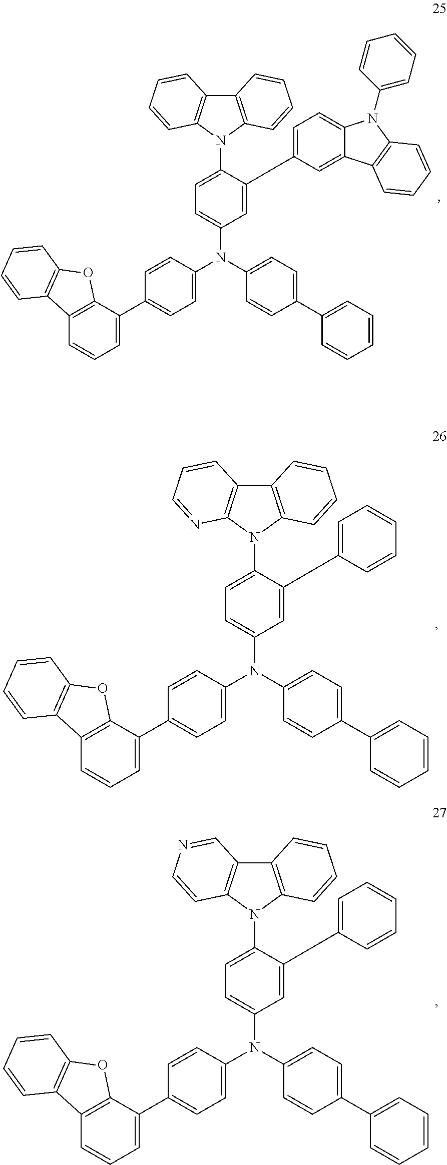 [SMILES: c1ccc(-c2ccc(N(c3ccc(-c4cccc5c4oc4ccccc45)cc3)c3ccc(-n4c5ccccc5c5ccccc54)c(-c4ccc5c(c4)c4ccccc4n5-c4ccccc4)c3)cc2)cc1 ; c1ccc(-c2ccc(N(c3ccc(-c4cccc5c4oc4ccccc45)cc3)c3ccc(-n4c5ccccc5c5cccnc54)c(-c4ccccc4)c3)cc2)cc1 ; c1ccc(-c2ccc(N(c3ccc(-c4cccc5c4oc4ccccc45)cc3)c3ccc(-n4c5ccccc5c5cnccc54)c(-c4ccccc4)c3)cc2)cc1]

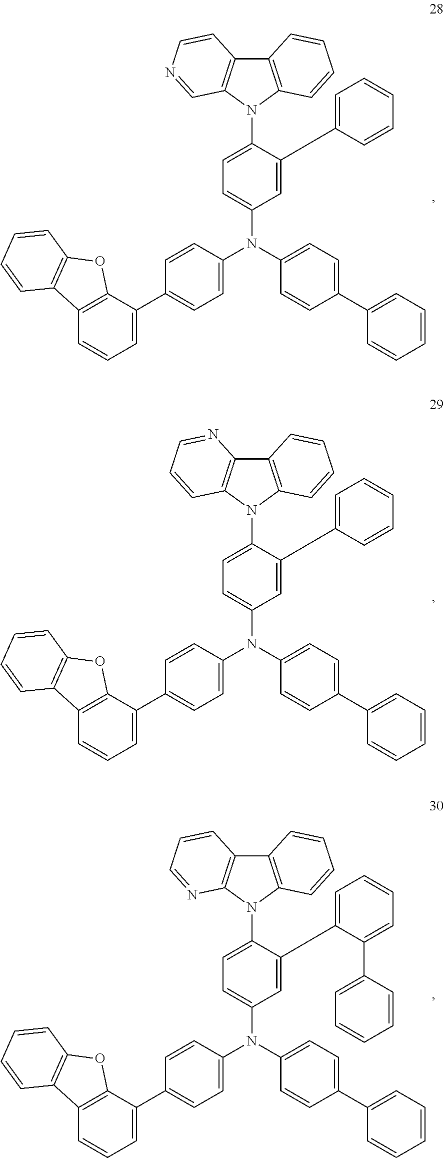 [SMILES: c1ccc(-c2ccc(N(c3ccc(-c4cccc5c4oc4ccccc45)cc3)c3ccc(-n4c5ccccc5c5ccncc54)c(-c4ccccc4)c3)cc2)cc1 ; c1ccc(-c2ccc(N(c3ccc(-c4cccc5c4oc4ccccc45)cc3)c3ccc(-n4c5ccccc5c5ncccc54)c(-c4ccccc4)c3)cc2)cc1 ; c1ccc(-c2ccc(N(c3ccc(-c4cccc5c4oc4ccccc45)cc3)c3ccc(-n4c5ccccc5c5cccnc54)c(-c4ccccc4-c4ccccc4)c3)cc2)cc1]

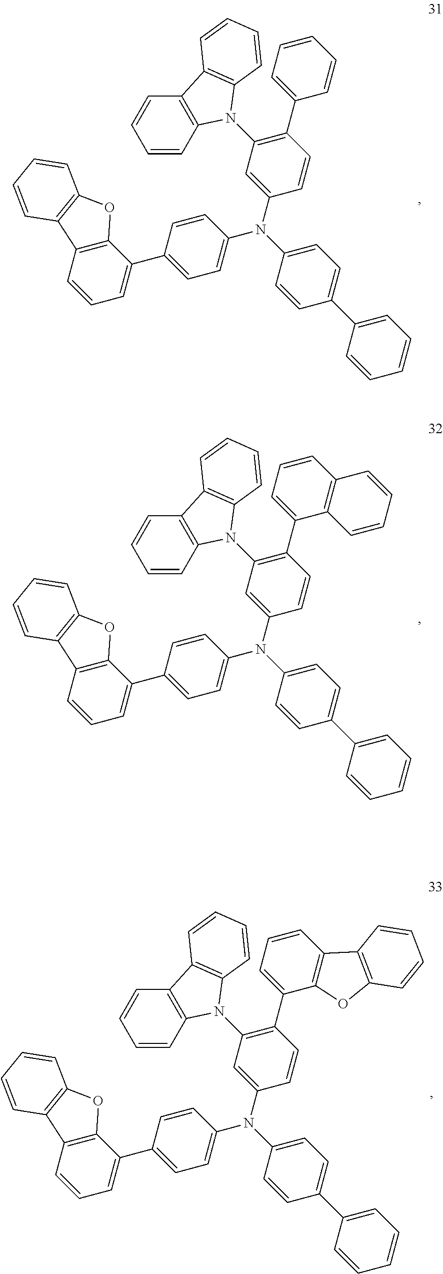 [SMILES: c1ccc(-c2ccc(N(c3ccc(-c4cccc5c4oc4ccccc45)cc3)c3ccc(-c4ccccc4)c(-n4c5ccccc5c5ccccc54)c3)cc2)cc1 ; c1ccc(-c2ccc(N(c3ccc(-c4cccc5c4oc4ccccc45)cc3)c3ccc(-c4cccc5ccccc45)c(-n4c5ccccc5c5ccccc54)c3)cc2)cc1 ; c1ccc(-c2ccc(N(c3ccc(-c4cccc5c4oc4ccccc45)cc3)c3ccc(-c4cccc5c4oc4ccccc45)c(-n4c5ccccc5c5ccccc54)c3)cc2)cc1]

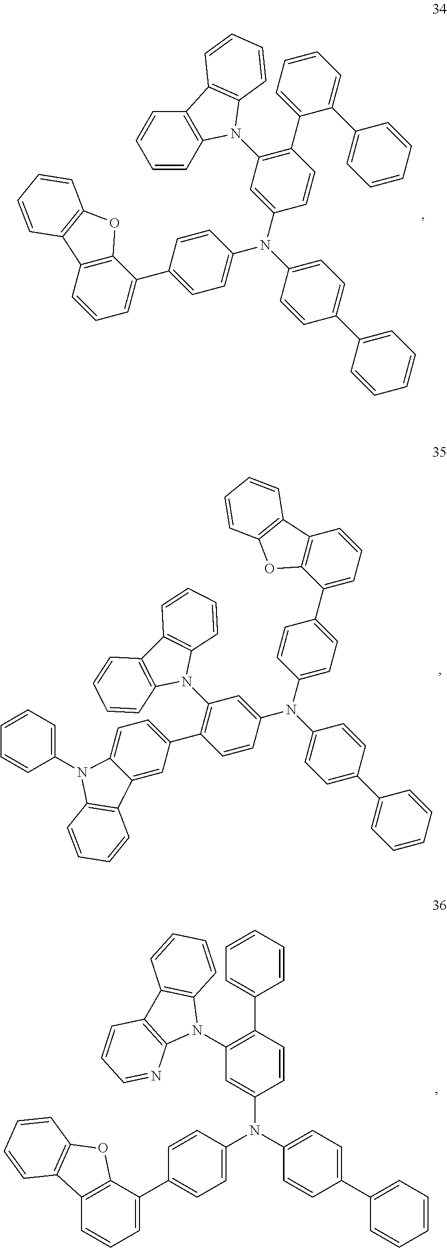 [SMILES: c1ccc(-c2ccc(N(c3ccc(-c4cccc5c4oc4ccccc45)cc3)c3ccc(-c4ccccc4-c4ccccc4)c(-n4c5ccccc5c5ccccc54)c3)cc2)cc1 ; c1ccc(-c2ccc(N(c3ccc(-c4cccc5c4oc4ccccc45)cc3)c3ccc(-c4ccc5c(c4)c4ccccc4n5-c4ccccc4)c(-n4c5ccccc5c5ccccc54)c3)cc2)cc1 ; c1ccc(-c2ccc(N(c3ccc(-c4cccc5c4oc4ccccc45)cc3)c3ccc(-c4ccccc4)c(-n4c5ccccc5c5cccnc54)c3)cc2)cc1]

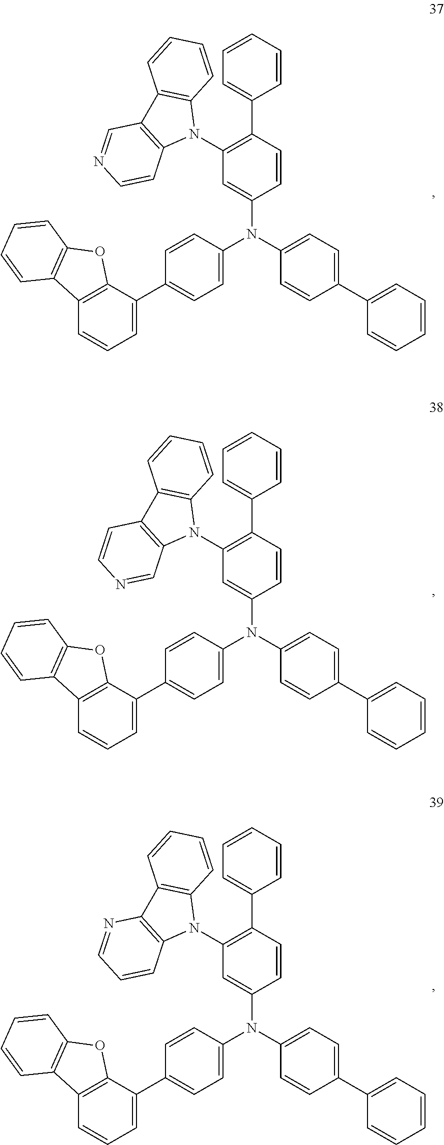 [SMILES: c1ccc(-c2ccc(N(c3ccc(-c4cccc5c4oc4ccccc45)cc3)c3ccc(-c4ccccc4)c(-n4c5ccccc5c5cnccc54)c3)cc2)cc1 ; c1ccc(-c2ccc(N(c3ccc(-c4cccc5c4oc4ccccc45)cc3)c3ccc(-c4ccccc4)c(-n4c5ccccc5c5ccncc54)c3)cc2)cc1 ; c1ccc(-c2ccc(N(c3ccc(-c4cccc5c4oc4ccccc45)cc3)c3ccc(-c4ccccc4)c(-n4c5ccccc5c5ncccc54)c3)cc2)cc1]

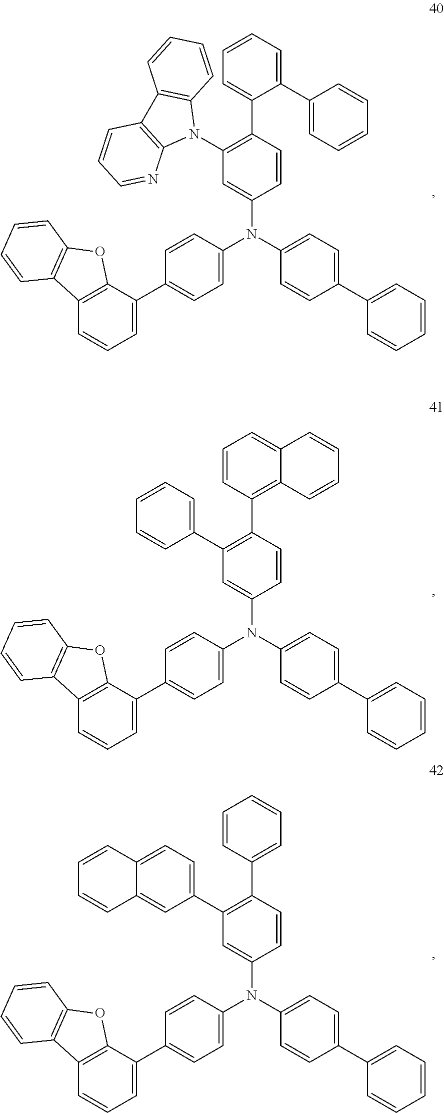 [SMILES: c1ccc(-c2ccc(N(c3ccc(-c4cccc5c4oc4ccccc45)cc3)c3ccc(-c4ccccc4-c4ccccc4)c(-n4c5ccccc5c5cccnc54)c3)cc2)cc1 ; c1ccc(-c2ccc(N(c3ccc(-c4cccc5c4oc4ccccc45)cc3)c3ccc(-c4cccc5ccccc45)c(-c4ccccc4)c3)cc2)cc1 ; c1ccc(-c2ccc(N(c3ccc(-c4cccc5c4oc4ccccc45)cc3)c3ccc(-c4ccccc4)c(-c4ccc5ccccc5c4)c3)cc2)cc1]

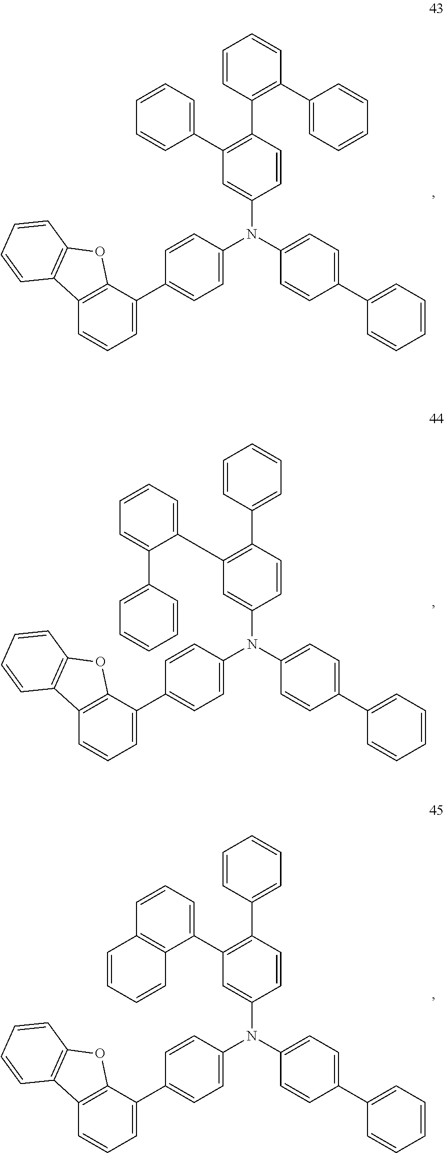 [SMILES: c1ccc(-c2ccc(N(c3ccc(-c4cccc5c4oc4ccccc45)cc3)c3ccc(-c4ccccc4-c4ccccc4)c(-c4ccccc4)c3)cc2)cc1 ; c1ccc(-c2ccc(N(c3ccc(-c4cccc5c4oc4ccccc45)cc3)c3ccc(-c4ccccc4)c(-c4ccccc4-c4ccccc4)c3)cc2)cc1 ; c1ccc(-c2ccc(N(c3ccc(-c4cccc5c4oc4ccccc45)cc3)c3ccc(-c4ccccc4)c(-c4cccc5ccccc45)c3)cc2)cc1]

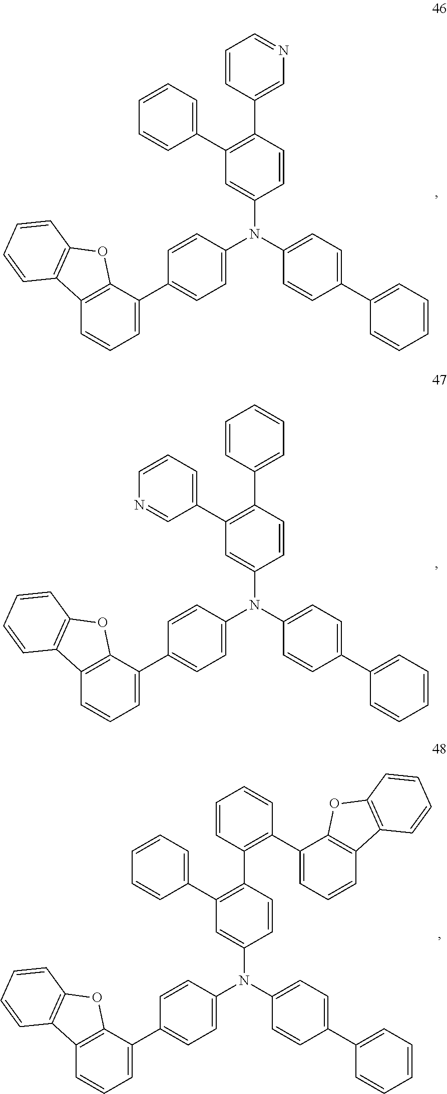 [SMILES: c1ccc(-c2ccc(N(c3ccc(-c4cccc5c4oc4ccccc45)cc3)c3ccc(-c4cccnc4)c(-c4ccccc4)c3)cc2)cc1 ; c1ccc(-c2ccc(N(c3ccc(-c4cccc5c4oc4ccccc45)cc3)c3ccc(-c4ccccc4)c(-c4cccnc4)c3)cc2)cc1 ; c1ccc(-c2ccc(N(c3ccc(-c4cccc5c4oc4ccccc45)cc3)c3ccc(-c4ccccc4-c4cccc5c4oc4ccccc45)c(-c4ccccc4)c3)cc2)cc1]

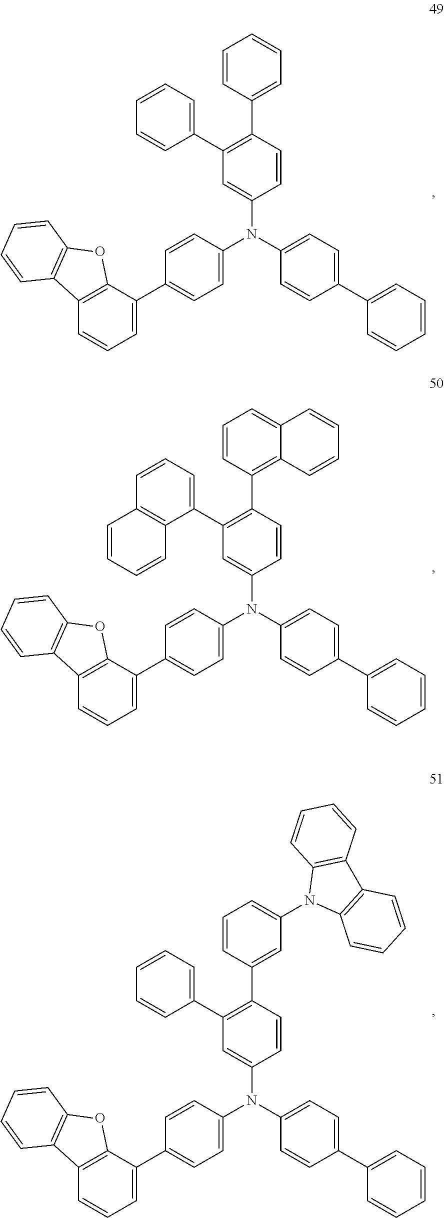 [SMILES: c1ccc(-c2ccc(N(c3ccc(-c4cccc5c4oc4ccccc45)cc3)c3ccc(-c4ccccc4)c(-c4ccccc4)c3)cc2)cc1 ; c1ccc(-c2ccc(N(c3ccc(-c4cccc5c4oc4ccccc45)cc3)c3ccc(-c4cccc5ccccc45)c(-c4cccc5ccccc45)c3)cc2)cc1 ; c1ccc(-c2ccc(N(c3ccc(-c4cccc5c4oc4ccccc45)cc3)c3ccc(-c4cccc(-n5c6ccccc6c6ccccc65)c4)c(-c4ccccc4)c3)cc2)cc1]

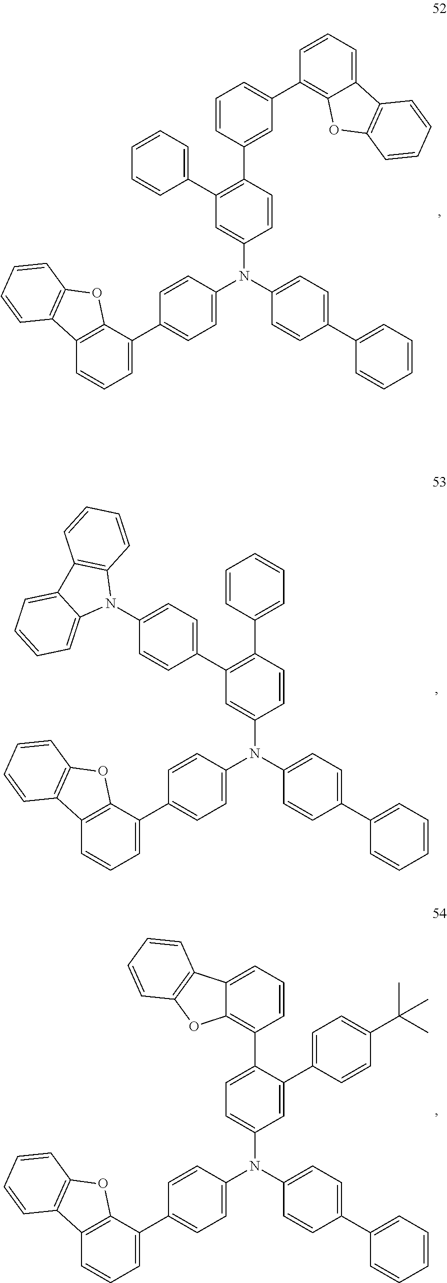 [SMILES: c1ccc(-c2ccc(N(c3ccc(-c4cccc5c4oc4ccccc45)cc3)c3ccc(-c4cccc(-c5cccc6c5oc5ccccc56)c4)c(-c4ccccc4)c3)cc2)cc1 ; c1ccc(-c2ccc(N(c3ccc(-c4cccc5c4oc4ccccc45)cc3)c3ccc(-c4ccccc4)c(-c4ccc(-n5c6ccccc6c6ccccc65)cc4)c3)cc2)cc1 ; CC(C)(C)c1ccc(-c2cc(N(c3ccc(-c4ccccc4)cc3)c3ccc(-c4cccc5c4oc4ccccc45)cc3)ccc2-c2cccc3c2oc2ccccc23)cc1]

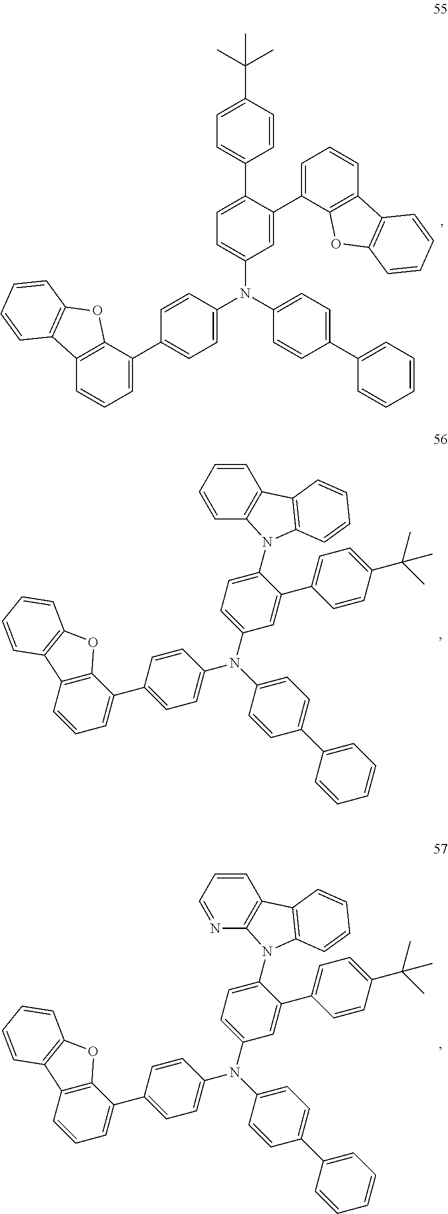 [SMILES: CC(C)(C)c1ccc(-c2ccc(N(c3ccc(-c4ccccc4)cc3)c3ccc(-c4cccc5c4oc4ccccc45)cc3)cc2-c2cccc3c2oc2ccccc23)cc1 ; CC(C)(C)c1ccc(-c2cc(N(c3ccc(-c4ccccc4)cc3)c3ccc(-c4cccc5c4oc4ccccc45)cc3)ccc2-n2c3ccccc3c3ccccc32)cc1 ; CC(C)(C)c1ccc(-c2cc(N(c3ccc(-c4ccccc4)cc3)c3ccc(-c4cccc5c4oc4ccccc45)cc3)ccc2-n2c3ccccc3c3cccnc32)cc1]

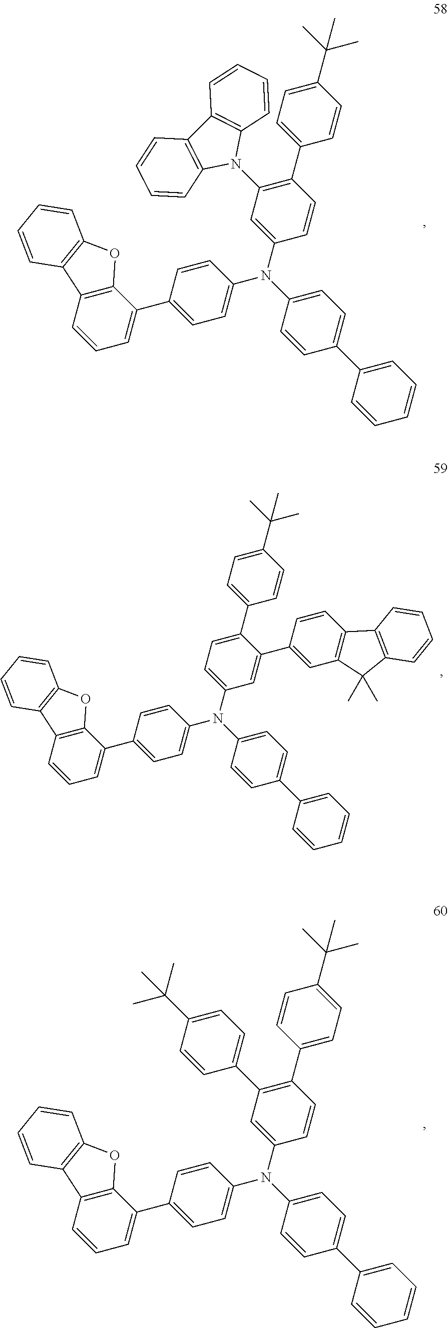 [SMILES: CC(C)(C)c1ccc(-c2ccc(N(c3ccc(-c4ccccc4)cc3)c3ccc(-c4cccc5c4oc4ccccc45)cc3)cc2-n2c3ccccc3c3ccccc32)cc1 ; CC(C)(C)c1ccc(-c2ccc(N(c3ccc(-c4ccccc4)cc3)c3ccc(-c4cccc5c4oc4ccccc45)cc3)cc2-c2ccc3c(c2)C(C)(C)c2ccccc2-3)cc1 ; CC(C)(C)c1ccc(-c2ccc(N(c3ccc(-c4ccccc4)cc3)c3ccc(-c4cccc5c4oc4ccccc45)cc3)cc2-c2ccc(C(C)(C)C)cc2)cc1]

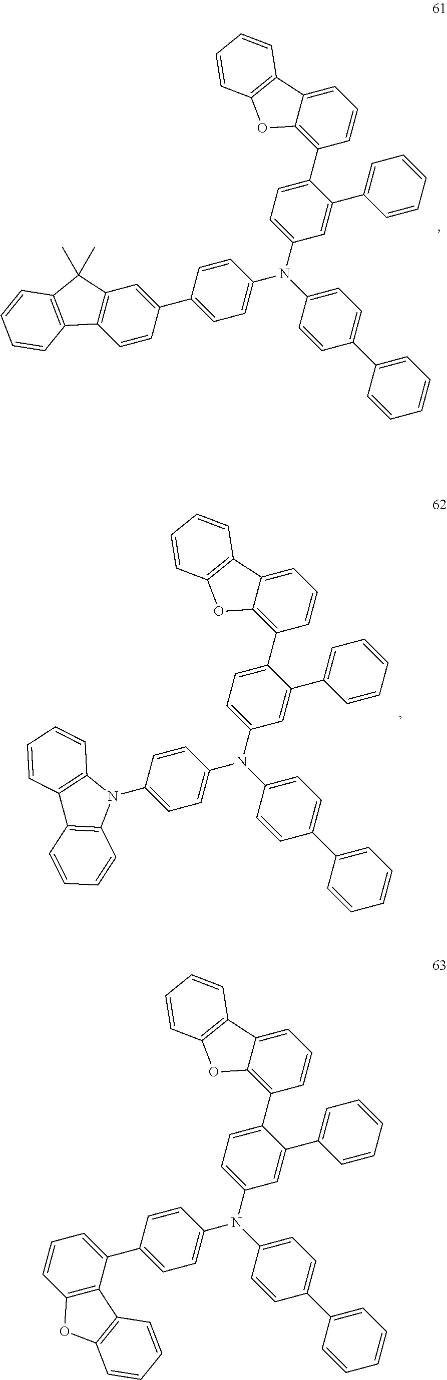 [SMILES: CC1(C)c2ccccc2-c2ccc(-c3ccc(N(c4ccc(-c5ccccc5)cc4)c4ccc(-c5cccc6c5oc5ccccc56)c(-c5ccccc5)c4)cc3)cc21 ; c1ccc(-c2ccc(N(c3ccc(-n4c5ccccc5c5ccccc54)cc3)c3ccc(-c4cccc5c4oc4ccccc45)c(-c4ccccc4)c3)cc2)cc1 ; c1ccc(-c2ccc(N(c3ccc(-c4cccc5oc6ccccc6c45)cc3)c3ccc(-c4cccc5c4oc4ccccc45)c(-c4ccccc4)c3)cc2)cc1]

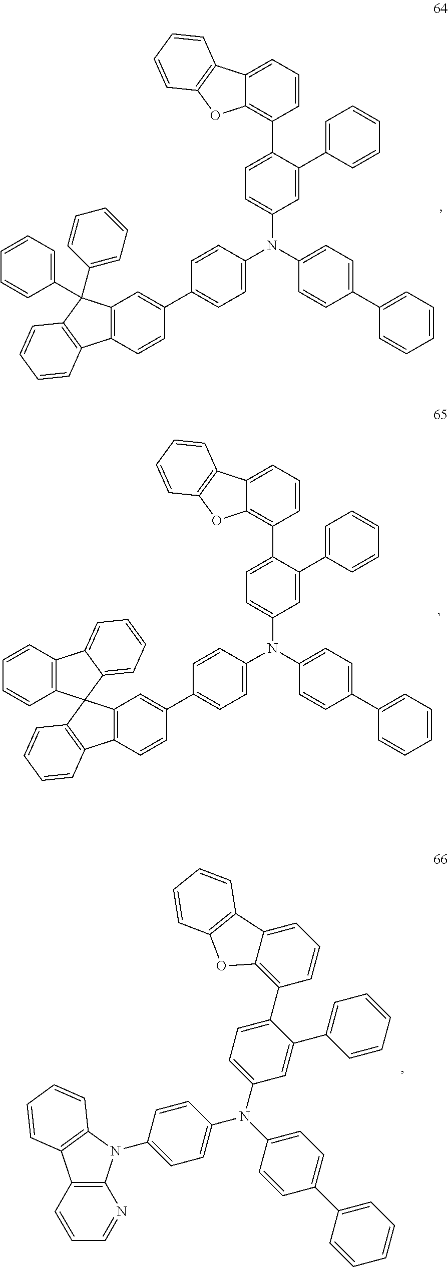 [SMILES: c1ccc(-c2ccc(N(c3ccc(-c4ccc5c(c4)C(c4ccccc4)(c4ccccc4)c4ccccc4-5)cc3)c3ccc(-c4cccc5c4oc4ccccc45)c(-c4ccccc4)c3)cc2)cc1 ; c1ccc(-c2ccc(N(c3ccc(-c4ccc5c(c4)C4(c6ccccc6-c6ccccc64)c4ccccc4-5)cc3)c3ccc(-c4cccc5c4oc4ccccc45)c(-c4ccccc4)c3)cc2)cc1 ; c1ccc(-c2ccc(N(c3ccc(-n4c5ccccc5c5cccnc54)cc3)c3ccc(-c4cccc5c4oc4ccccc45)c(-c4ccccc4)c3)cc2)cc1]

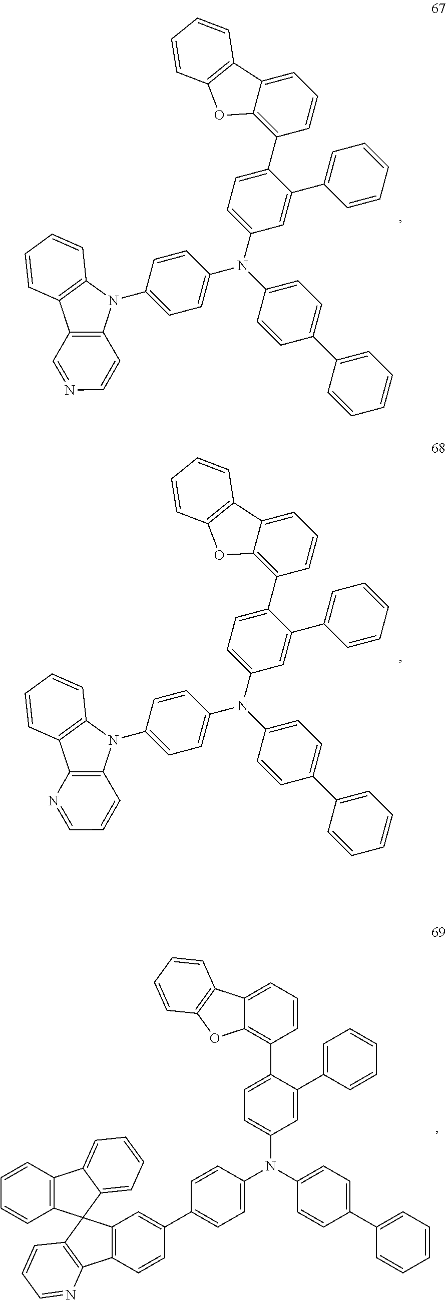 [SMILES: c1ccc(-c2ccc(N(c3ccc(-n4c5ccccc5c5cnccc54)cc3)c3ccc(-c4cccc5c4oc4ccccc45)c(-c4ccccc4)c3)cc2)cc1 ; c1ccc(-c2ccc(N(c3ccc(-n4c5ccccc5c5ncccc54)cc3)c3ccc(-c4cccc5c4oc4ccccc45)c(-c4ccccc4)c3)cc2)cc1 ; c1ccc(-c2ccc(N(c3ccc(-c4ccc5c(c4)C4(c6ccccc6-c6ccccc64)c4cccnc4-5)cc3)c3ccc(-c4cccc5c4oc4ccccc45)c(-c4ccccc4)c3)cc2)cc1]

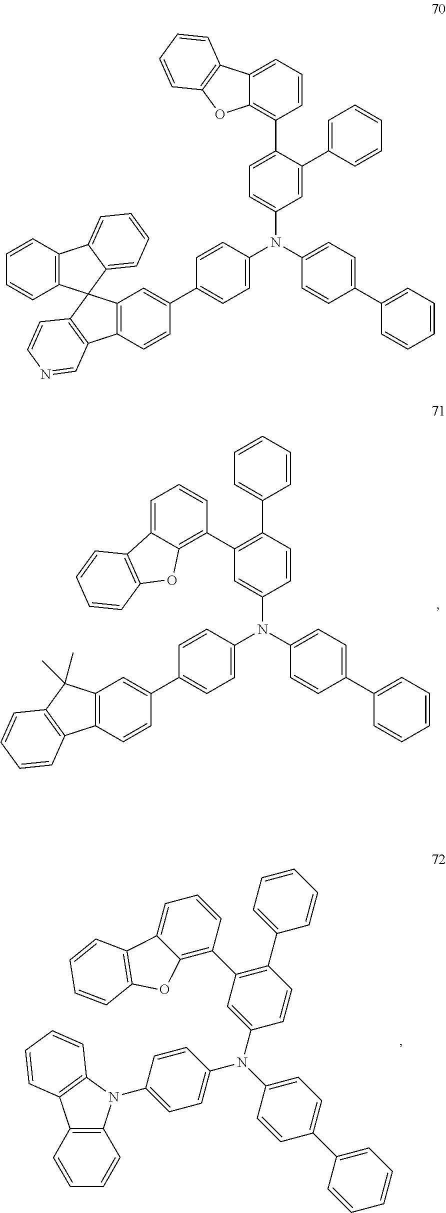 [SMILES: c1ccc(-c2ccc(N(c3ccc(-c4ccc5c(c4)C4(c6ccccc6-c6ccccc64)c4ccncc4-5)cc3)c3ccc(-c4cccc5c4oc4ccccc45)c(-c4ccccc4)c3)cc2)cc1 ; CC1(C)c2ccccc2-c2ccc(-c3ccc(N(c4ccc(-c5ccccc5)cc4)c4ccc(-c5ccccc5)c(-c5cccc6c5oc5ccccc56)c4)cc3)cc21 ; c1ccc(-c2ccc(N(c3ccc(-n4c5ccccc5c5ccccc54)cc3)c3ccc(-c4ccccc4)c(-c4cccc5c4oc4ccccc45)c3)cc2)cc1]

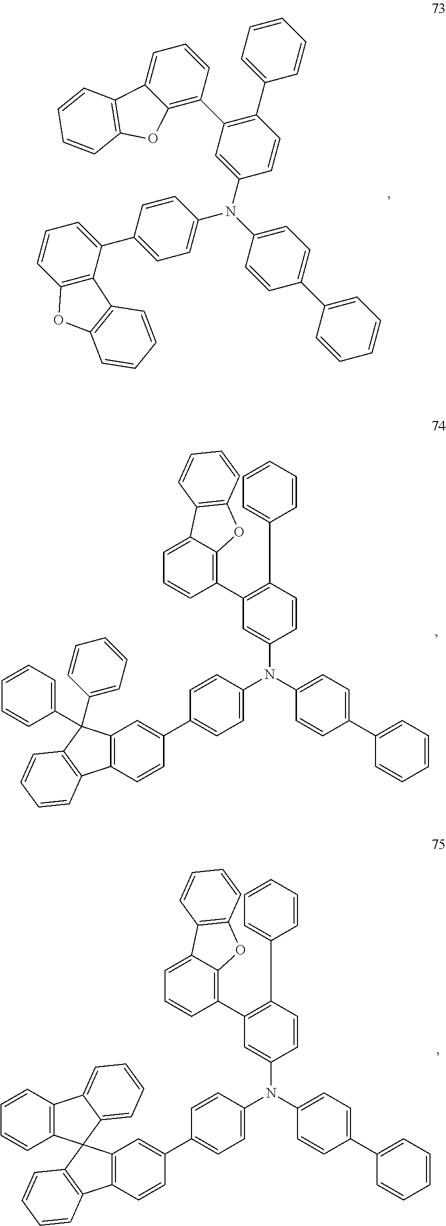 [SMILES: c1ccc(-c2ccc(N(c3ccc(-c4cccc5oc6ccccc6c45)cc3)c3ccc(-c4ccccc4)c(-c4cccc5c4oc4ccccc45)c3)cc2)cc1 ; c1ccc(-c2ccc(N(c3ccc(-c4ccc5c(c4)C(c4ccccc4)(c4ccccc4)c4ccccc4-5)cc3)c3ccc(-c4ccccc4)c(-c4cccc5c4oc4ccccc45)c3)cc2)cc1 ; c1ccc(-c2ccc(N(c3ccc(-c4ccc5c(c4)C4(c6ccccc6-c6ccccc64)c4ccccc4-5)cc3)c3ccc(-c4ccccc4)c(-c4cccc5c4oc4ccccc45)c3)cc2)cc1]

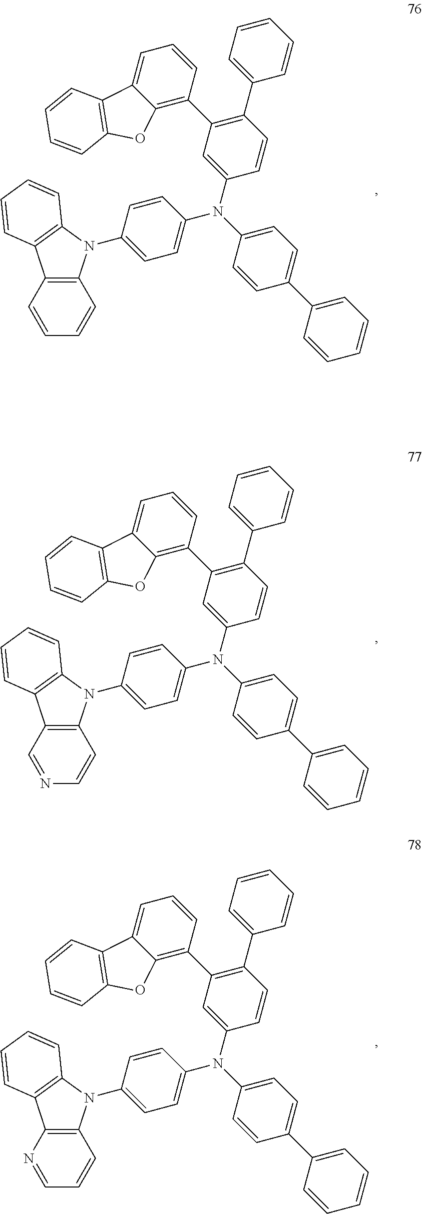 [SMILES: c1ccc(-c2ccc(N(c3ccc(-n4c5ccccc5c5ccccc54)cc3)c3ccc(-c4ccccc4)c(-c4cccc5c4oc4ccccc45)c3)cc2)cc1 ; c1ccc(-c2ccc(N(c3ccc(-n4c5ccccc5c5cnccc54)cc3)c3ccc(-c4ccccc4)c(-c4cccc5c4oc4ccccc45)c3)cc2)cc1 ; c1ccc(-c2ccc(N(c3ccc(-n4c5ccccc5c5ncccc54)cc3)c3ccc(-c4ccccc4)c(-c4cccc5c4oc4ccccc45)c3)cc2)cc1]

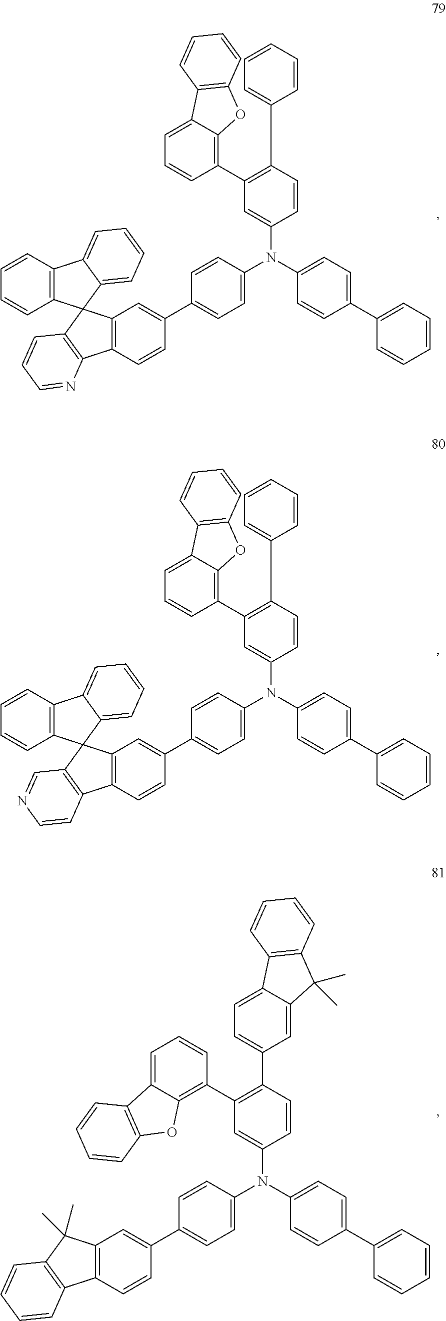 [SMILES: c1ccc(-c2ccc(N(c3ccc(-c4ccc5c(c4)C4(c6ccccc6-c6ccccc64)c4cccnc4-5)cc3)c3ccc(-c4ccccc4)c(-c4cccc5c4oc4ccccc45)c3)cc2)cc1 ; c1ccc(-c2ccc(N(c3ccc(-c4ccc5c(c4)C4(c6ccccc6-c6ccccc64)c4cnccc4-5)cc3)c3ccc(-c4ccccc4)c(-c4cccc5c4oc4ccccc45)c3)cc2)cc1 ; CC1(C)c2ccccc2-c2ccc(-c3ccc(N(c4ccc(-c5ccccc5)cc4)c4ccc(-c5ccc6c(c5)C(C)(C)c5ccccc5-6)c(-c5cccc6c5oc5ccccc56)c4)cc3)cc21]

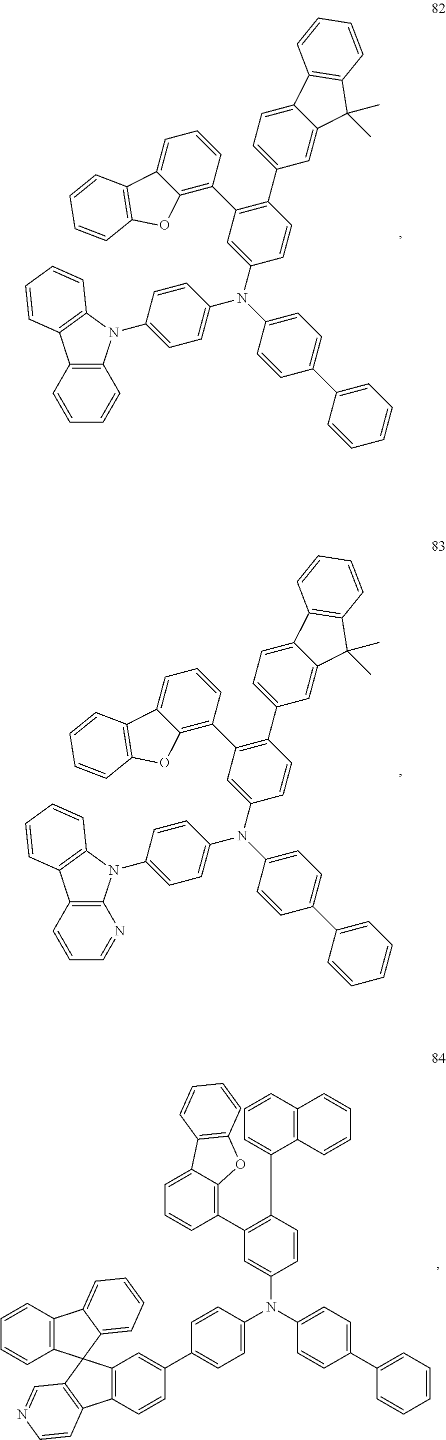 [SMILES: CC1(C)c2ccccc2-c2ccc(-c3ccc(N(c4ccc(-c5ccccc5)cc4)c4ccc(-n5c6ccccc6c6ccccc65)cc4)cc3-c3cccc4c3oc3ccccc34)cc21 ; CC1(C)c2ccccc2-c2ccc(-c3ccc(N(c4ccc(-c5ccccc5)cc4)c4ccc(-n5c6ccccc6c6cccnc65)cc4)cc3-c3cccc4c3oc3ccccc34)cc21 ; c1ccc(-c2ccc(N(c3ccc(-c4ccc5c(c4)C4(c6ccccc6-c6ccccc64)c4cnccc4-5)cc3)c3ccc(-c4cccc5ccccc45)c(-c4cccc5c4oc4ccccc45)c3)cc2)cc1]

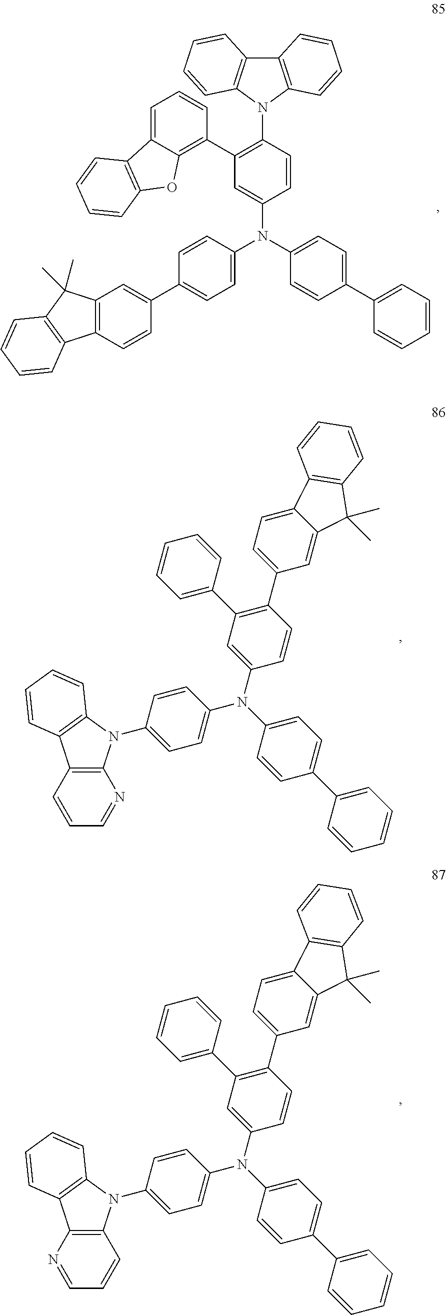

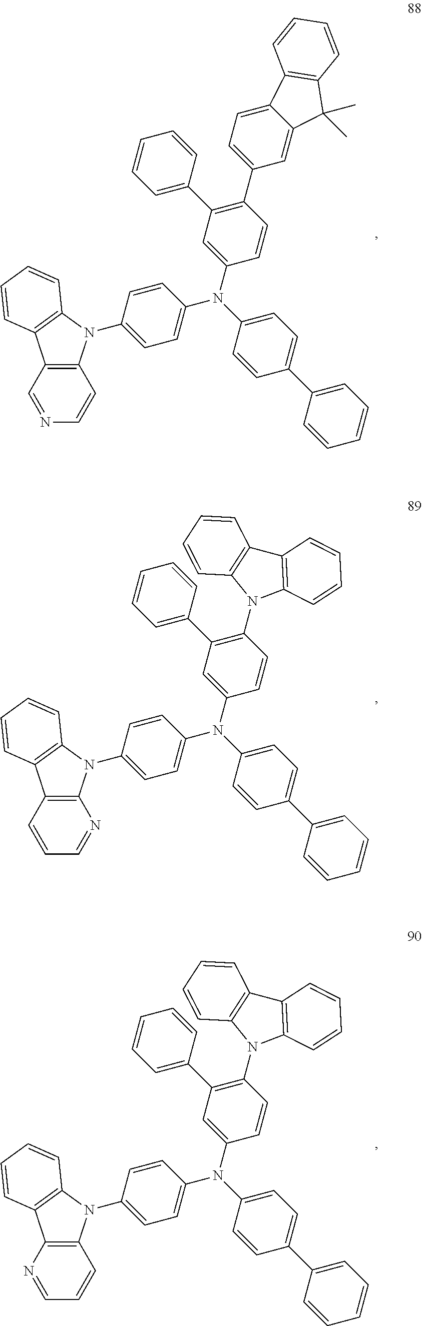 [SMILES: CC1(C)c2ccccc2-c2ccc(-c3ccc(N(c4ccc(-c5ccccc5)cc4)c4ccc(-n5c6ccccc6c6cnccc65)cc4)cc3-c3ccccc3)cc21 ; c1ccc(-c2ccc(N(c3ccc(-n4c5ccccc5c5cccnc54)cc3)c3ccc(-n4c5ccccc5c5ccccc54)c(-c4ccccc4)c3)cc2)cc1 ; c1ccc(-c2ccc(N(c3ccc(-n4c5ccccc5c5ncccc54)cc3)c3ccc(-n4c5ccccc5c5ccccc54)c(-c4ccccc4)c3)cc2)cc1]

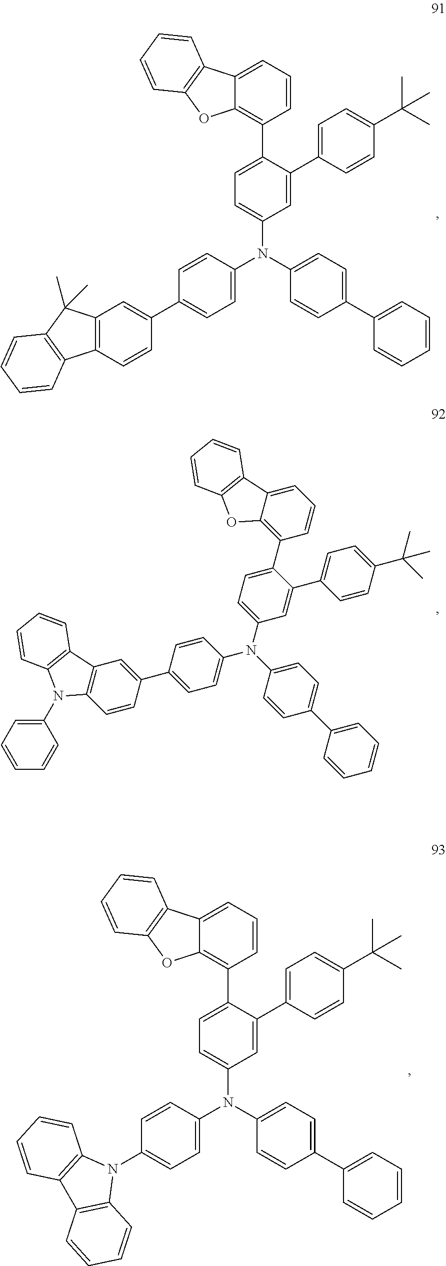 [SMILES: CC(C)(C)c1ccc(-c2cc(N(c3ccc(-c4ccccc4)cc3)c3ccc(-c4ccc5c(c4)C(C)(C)c4ccccc4-5)cc3)ccc2-c2cccc3c2oc2ccccc23)cc1 ; CC(C)(C)c1ccc(-c2cc(N(c3ccc(-c4ccccc4)cc3)c3ccc(-c4ccc5c(c4)c4ccccc4n5-c4ccccc4)cc3)ccc2-c2cccc3c2oc2ccccc23)cc1 ; CC(C)(C)c1ccc(-c2cc(N(c3ccc(-c4ccccc4)cc3)c3ccc(-n4c5ccccc5c5ccccc54)cc3)ccc2-c2cccc3c2oc2ccccc23)cc1]

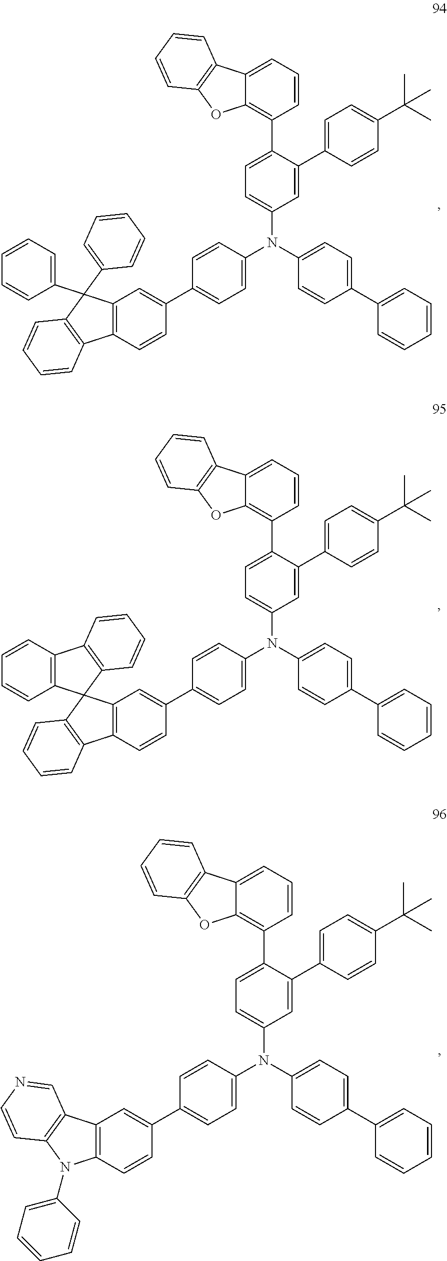 [SMILES: CC(C)(C)c1ccc(-c2cc(N(c3ccc(-c4ccccc4)cc3)c3ccc(-c4ccc5c(c4)C(c4ccccc4)(c4ccccc4)c4ccccc4-5)cc3)ccc2-c2cccc3c2oc2ccccc23)cc1 ; CC(C)(C)c1ccc(-c2cc(N(c3ccc(-c4ccccc4)cc3)c3ccc(-c4ccc5c(c4)C4(c6ccccc6-c6ccccc64)c4ccccc4-5)cc3)ccc2-c2cccc3c2oc2ccccc23)cc1 ; CC(C)(C)c1ccc(-c2cc(N(c3ccc(-c4ccccc4)cc3)c3ccc(-c4ccc5c(c4)c4cnccc4n5-c4ccccc4)cc3)ccc2-c2cccc3c2oc2ccccc23)cc1]

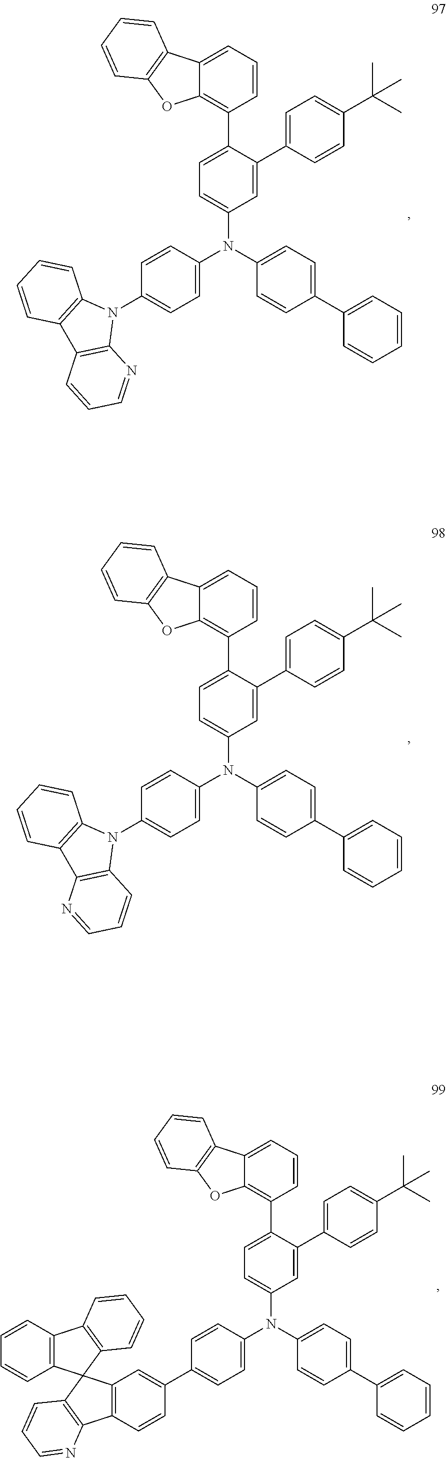 [SMILES: CC(C)(C)c1ccc(-c2cc(N(c3ccc(-c4ccccc4)cc3)c3ccc(-n4c5ccccc5c5cccnc54)cc3)ccc2-c2cccc3c2oc2ccccc23)cc1 ; CC(C)(C)c1ccc(-c2cc(N(c3ccc(-c4ccccc4)cc3)c3ccc(-n4c5ccccc5c5ncccc54)cc3)ccc2-c2cccc3c2oc2ccccc23)cc1 ; CC(C)(C)c1ccc(-c2cc(N(c3ccc(-c4ccccc4)cc3)c3ccc(-c4ccc5c(c4)C4(c6ccccc6-c6ccccc64)c4cccnc4-5)cc3)ccc2-c2cccc3c2oc2ccccc23)cc1]

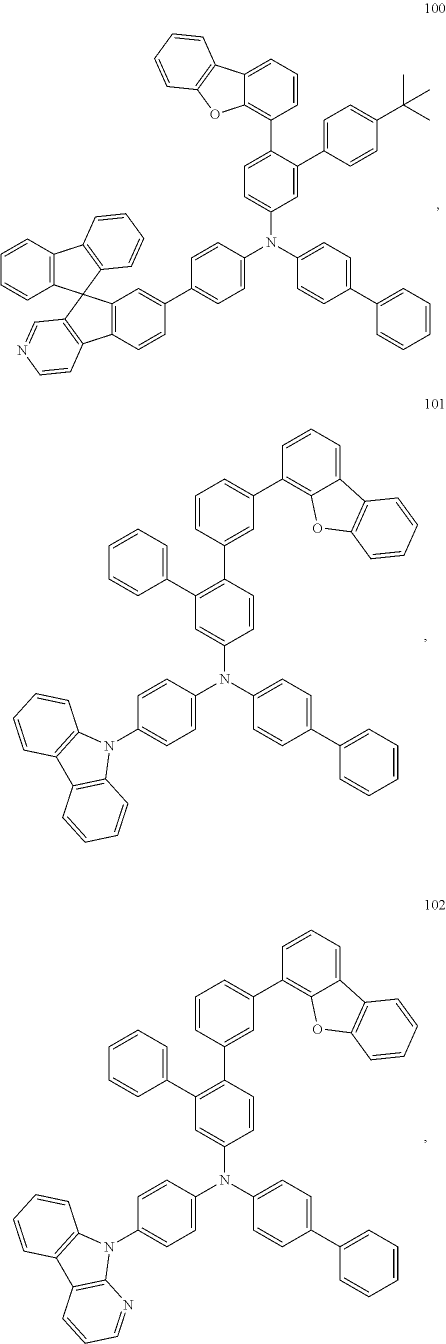 [SMILES: CC(C)(C)c1ccc(-c2cc(N(c3ccc(-c4ccccc4)cc3)c3ccc(-c4ccc5c(c4)C4(c6ccccc6-c6ccccc64)c4cnccc4-5)cc3)ccc2-c2cccc3c2oc2ccccc23)cc1 ; c1ccc(-c2ccc(N(c3ccc(-n4c5ccccc5c5ccccc54)cc3)c3ccc(-c4cccc(-c5cccc6c5oc5ccccc56)c4)c(-c4ccccc4)c3)cc2)cc1 ; c1ccc(-c2ccc(N(c3ccc(-n4c5ccccc5c5cccnc54)cc3)c3ccc(-c4cccc(-c5cccc6c5oc5ccccc56)c4)c(-c4ccccc4)c3)cc2)cc1]

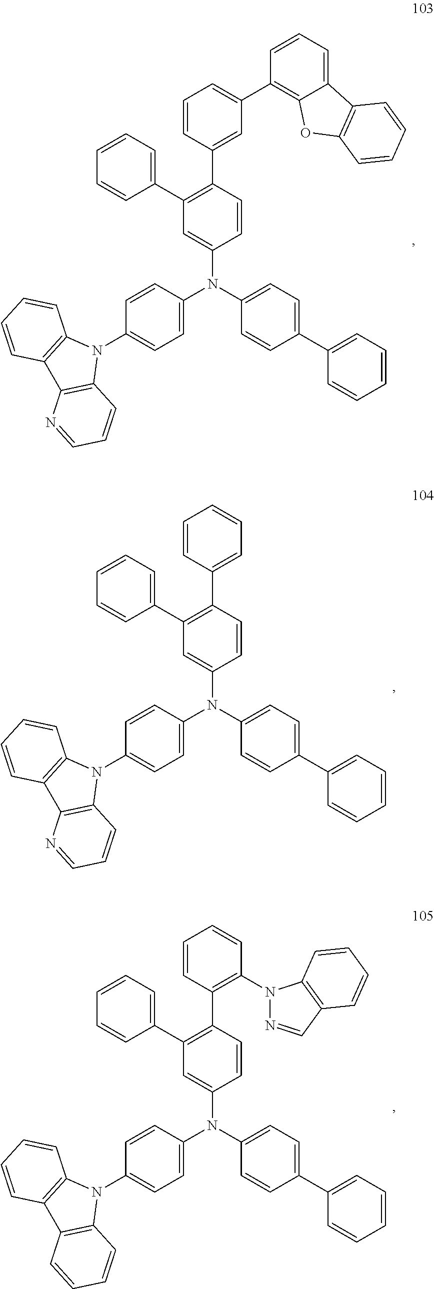 [SMILES: c1ccc(-c2ccc(N(c3ccc(-n4c5ccccc5c5ncccc54)cc3)c3ccc(-c4cccc(-c5cccc6c5oc5ccccc56)c4)c(-c4ccccc4)c3)cc2)cc1 ; c1ccc(-c2ccc(N(c3ccc(-n4c5ccccc5c5ncccc54)cc3)c3ccc(-c4ccccc4)c(-c4ccccc4)c3)cc2)cc1 ; c1ccc(-c2ccc(N(c3ccc(-n4c5ccccc5c5ccccc54)cc3)c3ccc(-c4ccccc4-n4ncc5ccccc54)c(-c4ccccc4)c3)cc2)cc1]

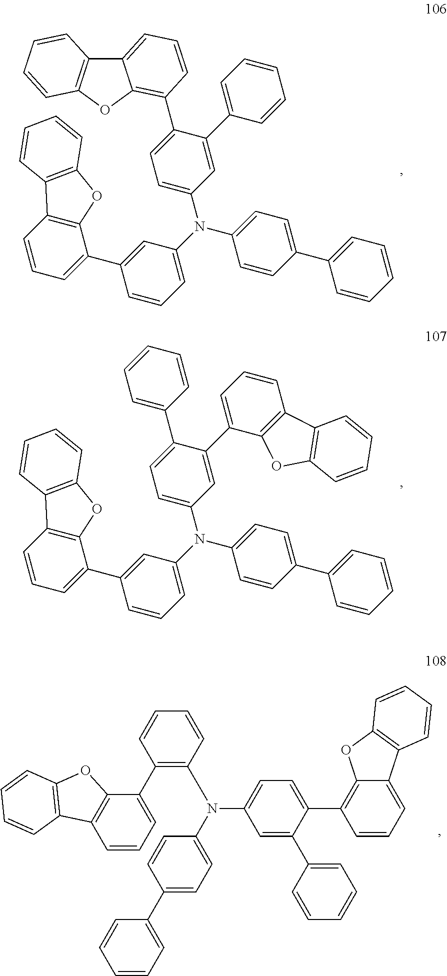

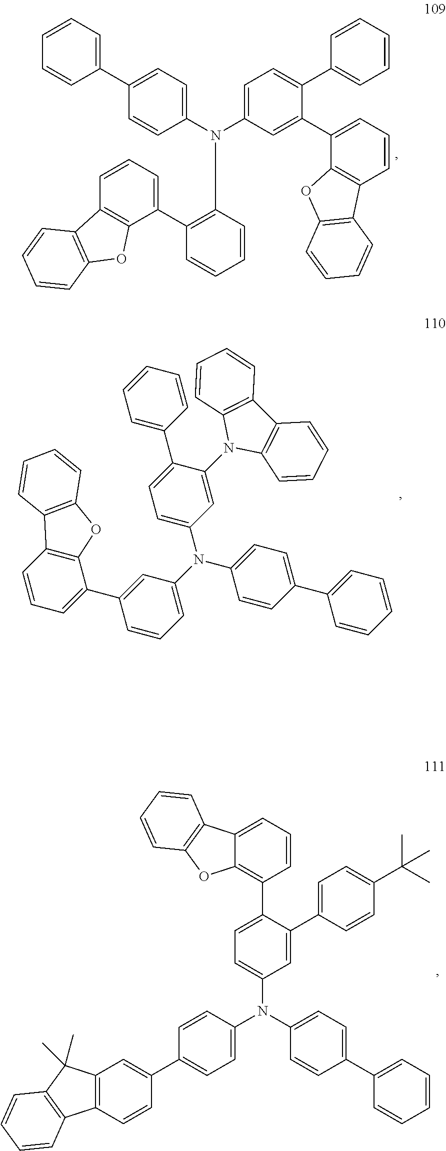 [SMILES: c1ccc(-c2ccc(N(c3ccc(-c4ccccc4)c(-c4cccc5c4oc4ccccc45)c3)c3ccccc3-c3cccc4c3oc3ccccc34)cc2)cc1 ; c1ccc(-c2ccc(N(c3cccc(-c4cccc5c4oc4ccccc45)c3)c3ccc(-c4ccccc4)c(-n4c5ccccc5c5ccccc54)c3)cc2)cc1 ; CC(C)(C)c1ccc(-c2cc(N(c3ccc(-c4ccccc4)cc3)c3ccc(-c4ccc5c(c4)C(C)(C)c4ccccc4-5)cc3)ccc2-c2cccc3c2oc2ccccc23)cc1]

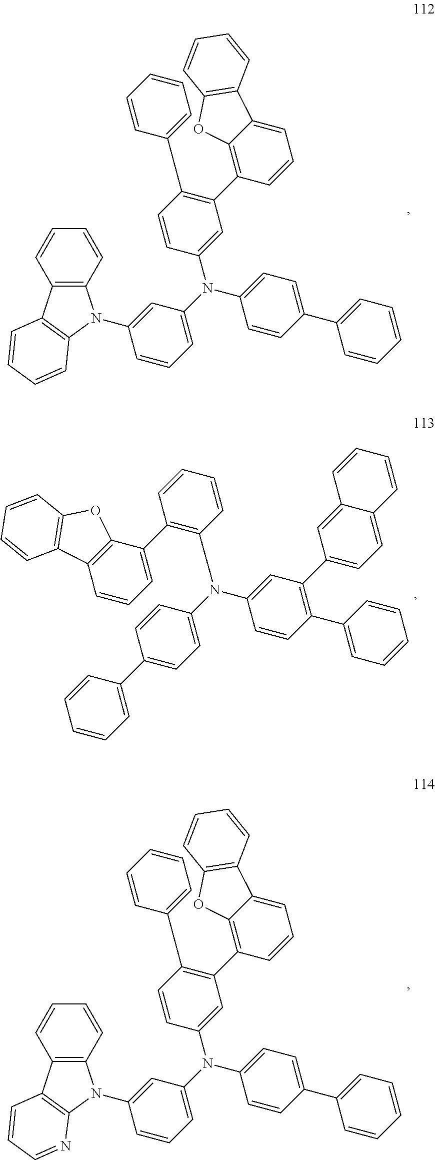 [SMILES: c1ccc(-c2ccc(N(c3cccc(-n4c5ccccc5c5ccccc54)c3)c3ccc(-c4ccccc4)c(-c4cccc5c4oc4ccccc45)c3)cc2)cc1 ; c1ccc(-c2ccc(N(c3ccc(-c4ccccc4)c(-c4ccc5ccccc5c4)c3)c3ccccc3-c3cccc4c3oc3ccccc34)cc2)cc1 ; c1ccc(-c2ccc(N(c3cccc(-n4c5ccccc5c5cccnc54)c3)c3ccc(-c4ccccc4)c(-c4cccc5c4oc4ccccc45)c3)cc2)cc1]

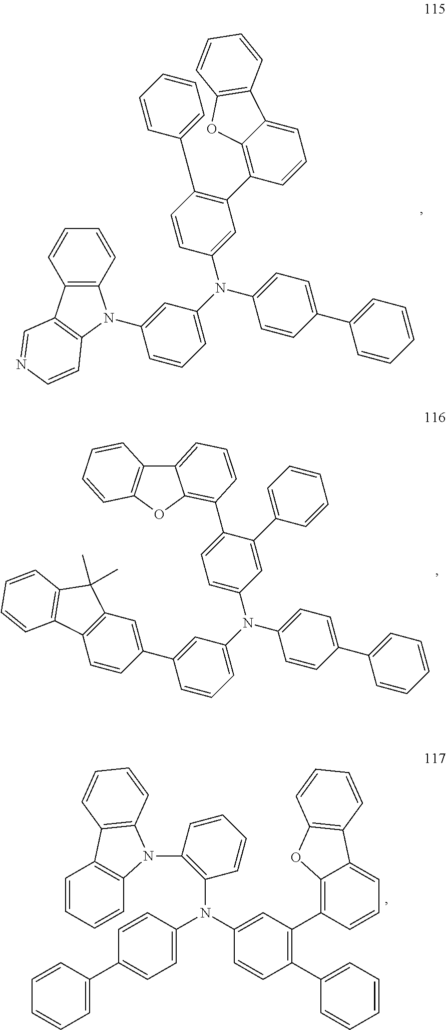 [SMILES: c1ccc(-c2ccc(N(c3cccc(-n4c5ccccc5c5cnccc54)c3)c3ccc(-c4ccccc4)c(-c4cccc5c4oc4ccccc45)c3)cc2)cc1 ; CC1(C)c2ccccc2-c2ccc(-c3cccc(N(c4ccc(-c5ccccc5)cc4)c4ccc(-c5cccc6c5oc5ccccc56)c(-c5ccccc5)c4)c3)cc21 ; c1ccc(-c2ccc(N(c3ccc(-c4ccccc4)c(-c4cccc5c4oc4ccccc45)c3)c3ccccc3-n3c4ccccc4c4ccccc43)cc2)cc1]

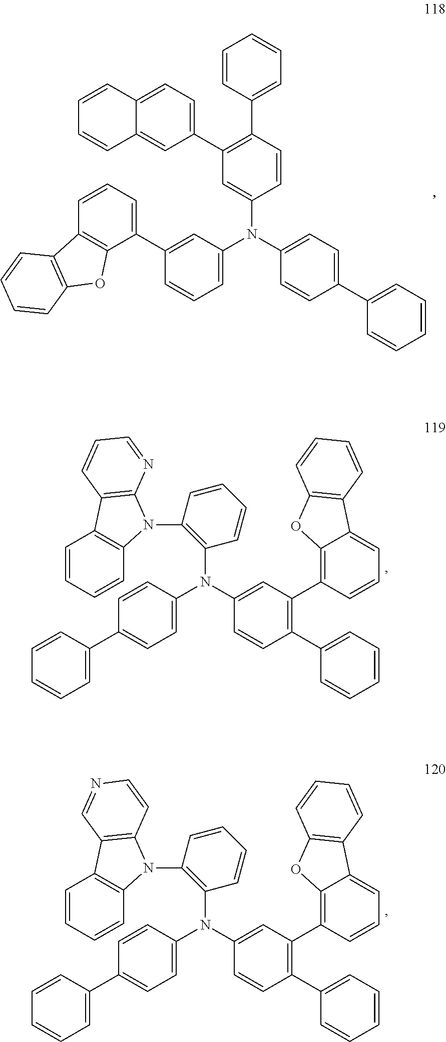 [SMILES: c1ccc(-c2ccc(N(c3cccc(-c4cccc5c4oc4ccccc45)c3)c3ccc(-c4ccccc4)c(-c4ccc5ccccc5c4)c3)cc2)cc1 ; c1ccc(-c2ccc(N(c3ccc(-c4ccccc4)c(-c4cccc5c4oc4ccccc45)c3)c3ccccc3-n3c4ccccc4c4cccnc43)cc2)cc1 ; c1ccc(-c2ccc(N(c3ccc(-c4ccccc4)c(-c4cccc5c4oc4ccccc45)c3)c3ccccc3-n3c4ccccc4c4cnccc43)cc2)cc1]

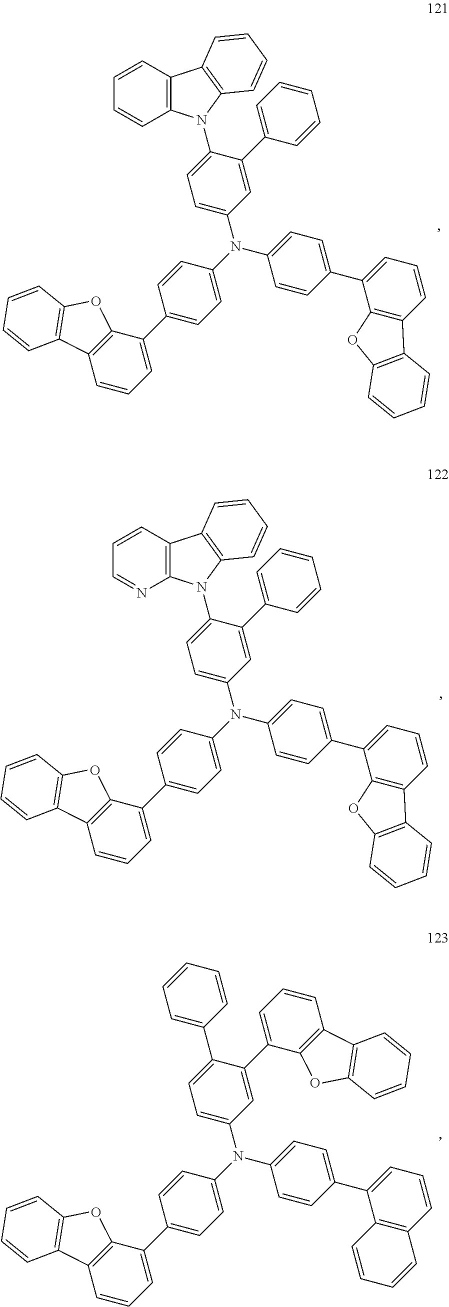 [SMILES: c1ccc(-c2cc(N(c3ccc(-c4cccc5c4oc4ccccc45)cc3)c3ccc(-c4cccc5c4oc4ccccc45)cc3)ccc2-n2c3ccccc3c3ccccc32)cc1 ; c1ccc(-c2cc(N(c3ccc(-c4cccc5c4oc4ccccc45)cc3)c3ccc(-c4cccc5c4oc4ccccc45)cc3)ccc2-n2c3ccccc3c3cccnc32)cc1 ; c1ccc(-c2ccc(N(c3ccc(-c4cccc5ccccc45)cc3)c3ccc(-c4cccc5c4oc4ccccc45)cc3)cc2-c2cccc3c2oc2ccccc23)cc1]

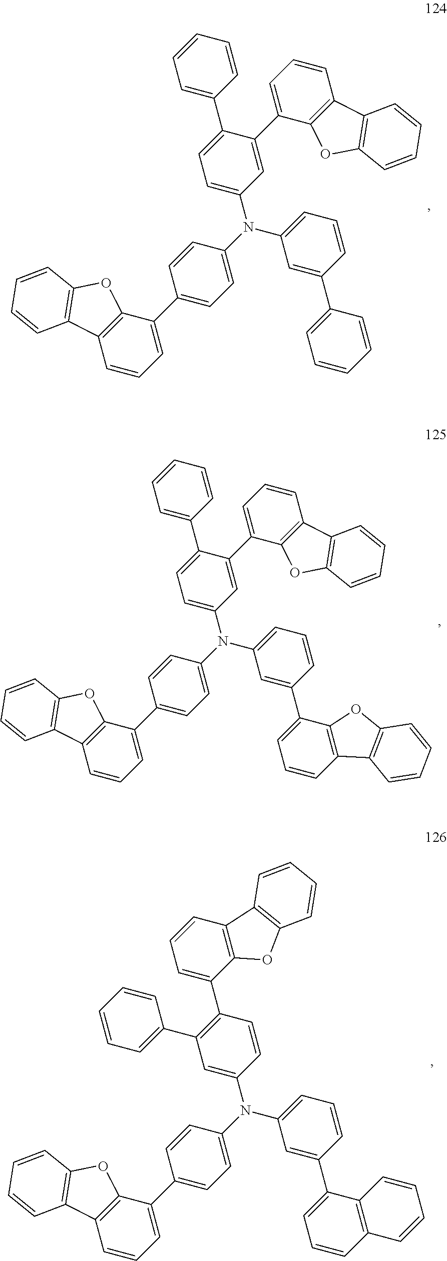 [SMILES: c1ccc(-c2cccc(N(c3ccc(-c4cccc5c4oc4ccccc45)cc3)c3ccc(-c4ccccc4)c(-c4cccc5c4oc4ccccc45)c3)c2)cc1 ; c1ccc(-c2ccc(N(c3ccc(-c4cccc5c4oc4ccccc45)cc3)c3cccc(-c4cccc5c4oc4ccccc45)c3)cc2-c2cccc3c2oc2ccccc23)cc1 ; c1ccc(-c2cc(N(c3ccc(-c4cccc5c4oc4ccccc45)cc3)c3cccc(-c4cccc5ccccc45)c3)ccc2-c2cccc3c2oc2ccccc23)cc1]

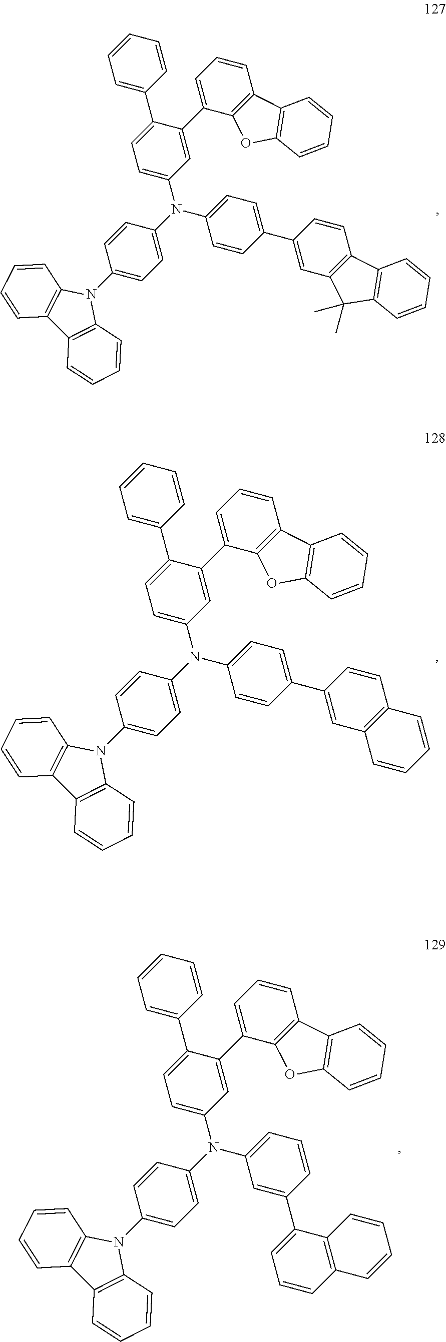 [SMILES: CC1(C)c2ccccc2-c2ccc(-c3ccc(N(c4ccc(-n5c6ccccc6c6ccccc65)cc4)c4ccc(-c5ccccc5)c(-c5cccc6c5oc5ccccc56)c4)cc3)cc21 ; c1ccc(-c2ccc(N(c3ccc(-c4ccc5ccccc5c4)cc3)c3ccc(-n4c5ccccc5c5ccccc54)cc3)cc2-c2cccc3c2oc2ccccc23)cc1 ; c1ccc(-c2ccc(N(c3ccc(-n4c5ccccc5c5ccccc54)cc3)c3cccc(-c4cccc5ccccc45)c3)cc2-c2cccc3c2oc2ccccc23)cc1]

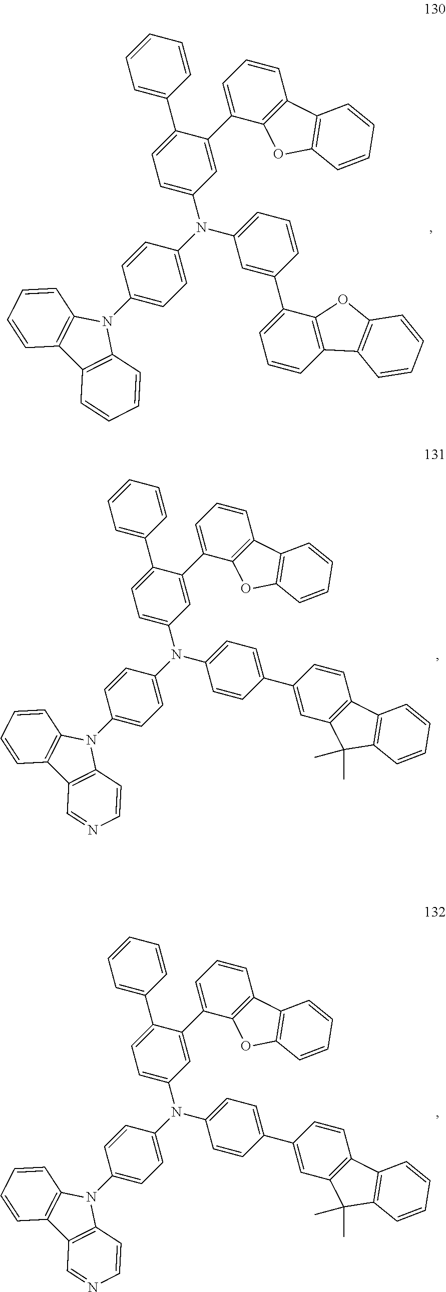 [SMILES: c1ccc(-c2ccc(N(c3ccc(-n4c5ccccc5c5ccccc54)cc3)c3cccc(-c4cccc5c4oc4ccccc45)c3)cc2-c2cccc3c2oc2ccccc23)cc1 ; CC1(C)c2ccccc2-c2ccc(-c3ccc(N(c4ccc(-n5c6ccccc6c6cnccc65)cc4)c4ccc(-c5ccccc5)c(-c5cccc6c5oc5ccccc56)c4)cc3)cc21 ; CC1(C)c2ccccc2-c2ccc(-c3ccc(N(c4ccc(-n5c6ccccc6c6cnccc65)cc4)c4ccc(-c5ccccc5)c(-c5cccc6c5oc5ccccc56)c4)cc3)cc21]

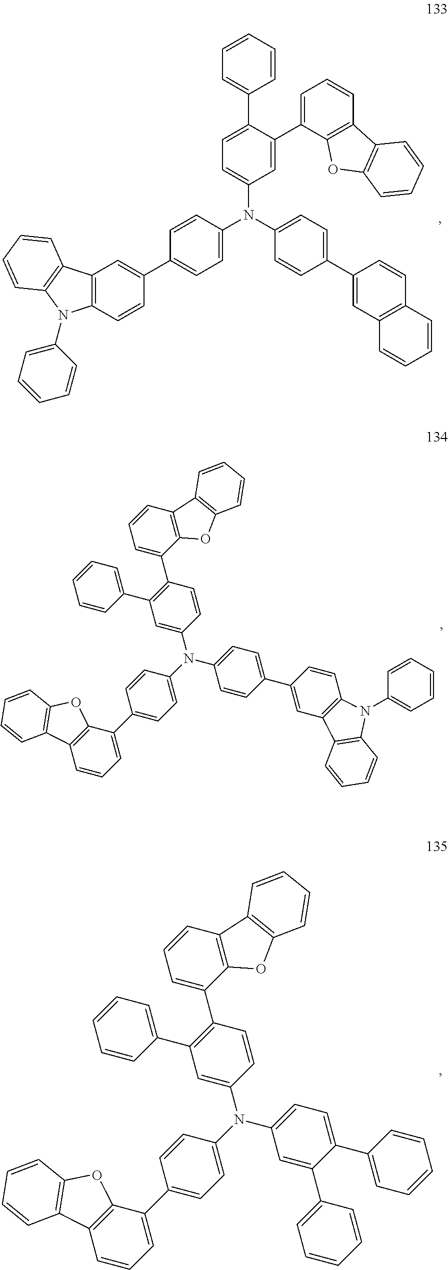 [SMILES: c1ccc(-c2ccc(N(c3ccc(-c4ccc5ccccc5c4)cc3)c3ccc(-c4ccc5c(c4)c4ccccc4n5-c4ccccc4)cc3)cc2-c2cccc3c2oc2ccccc23)cc1 ; c1ccc(-c2cc(N(c3ccc(-c4ccc5c(c4)c4ccccc4n5-c4ccccc4)cc3)c3ccc(-c4cccc5c4oc4ccccc45)cc3)ccc2-c2cccc3c2oc2ccccc23)cc1 ; c1ccc(-c2ccc(N(c3ccc(-c4cccc5c4oc4ccccc45)cc3)c3ccc(-c4cccc5c4oc4ccccc45)c(-c4ccccc4)c3)cc2-c2ccccc2)cc1]

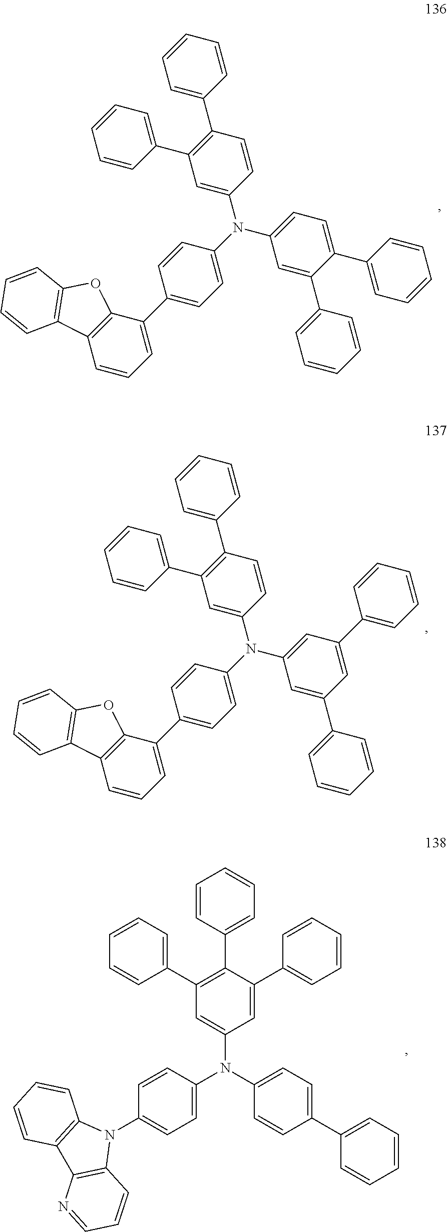 [SMILES: c1ccc(-c2ccc(N(c3ccc(-c4cccc5c4oc4ccccc45)cc3)c3ccc(-c4ccccc4)c(-c4ccccc4)c3)cc2-c2ccccc2)cc1 ; c1ccc(-c2cc(-c3ccccc3)cc(N(c3ccc(-c4cccc5c4oc4ccccc45)cc3)c3ccc(-c4ccccc4)c(-c4ccccc4)c3)c2)cc1 ; c1ccc(-c2ccc(N(c3ccc(-n4c5ccccc5c5ncccc54)cc3)c3cc(-c4ccccc4)c(-c4ccccc4)c(-c4ccccc4)c3)cc2)cc1]

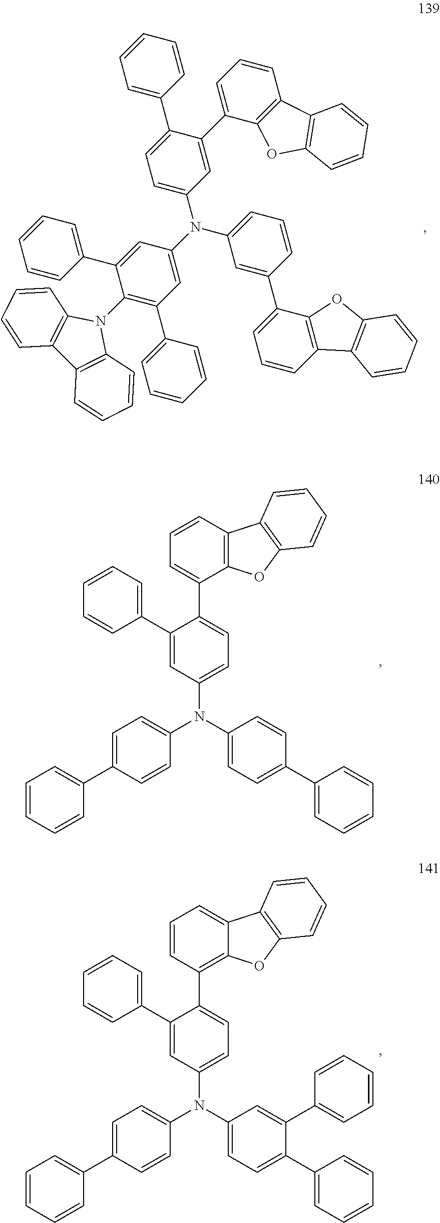 [SMILES: c1ccc(-c2ccc(N(c3cccc(-c4cccc5c4oc4ccccc45)c3)c3cc(-c4ccccc4)c(-n4c5ccccc5c5ccccc54)c(-c4ccccc4)c3)cc2-c2cccc3c2oc2ccccc23)cc1 ; c1ccc(-c2ccc(N(c3ccc(-c4ccccc4)cc3)c3ccc(-c4cccc5c4oc4ccccc45)c(-c4ccccc4)c3)cc2)cc1 ; c1ccc(-c2ccc(N(c3ccc(-c4ccccc4)c(-c4ccccc4)c3)c3ccc(-c4cccc5c4oc4ccccc45)c(-c4ccccc4)c3)cc2)cc1]

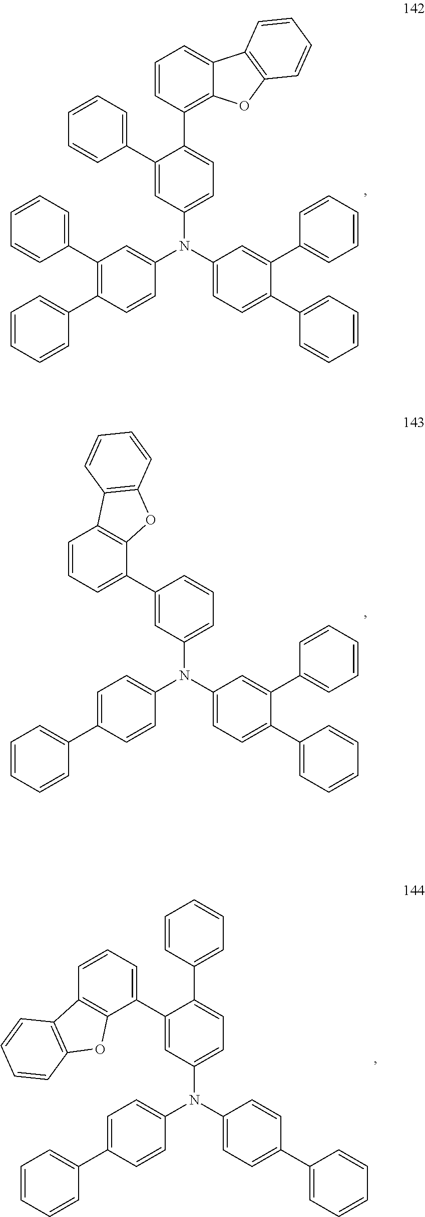 [SMILES: c1ccc(-c2ccc(N(c3ccc(-c4ccccc4)c(-c4ccccc4)c3)c3ccc(-c4cccc5c4oc4ccccc45)c(-c4ccccc4)c3)cc2-c2ccccc2)cc1 ; c1ccc(-c2ccc(N(c3cccc(-c4cccc5c4oc4ccccc45)c3)c3ccc(-c4ccccc4)c(-c4ccccc4)c3)cc2)cc1 ; c1ccc(-c2ccc(N(c3ccc(-c4ccccc4)cc3)c3ccc(-c4ccccc4)c(-c4cccc5c4oc4ccccc45)c3)cc2)cc1]

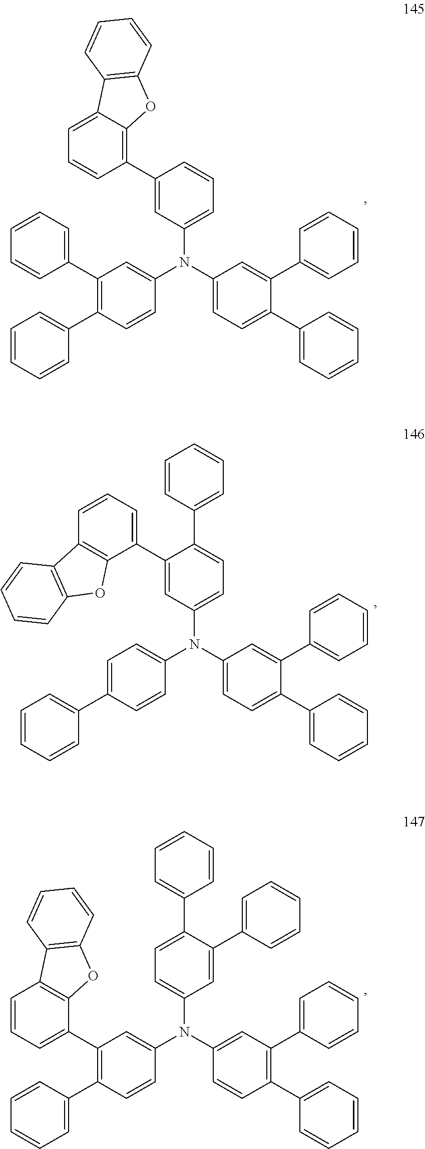 [SMILES: c1ccc(-c2ccc(N(c3cccc(-c4cccc5c4oc4ccccc45)c3)c3ccc(-c4ccccc4)c(-c4ccccc4)c3)cc2-c2ccccc2)cc1 ; c1ccc(-c2ccc(N(c3ccc(-c4ccccc4)c(-c4ccccc4)c3)c3ccc(-c4ccccc4)c(-c4cccc5c4oc4ccccc45)c3)cc2)cc1 ; c1ccc(-c2ccc(N(c3ccc(-c4ccccc4)c(-c4ccccc4)c3)c3ccc(-c4ccccc4)c(-c4cccc5c4oc4ccccc45)c3)cc2-c2ccccc2)cc1]

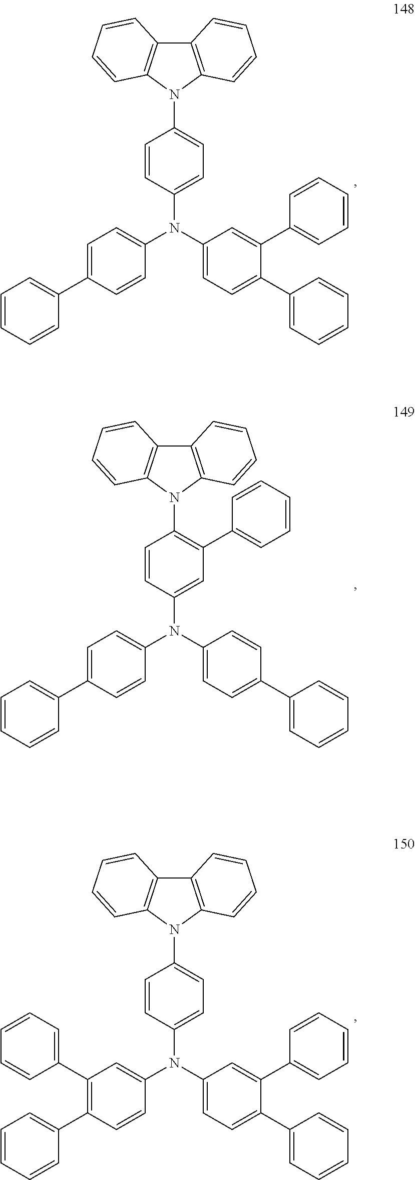 [SMILES: c1ccc(-c2ccc(N(c3ccc(-n4c5ccccc5c5ccccc54)cc3)c3ccc(-c4ccccc4)c(-c4ccccc4)c3)cc2)cc1 ; c1ccc(-c2ccc(N(c3ccc(-c4ccccc4)cc3)c3ccc(-n4c5ccccc5c5ccccc54)c(-c4ccccc4)c3)cc2)cc1 ; c1ccc(-c2ccc(N(c3ccc(-n4c5ccccc5c5ccccc54)cc3)c3ccc(-c4ccccc4)c(-c4ccccc4)c3)cc2-c2ccccc2)cc1]

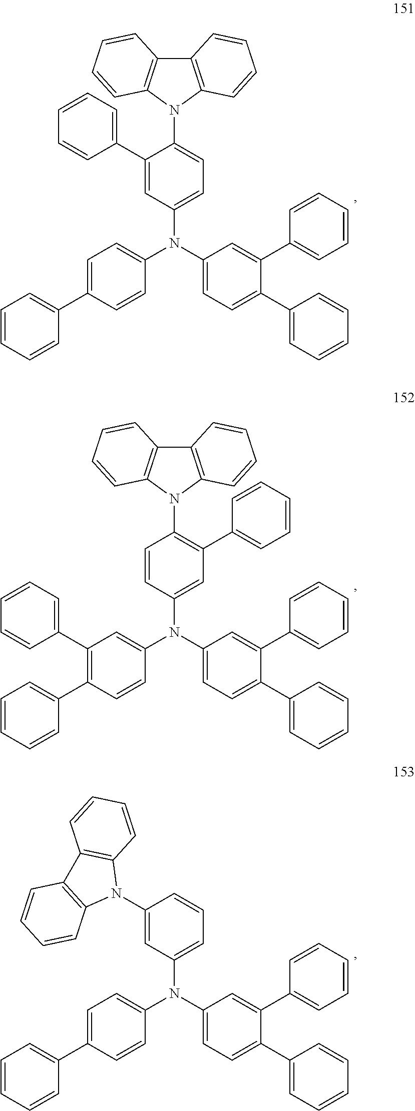 [SMILES: c1ccc(-c2ccc(N(c3ccc(-c4ccccc4)c(-c4ccccc4)c3)c3ccc(-n4c5ccccc5c5ccccc54)c(-c4ccccc4)c3)cc2)cc1 ; c1ccc(-c2ccc(N(c3ccc(-c4ccccc4)c(-c4ccccc4)c3)c3ccc(-n4c5ccccc5c5ccccc54)c(-c4ccccc4)c3)cc2-c2ccccc2)cc1 ; c1ccc(-c2ccc(N(c3cccc(-n4c5ccccc5c5ccccc54)c3)c3ccc(-c4ccccc4)c(-c4ccccc4)c3)cc2)cc1]

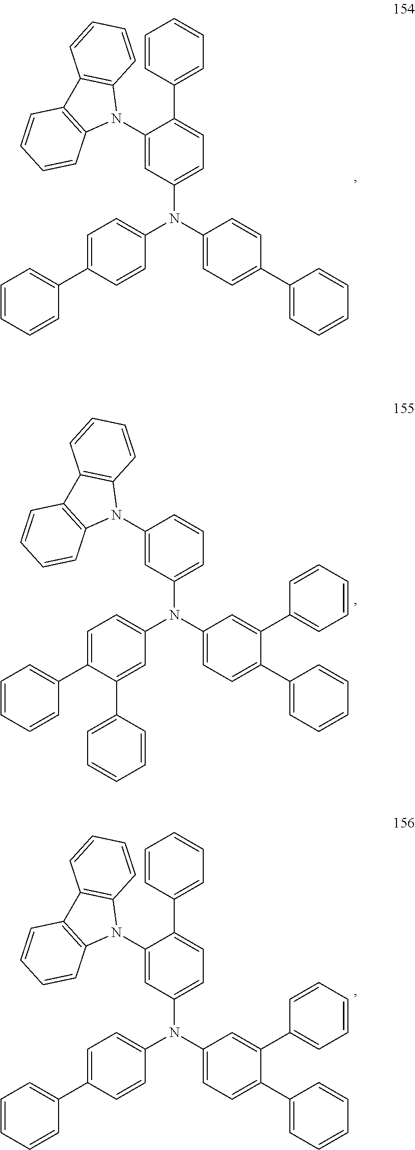 [SMILES: c1ccc(-c2ccc(N(c3ccc(-c4ccccc4)cc3)c3ccc(-c4ccccc4)c(-n4c5ccccc5c5ccccc54)c3)cc2)cc1 ; c1ccc(-c2ccc(N(c3cccc(-n4c5ccccc5c5ccccc54)c3)c3ccc(-c4ccccc4)c(-c4ccccc4)c3)cc2-c2ccccc2)cc1 ; c1ccc(-c2ccc(N(c3ccc(-c4ccccc4)c(-c4ccccc4)c3)c3ccc(-c4ccccc4)c(-n4c5ccccc5c5ccccc54)c3)cc2)cc1]

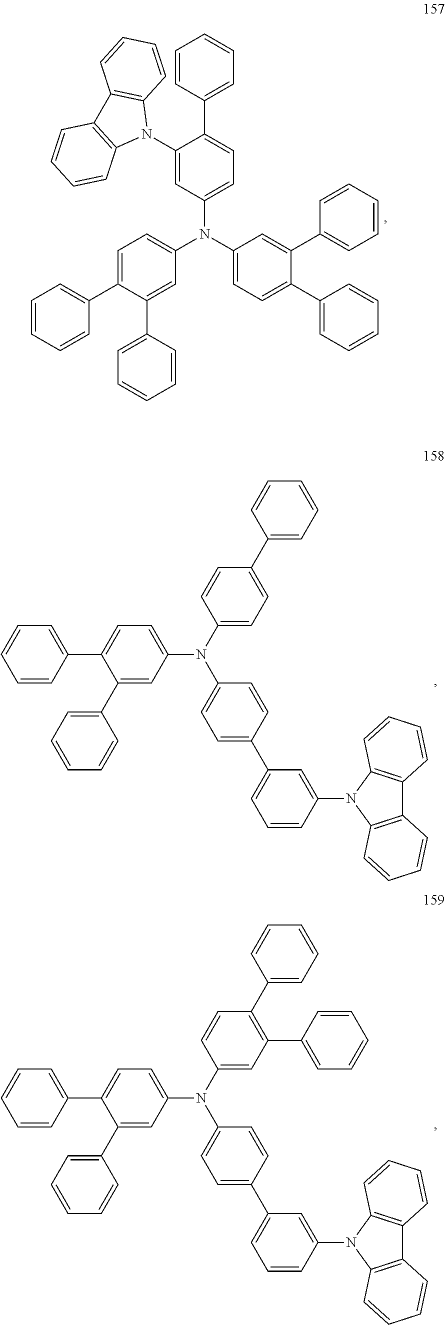 [SMILES: c1ccc(-c2ccc(N(c3ccc(-c4ccccc4)c(-c4ccccc4)c3)c3ccc(-c4ccccc4)c(-n4c5ccccc5c5ccccc54)c3)cc2-c2ccccc2)cc1 ; c1ccc(-c2ccc(N(c3ccc(-c4cccc(-n5c6ccccc6c6ccccc65)c4)cc3)c3ccc(-c4ccccc4)c(-c4ccccc4)c3)cc2)cc1 ; c1ccc(-c2ccc(N(c3ccc(-c4cccc(-n5c6ccccc6c6ccccc65)c4)cc3)c3ccc(-c4ccccc4)c(-c4ccccc4)c3)cc2-c2ccccc2)cc1]

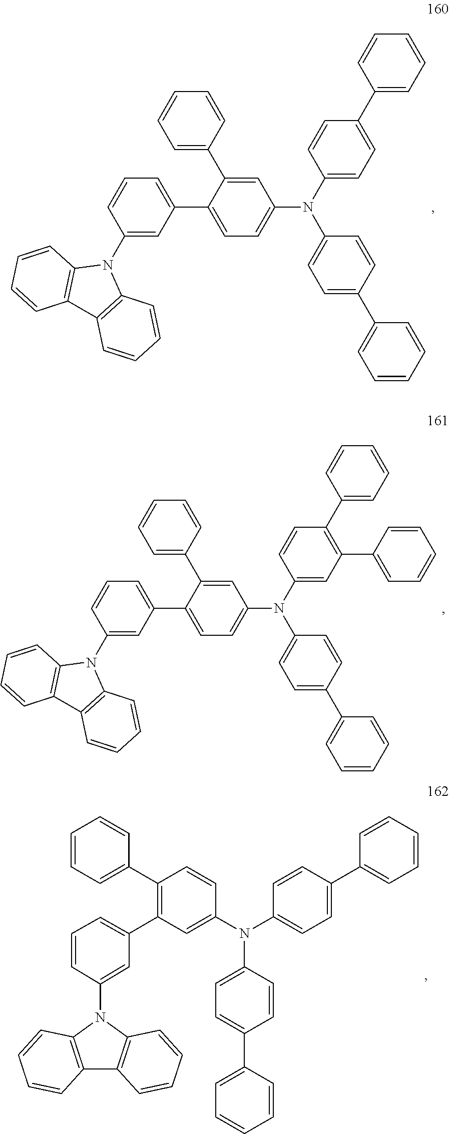 [SMILES: c1ccc(-c2ccc(N(c3ccc(-c4ccccc4)cc3)c3ccc(-c4cccc(-n5c6ccccc6c6ccccc65)c4)c(-c4ccccc4)c3)cc2)cc1 ; c1ccc(-c2ccc(N(c3ccc(-c4ccccc4)c(-c4ccccc4)c3)c3ccc(-c4cccc(-n5c6ccccc6c6ccccc65)c4)c(-c4ccccc4)c3)cc2)cc1 ; c1ccc(-c2ccc(N(c3ccc(-c4ccccc4)cc3)c3ccc(-c4ccccc4)c(-c4cccc(-n5c6ccccc6c6ccccc65)c4)c3)cc2)cc1]

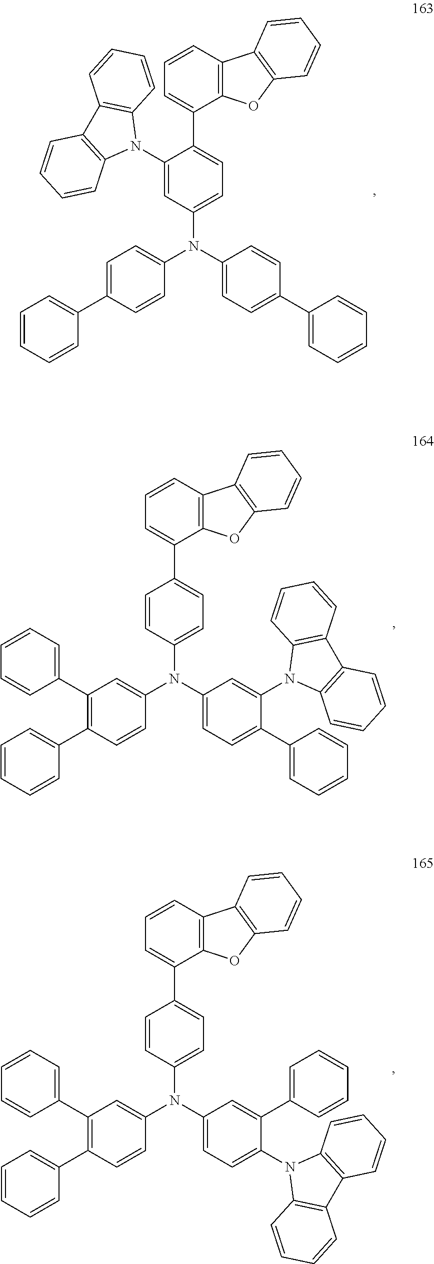 [SMILES: c1ccc(-c2ccc(N(c3ccc(-c4ccccc4)cc3)c3ccc(-c4cccc5c4oc4ccccc45)c(-n4c5ccccc5c5ccccc54)c3)cc2)cc1 ; c1ccc(-c2ccc(N(c3ccc(-c4cccc5c4oc4ccccc45)cc3)c3ccc(-c4ccccc4)c(-n4c5ccccc5c5ccccc54)c3)cc2-c2ccccc2)cc1 ; c1ccc(-c2ccc(N(c3ccc(-c4cccc5c4oc4ccccc45)cc3)c3ccc(-n4c5ccccc5c5ccccc54)c(-c4ccccc4)c3)cc2-c2ccccc2)cc1]

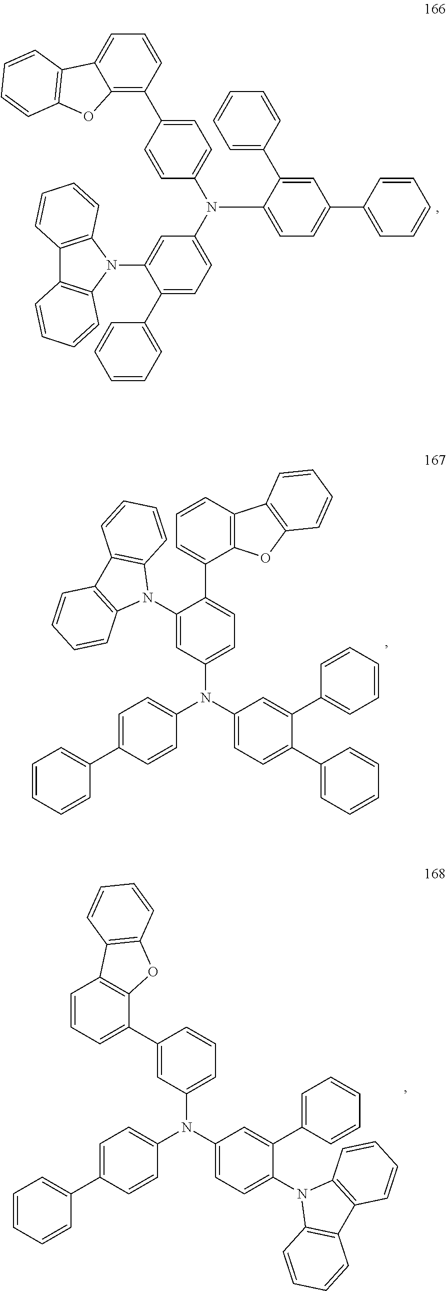 [SMILES: c1ccc(-c2ccc(N(c3ccc(-c4cccc5c4oc4ccccc45)cc3)c3ccc(-c4ccccc4)c(-n4c5ccccc5c5ccccc54)c3)c(-c3ccccc3)c2)cc1 ; c1ccc(-c2ccc(N(c3ccc(-c4ccccc4)c(-c4ccccc4)c3)c3ccc(-c4cccc5c4oc4ccccc45)c(-n4c5ccccc5c5ccccc54)c3)cc2)cc1 ; c1ccc(-c2ccc(N(c3cccc(-c4cccc5c4oc4ccccc45)c3)c3ccc(-n4c5ccccc5c5ccccc54)c(-c4ccccc4)c3)cc2)cc1]

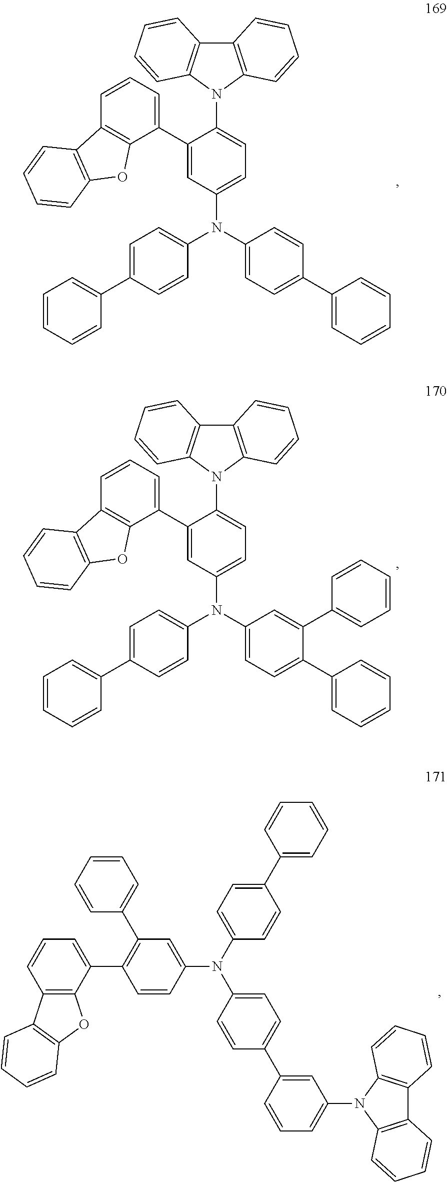 [SMILES: c1ccc(-c2ccc(N(c3ccc(-c4ccccc4)cc3)c3ccc(-n4c5ccccc5c5ccccc54)c(-c4cccc5c4oc4ccccc45)c3)cc2)cc1 ; c1ccc(-c2ccc(N(c3ccc(-c4ccccc4)c(-c4ccccc4)c3)c3ccc(-n4c5ccccc5c5ccccc54)c(-c4cccc5c4oc4ccccc45)c3)cc2)cc1 ; c1ccc(-c2ccc(N(c3ccc(-c4cccc(-n5c6ccccc6c6ccccc65)c4)cc3)c3ccc(-c4cccc5c4oc4ccccc45)c(-c4ccccc4)c3)cc2)cc1]

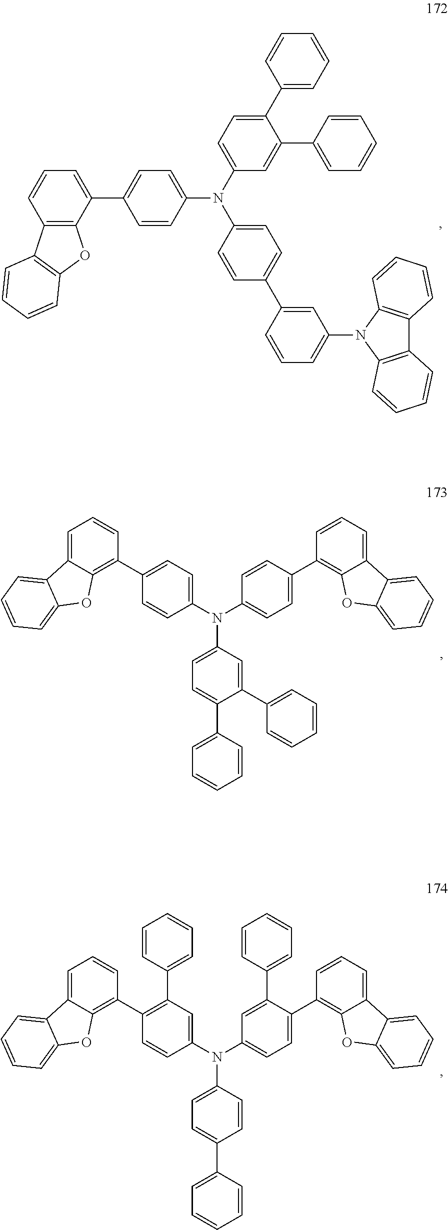 [SMILES: c1ccc(-c2ccc(N(c3ccc(-c4cccc(-n5c6ccccc6c6ccccc65)c4)cc3)c3ccc(-c4cccc5c4oc4ccccc45)cc3)cc2-c2ccccc2)cc1 ; c1ccc(-c2ccc(N(c3ccc(-c4cccc5c4oc4ccccc45)cc3)c3ccc(-c4cccc5c4oc4ccccc45)cc3)cc2-c2ccccc2)cc1 ; c1ccc(-c2ccc(N(c3ccc(-c4cccc5c4oc4ccccc45)c(-c4ccccc4)c3)c3ccc(-c4cccc5c4oc4ccccc45)c(-c4ccccc4)c3)cc2)cc1]

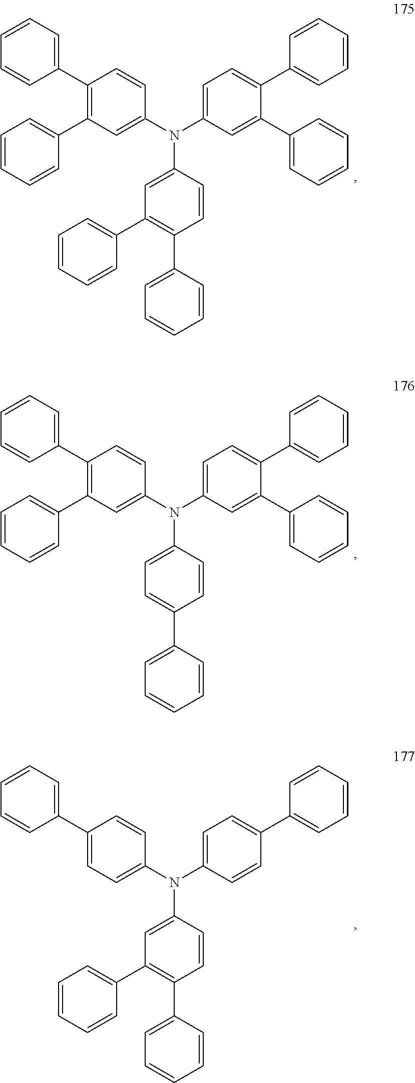 [SMILES: c1ccc(-c2ccc(N(c3ccc(-c4ccccc4)c(-c4ccccc4)c3)c3ccc(-c4ccccc4)c(-c4ccccc4)c3)cc2-c2ccccc2)cc1 ; c1ccc(-c2ccc(N(c3ccc(-c4ccccc4)c(-c4ccccc4)c3)c3ccc(-c4ccccc4)c(-c4ccccc4)c3)cc2)cc1 ; c1ccc(-c2ccc(N(c3ccc(-c4ccccc4)cc3)c3ccc(-c4ccccc4)c(-c4ccccc4)c3)cc2)cc1]

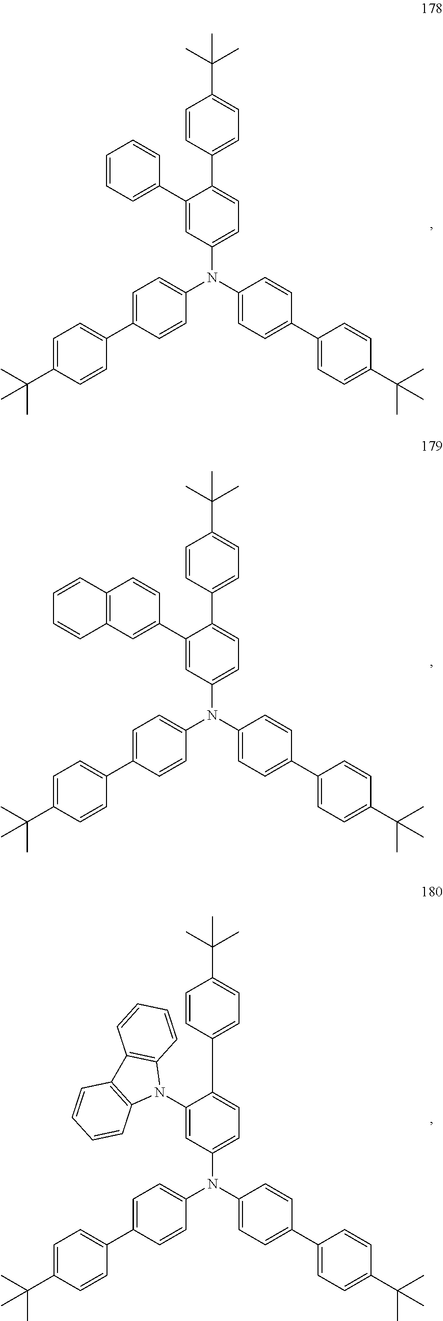

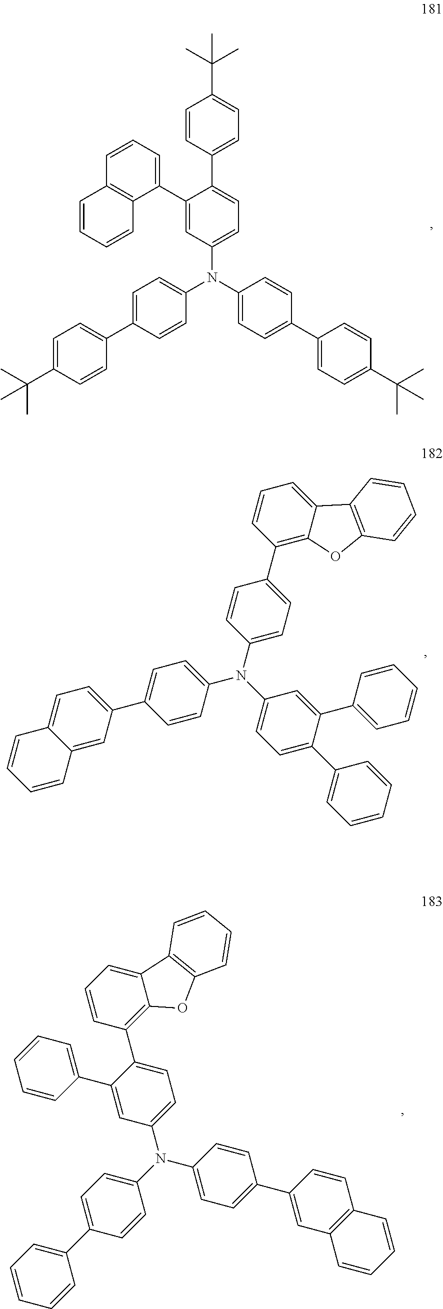 [SMILES: CC(C)(C)c1ccc(-c2ccc(N(c3ccc(-c4ccc(C(C)(C)C)cc4)cc3)c3ccc(-c4ccc(C(C)(C)C)cc4)c(-c4cccc5ccccc45)c3)cc2)cc1 ; c1ccc(-c2ccc(N(c3ccc(-c4ccc5ccccc5c4)cc3)c3ccc(-c4cccc5c4oc4ccccc45)cc3)cc2-c2ccccc2)cc1 ; c1ccc(-c2ccc(N(c3ccc(-c4ccc5ccccc5c4)cc3)c3ccc(-c4cccc5c4oc4ccccc45)c(-c4ccccc4)c3)cc2)cc1]

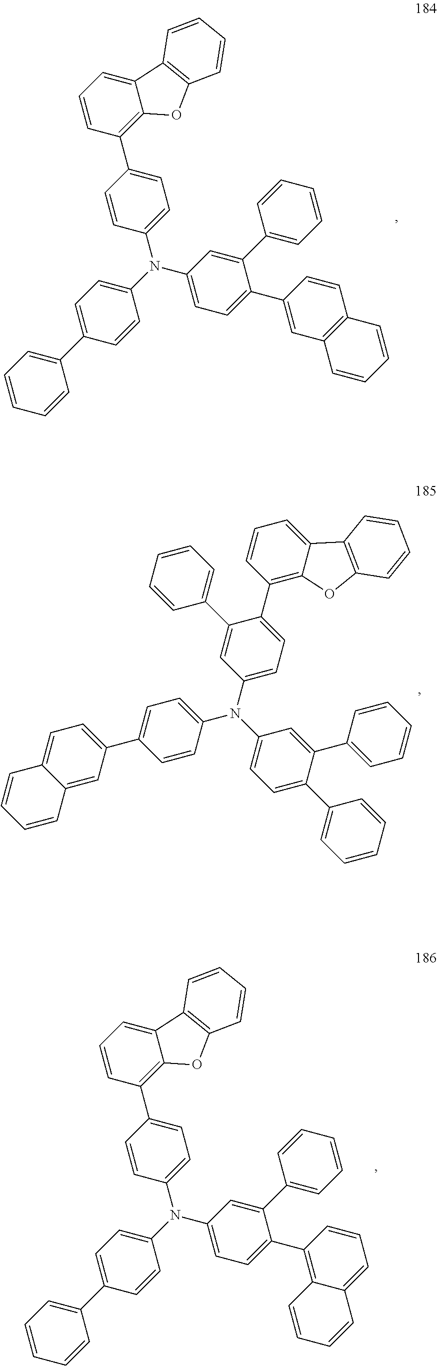 [SMILES: c1ccc(-c2ccc(N(c3ccc(-c4cccc5c4oc4ccccc45)cc3)c3ccc(-c4ccc5ccccc5c4)c(-c4ccccc4)c3)cc2)cc1 ; c1ccc(-c2ccc(N(c3ccc(-c4ccc5ccccc5c4)cc3)c3ccc(-c4cccc5c4oc4ccccc45)c(-c4ccccc4)c3)cc2-c2ccccc2)cc1 ; c1ccc(-c2ccc(N(c3ccc(-c4cccc5c4oc4ccccc45)cc3)c3ccc(-c4cccc5ccccc45)c(-c4ccccc4)c3)cc2)cc1]

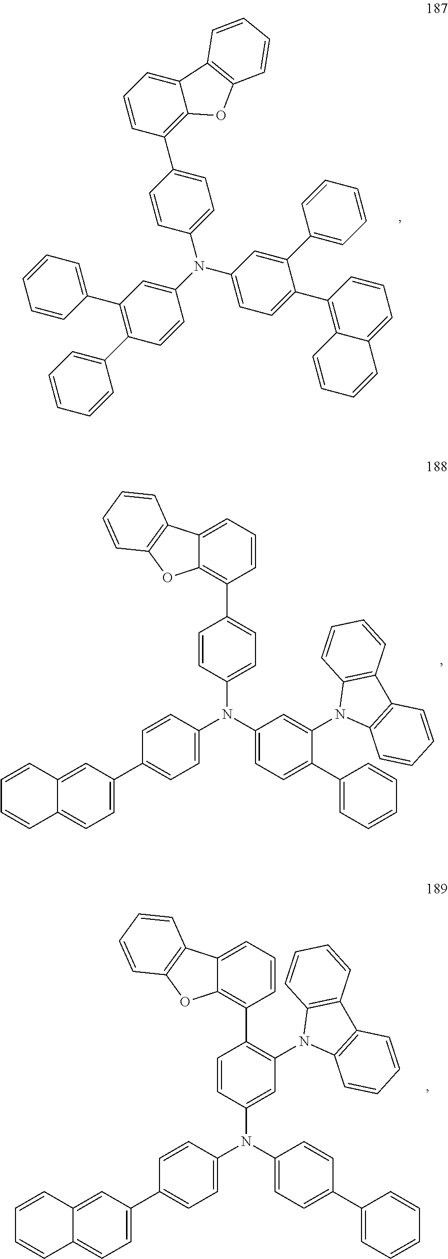 [SMILES: c1ccc(-c2ccc(N(c3ccc(-c4cccc5c4oc4ccccc45)cc3)c3ccc(-c4cccc5ccccc45)c(-c4ccccc4)c3)cc2-c2ccccc2)cc1 ; c1ccc(-c2ccc(N(c3ccc(-c4ccc5ccccc5c4)cc3)c3ccc(-c4cccc5c4oc4ccccc45)cc3)cc2-n2c3ccccc3c3ccccc32)cc1 ; c1ccc(-c2ccc(N(c3ccc(-c4ccc5ccccc5c4)cc3)c3ccc(-c4cccc5c4oc4ccccc45)c(-n4c5ccccc5c5ccccc54)c3)cc2)cc1]

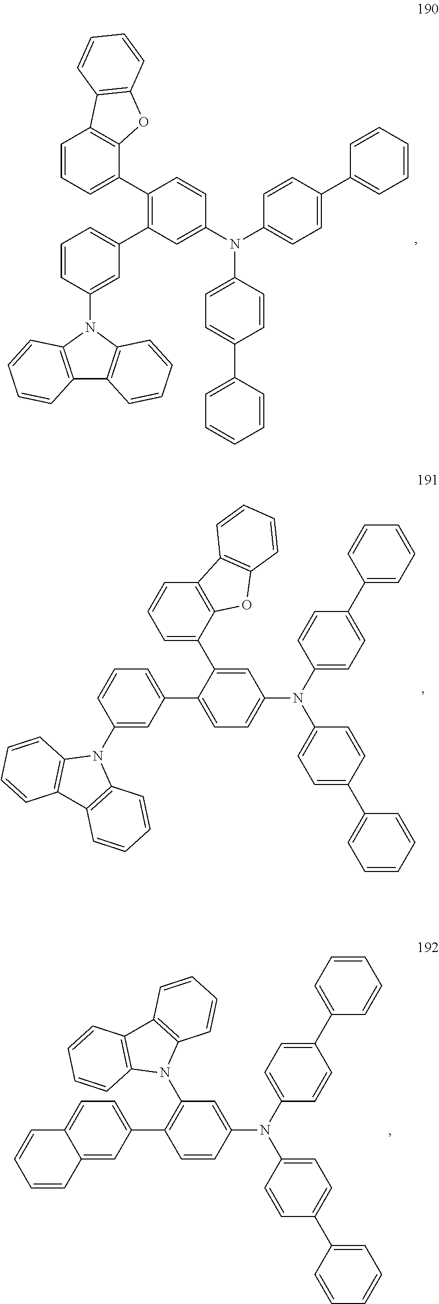 [SMILES: c1ccc(-c2ccc(N(c3ccc(-c4ccccc4)cc3)c3ccc(-c4cccc5c4oc4ccccc45)c(-c4cccc(-n5c6ccccc6c6ccccc65)c4)c3)cc2)cc1 ; c1ccc(-c2ccc(N(c3ccc(-c4ccccc4)cc3)c3ccc(-c4cccc(-n5c6ccccc6c6ccccc65)c4)c(-c4cccc5c4oc4ccccc45)c3)cc2)cc1 ; c1ccc(-c2ccc(N(c3ccc(-c4ccccc4)cc3)c3ccc(-c4ccc5ccccc5c4)c(-n4c5ccccc5c5ccccc54)c3)cc2)cc1]

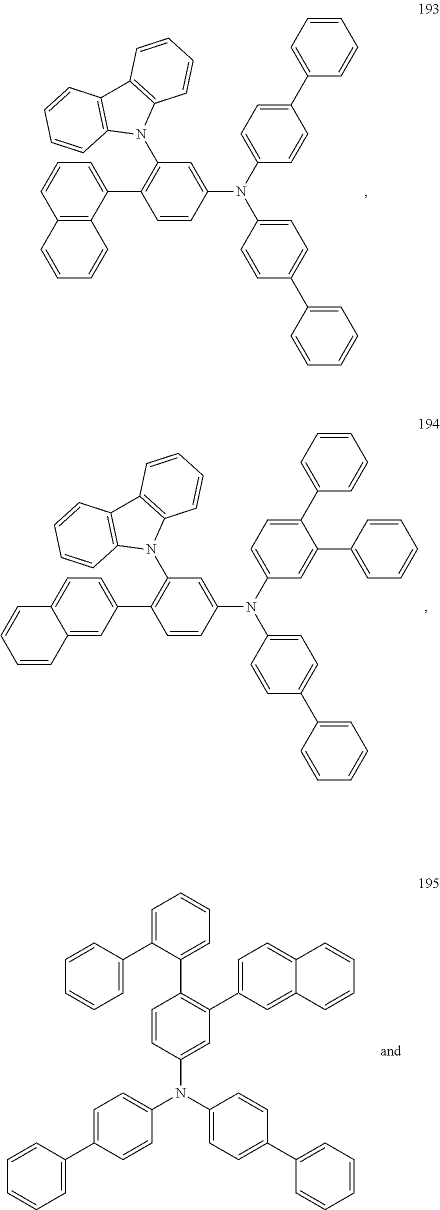 [SMILES: c1ccc(-c2ccc(N(c3ccc(-c4ccccc4)cc3)c3ccc(-c4cccc5ccccc45)c(-n4c5ccccc5c5ccccc54)c3)cc2)cc1 ; c1ccc(-c2ccc(N(c3ccc(-c4ccccc4)c(-c4ccccc4)c3)c3ccc(-c4ccc5ccccc5c4)c(-n4c5ccccc5c5ccccc54)c3)cc2)cc1 ; c1ccc(-c2ccc(N(c3ccc(-c4ccccc4)cc3)c3ccc(-c4ccccc4-c4ccccc4)c(-c4ccc5ccccc5c4)c3)cc2)cc1]

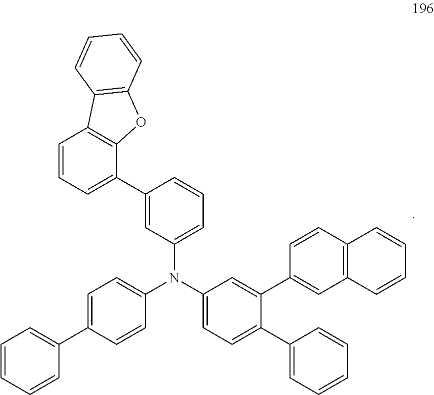 [SMILES: c1ccc(-c2ccc(N(c3cccc(-c4cccc5c4oc4ccccc45)c3)c3ccc(-c4ccccc4)c(-c4ccc5ccccc5c4)c3)cc2)cc1]

The present invention provides a method for preparing the compound with a core structure of triarylamine. The method involves the following reaction schemes:

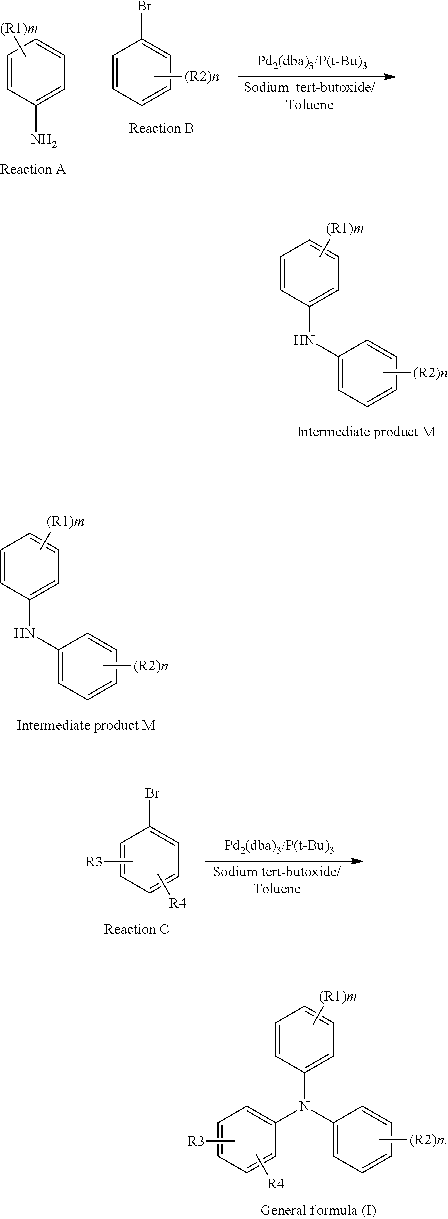

The method specifically comprises the following steps:

(1) With reactant A and reactant B as raw materials and toluene as solvent, Pd₂(dba)₃, P(t-Bu)₃ and sodium tert-butoxide are added to the reaction system under a nitrogen atmosphere, reacted at 95° C. to 110° C. for 10 to 24 h, and naturally cooled to room temperature; and the reaction solution is filtered, and the filtrate is subjected to rotary evaporation under reduced pressure and passed through a neutral silica gel column to obtain intermediate product M, wherein the toluene is used in an amount of 50 to 80 ml per g of the reactant A; the reactant A and the reactant B are present in a molar ratio of 1:0.8 to 1; Pd₂(dba)₃ and the reactant A are present in a molar ratio of 0.005 to 0.01:1; P(t-Bu)₃ and the reactant A are present in a molar ratio of 1.5 to 3.0:1; and sodium tert-butoxide and the reactant A are present in a molar ratio of 2 to 2.5:1; and

(2) With the intermediate product M obtained in step (1) and reactant C as raw materials and toluene as solvent, Pd₂(dba)₃, P(t-Bu)₃ and sodium tert-butoxide are added to the reaction system under a nitrogen atmosphere, reacted at 95° C. to 110° C. for 10 to 24 h, and naturally cooled to room temperature; and the reaction solution is filtered, and the filtrate is subjected to rotary evaporation under reduced pressure and passed through a neutral silica gel column to obtain a compound of general formula (1), wherein the toluene is used in an amount of 50 to 80 ml per g of the intermediate product M; the intermediate product M and the reactant C are present in a molar ratio of 1:1.0 to 1.5; the Pd₂(dba)₃ and the intermediate product M are present in a molar ratio of 0.005 to 0.01:1; the P(t-Bu)₃ and the intermediate product M are present in a molar ratio of 1.5 to 3.0:1; and the sodium tert-butoxide and intermediate product M are present in a molar ratio of 2 to 2.5:1.

The present invention provides an organic light-emitting device, wherein the organic light-emitting device contains at least one functional layer comprising the compound with a core structure of triarylamine.

The present invention provides an organic light-emitting device, wherein the compound with a core structure of triarylamine is used as a hole transporting layer or an electron blocking layer materials for making the organic light-emitting device.

The present invention provides a lighting or display element, wherein the element comprises the organic light-emitting device.

Unless stated otherwise, the C₁₋₂₀ alkyl as used in the present invention is preferably selected from the following groups: methyl, ethyl, n-propyl, isopropyl, n-butyl, isobutyl, 1-methylpropyl, tert-butyl, n-pentyl, isopentyl, 1-ethylpropyl, 1-methylbutyl, 2-methylbutyl, 3-methylbutyl, n-hexyl, 1-methylpentyl, 2-methylpentyl, 3-methylpentyl, 4-methylpentyl, 3,3-dimethylbutyl, 1-ethylbutyl and 2-ethylbutyl.

In the context of the present invention, the heteroaryl is a monocyclic or bicyclic aromatic heterocyclic (heteroaromatic) ring, which contains at most four identical or different ring heteroatoms selected from N, O and S, and is linked via a ring carbon atom or, if appropriate, via a ring nitrogen atom, and is preferably selected from the following groups: furyl, pyrrolyl, thienyl, pyrazolyl, imidazolyl, quinolyl, thiazolyl, oxazolyl, isoxazolyl, isothiazolyl, triazolyl, oxadiazolyl, thiadiazolyl, tetrazolyl, pyridyl, pyrimidinyl, pyridazinyl, pyrazinyl and triazinyl.

If the groups in the compound of the present invention are substituted, they may be mono-substituted or multi-substituted, unless specified otherwise.

The present invention has the following beneficial technical effects:

The compound of the present invention has a strong hole transport ability owing to the p-π conjugation effect existed therein. A high hole transport rate can lead to an improvement in the efficiency of the organic light-emitting device. The asymmetric triarylamine structure in the compound can reduce the crystallinity and the planarity of the molecules, and prevent the molecules from moving on the plane, thereby improving the thermal stability of the molecules.

The structure of the compound of the present invention makes the distribution of electrons and holes in the light-emitting layer more balanced. At an appropriate HOMO energy level, the compound with such a triarylamine structure which has a relatively high mobility and triplet energy level can improve the hole injection and transport performances. At a suitable HOMO energy level, the said compound also plays the role of electron blocking so as to improve the recombination efficiency of excitons in the light-emitting layer.

When the compound of the present invention is applied to OLED, by optimizing the structure of the device, a high stability of the film layers can be maintained, and the photoelectric properties and the service life of OLED can be effectively improved. The compound of the present invention has good application effects and industrialization prospects in organic light-emitting devices.

### BRIEF DESCRIPTION OF DRAWINGS

FIG. 1 is a schematic diagram of an organic light-emitting device in which the materials listed in the present invention are used;

1: a transparent substrate layer; 2: an ITO anode layer; 3: a hole injection layer; 4: a hole transporting layer; 5: an electron blocking layer; 6: a light-emitting layer; 7: a hole blocking/electron transporting layer; 8: an electron injection layer; 9: cathode reflective electrode layer.

FIG. 2 is a curve graph showing the efficiency of the organic light-emitting devices of the present invention measured at different temperatures.

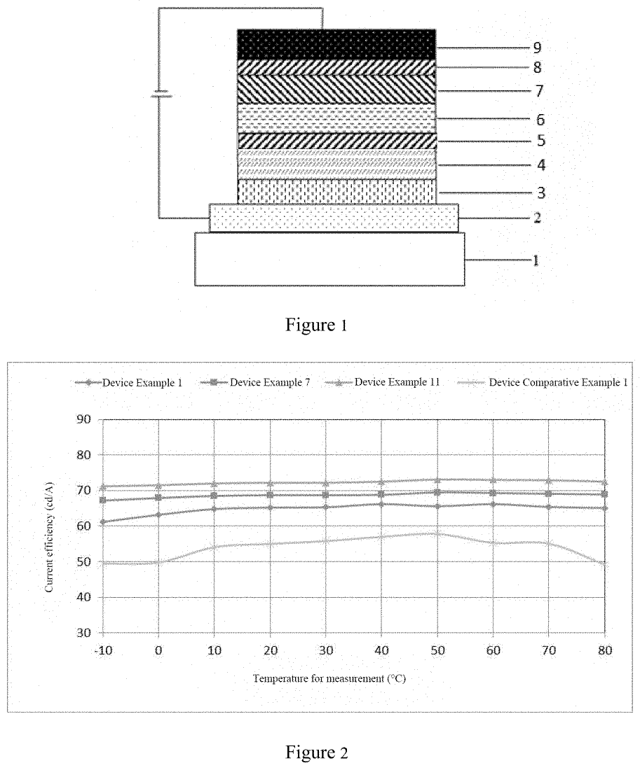

### DETAILED DESCRIPTION OF THE INVENTION

The present invention will be described in detail with reference to drawings and examples below.

### Example 1: Synthesis of Compound 1

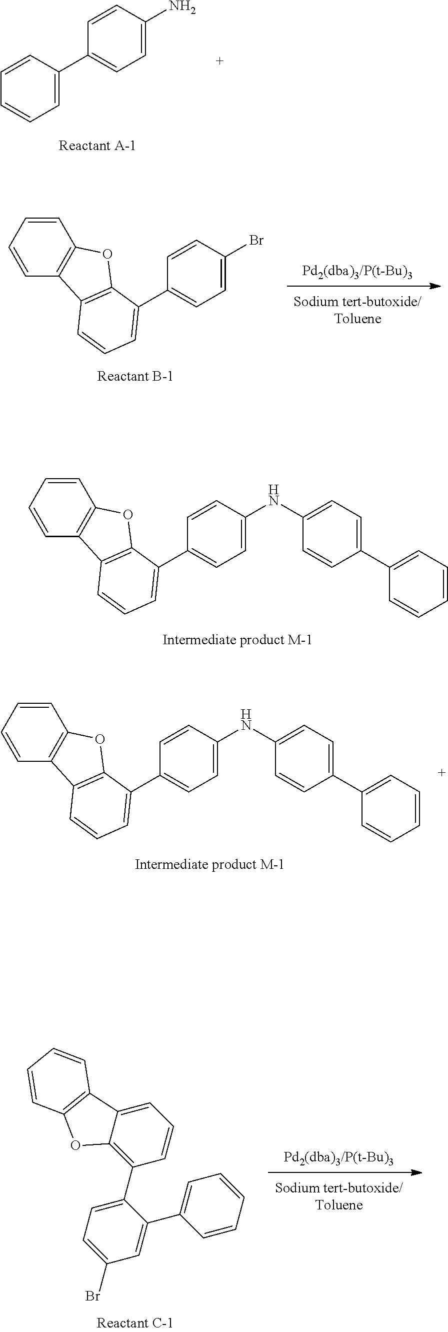 [SMILES: Nc1ccc(-c2ccccc2)cc1 ; Brc1ccc(-c2cccc3c2oc2ccccc23)cc1 ; c1ccc(-c2ccc(Nc3ccc(-c4cccc5c4oc4ccccc45)cc3)cc2)cc1 ; c1ccc(-c2ccc(Nc3ccc(-c4cccc5c4oc4ccccc45)cc3)cc2)cc1 ; Brc1ccc(-c2cccc3c2oc2ccccc23)c(-c2ccccc2)c1]

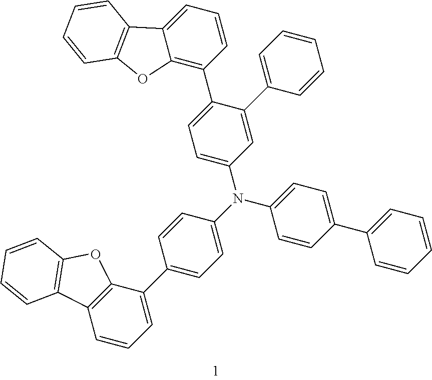 [SMILES: c1ccc(-c2ccc(N(c3ccc(-c4cccc5c4oc4ccccc45)cc3)c3ccc(-c4cccc5c4oc4ccccc45)c(-c4ccccc4)c3)cc2)cc1]

(1) Under the protection of nitrogen, 0.01 mol of reactant A-1, 0.01 mol of reactant B-1, and 150 ml of toluene were added to a 250 ml three-necked flask, stirred and mixed. Then, 5×10⁻⁵ mol Pd₂(dba)₃, 5×10⁻⁵ mol P(t-Bu)₃, and 0.03 mol sodium tert-butoxide were added, heated to 105° C., and reacted under reflux for 24 h. Samples were taken and applied onto a plate, which showed that no bromine was left and the reaction was complete. The reaction mixture was naturally cooled to room temperature, and filtered. The filtrate was subjected to rotary evaporation until no more distillate was observed, and passed through a neutral silica gel column to obtain intermediate product M-1, with a HPLC purity of 99.66% and a yield of 86.8%. HRMS(EI): the molecular weight of the material=410.1539; the measured molecular weight of material=410.1529.

(2) Under the protection of nitrogen, 0.01 mol of the intermediate product M-1 obtained in step (1), 0.01 mol reactant C and 150 ml of toluene were added to a 250 ml three-necked flask, stirred and mixed. Then, 5×10⁻⁵ mol Pd₂(dba)₃, 5×10⁻⁵ mol P(t-Bu)₃, and 0.03 mol sodium tert-butoxide were added, heated to 105° C., and reacted under reflux for 24 h. Samples were taken and applied onto a plate, which showed that the reaction was complete. The reaction mixture was naturally cooled to room temperature, and filtered. The filtrate was subjected to rotary evaporation until no more distillate was observed, and passed through a neutral silica gel column to obtain intermediate product M-1, with a HPLC purity of 99.25% and a yield of 84.5%. HRMS(EI): the molecular weight of the material=729.2668; the measured molecular weight of material=729.2649.

The following compounds were synthesized by repeating the preparation process in Example 1, except that reactant A, reactant B and reactant C listed in Table 1 below were used.

TABLE 1

| No. | Reactants A | Reactants B | Reactants C | Target compounds | Test results |
|---|---|---|---|---|---|
| Example 2 | 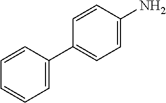 [SMILES: Nc1ccc(-c2ccccc2)cc1] | 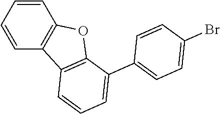 [SMILES: Brc1ccc(-c2cccc3c2oc2ccccc23)cc1] | 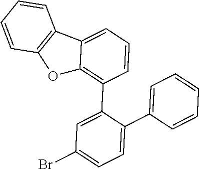 [SMILES: Brc1ccc(-c2ccccc2)c(-c2cccc3c2oc2ccccc23)c1] | 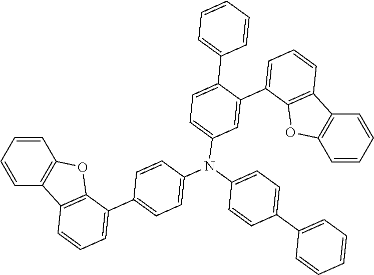 [SMILES: c1ccc(-c2ccc(N(c3ccc(-c4cccc5c4oc4ccccc45)cc3)c3ccc(-c4ccccc4)c(-c4cccc5c4oc4ccccc45)c3)cc2)cc1] | HPLC purity 98.20%, yield 68.5%; HRMS(EI): calculated value = 729.2668, measured value = 729.2658; |
| | Reactant A-1 | Reactant B-1 | Reactant C-2 | 6 | |
| Example 3 | 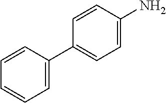 [SMILES: Nc1ccc(-c2ccccc2)cc1] | 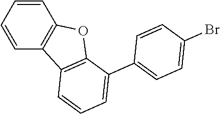 [SMILES: Brc1ccc(-c2cccc3c2oc2ccccc23)cc1] | 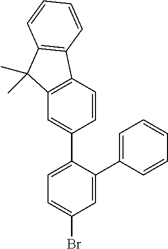 [SMILES: CC1(C)c2ccccc2-c2ccc(-c3ccc(Br)cc3-c3ccccc3)cc21] | 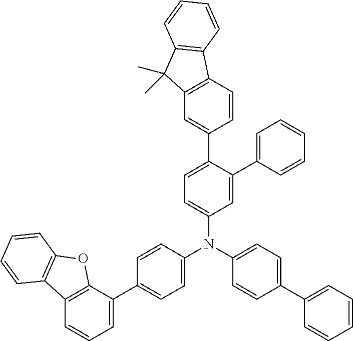 [SMILES: CC1(C)c2ccccc2-c2ccc(-c3ccc(N(c4ccc(-c5ccccc5)cc4)c4ccc(-c5cccc6c5oc5ccccc56)cc4)cc3-c3ccccc3)cc21] | HPLC purity 97.52%, yield 75.5%; HRMS(EI): calculated value = 755.3188, measured value = 755.3170; |
| | Reactant A-1 | Reactant B-1 | Reactant C-3 | 11 | |
| Example 4 | 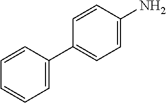 [SMILES: Nc1ccc(-c2ccccc2)cc1] | 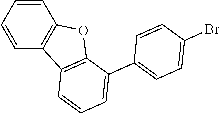 [SMILES: Brc1ccc(-c2cccc3c2oc2ccccc23)cc1] | 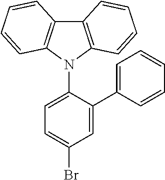 [SMILES: Brc1ccc(-n2c3ccccc3c3ccccc32)c(-c2ccccc2)c1] | 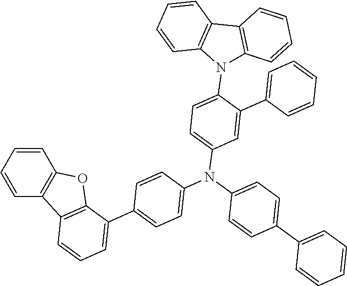 [SMILES: c1ccc(-c2ccc(N(c3ccc(-c4cccc5c4oc4ccccc45)cc3)c3ccc(-n4c5ccccc5c5ccccc54)c(-c4ccccc4)c3)cc2)cc1] | HPLC purity 97.65%, yield 80.5%; HRMS(EI): calculated value = 728.2828, measured value = 728.2805; |
| | Reactant A-1 | Reactant B-1 | Reactant C-3 | 21 | |
| Example 5 | 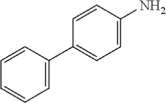 [SMILES: Nc1ccc(-c2ccccc2)cc1] | 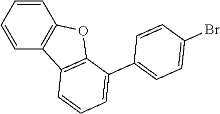 [SMILES: Brc1ccc(-c2cccc3c2oc2ccccc23)cc1] | 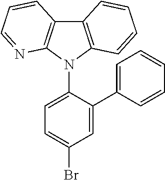 [SMILES: Brc1ccc(-n2c3ccccc3c3cccnc32)c(-c2ccccc2)c1] | 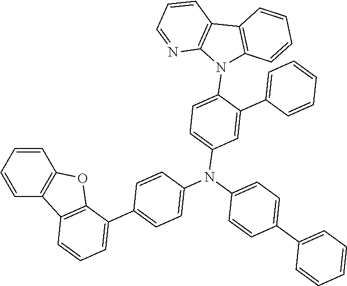 [SMILES: c1ccc(-c2ccc(N(c3ccc(-c4cccc5c4oc4ccccc45)cc3)c3ccc(-n4c5ccccc5c5cccnc54)c(-c4ccccc4)c3)cc2)cc1] | HPLC purity 99.25%, yield 68.0%; HRMS(EI): calculated value = 729.2780, measured value = 729.2721; |
| | Reactant A-1 | Reactant B-1 | Reactant C-4 | 26 | |
| Example 6 | 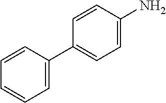 [SMILES: Nc1ccc(-c2ccccc2)cc1] | 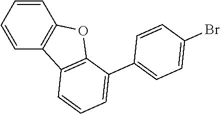 [SMILES: Brc1ccc(-c2cccc3c2oc2ccccc23)cc1] | 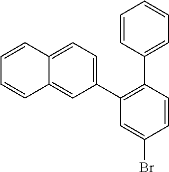 [SMILES: Brc1ccc(-c2ccccc2)c(-c2ccc3ccccc3c2)c1] | 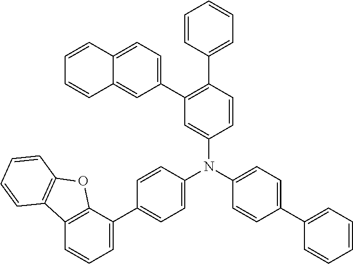 [SMILES: c1ccc(-c2ccc(N(c3ccc(-c4cccc5c4oc4ccccc45)cc3)c3ccc(-c4ccccc4)c(-c4ccc5ccccc5c4)c3)cc2)cc1] | HPLC purity 98.15%, yield 75.0%; HRMS(EI): calculated value = 689.2719, measured value = 689.2735; |
| | Reactant A-1 | Reactant B-1 | Reactant C-5 | 42 | |
| Example 7 | 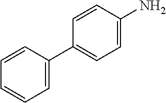 [SMILES: Nc1ccc(-c2ccccc2)cc1] | 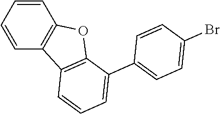 [SMILES: Brc1ccc(-c2cccc3c2oc2ccccc23)cc1] | 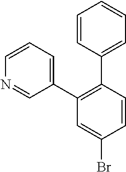 [SMILES: Brc1ccc(-c2ccccc2)c(-c2cccnc2)c1] | 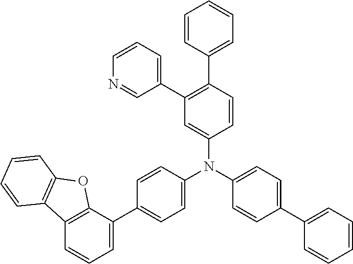 [SMILES: c1ccc(-c2ccc(N(c3ccc(-c4cccc5c4oc4ccccc45)cc3)c3ccc(-c4ccccc4)c(-c4cccnc4)c3)cc2)cc1] | HPLC purity 99.45%, yield 69.0%; HRMS(EI): calculated value = 640.2515, measured value = 640.2514; |
| | Reactant A-1 | Reactant B-1 | Reactant C-6 | 47 | |
| Example 8 | 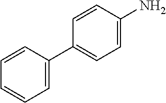 [SMILES: Nc1ccc(-c2ccccc2)cc1] | 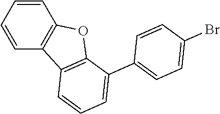 [SMILES: Brc1ccc(-c2cccc3c2oc2ccccc23)cc1] | 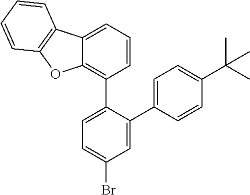 [SMILES: CC(C)(C)c1ccc(-c2cc(Br)ccc2-c2cccc3c2oc2ccccc23)cc1] | 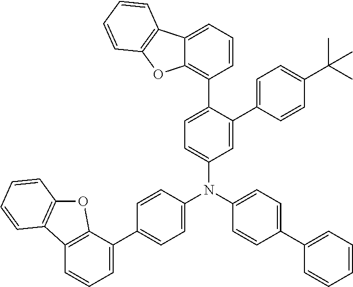 [SMILES: CC(C)(C)c1ccc(-c2cc(N(c3ccc(-c4ccccc4)cc3)c3ccc(-c4cccc5c4oc4ccccc45)cc3)ccc2-c2cccc3c2oc2ccccc23)cc1] | HPLC purity 98.25%, yield 69.0%; HRMS(EI): calculated value = 785.3294, measured value = 785.3221; |
| | Reactant A-1 | Reactant B-1 | Reactant C-6 | 54 | |
| Example 9 | 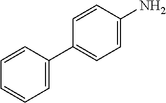 [SMILES: Nc1ccc(-c2ccccc2)cc1] | 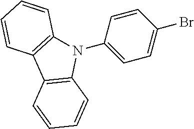 [SMILES: Brc1ccc(-n2c3ccccc3c3ccccc32)cc1] | 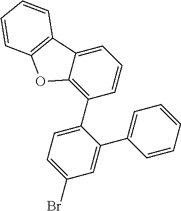 [SMILES: Brc1ccc(-c2cccc3c2oc2ccccc23)c(-c2ccccc2)c1] | 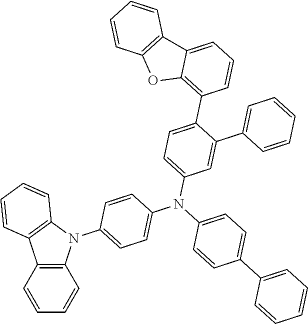 [SMILES: c1ccc(-c2ccc(N(c3ccc(-n4c5ccccc5c5ccccc54)cc3)c3ccc(-c4cccc5c4oc4ccccc45)c(-c4ccccc4)c3)cc2)cc1] | HPLC purity 99.00%, yield 72.5%; HRMS(EI): calculated value = 728.2828, measured value = 728.2823; |
| | Reactant A-1 | Reactant B-2 | Reactant C-1 | 62 | |
| Example 10 | 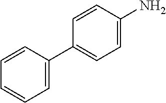 [SMILES: Nc1ccc(-c2ccccc2)cc1] | 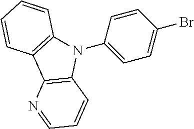 [SMILES: Brc1ccc(-n2c3ccccc3c3ncccc32)cc1] | 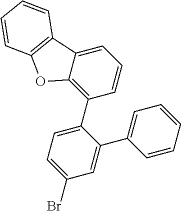 [SMILES: Brc1ccc(-c2cccc3c2oc2ccccc23)c(-c2ccccc2)c1] | 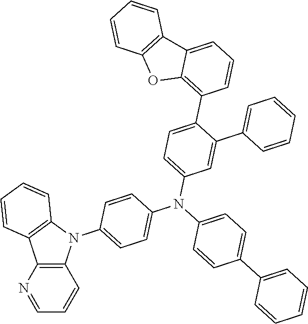 [SMILES: c1ccc(-c2ccc(N(c3ccc(-n4c5ccccc5c5ncccc54)cc3)c3ccc(-c4cccc5c4oc4ccccc45)c(-c4ccccc4)c3)cc2)cc1] | HPLC purity 98.20%, yield 65.8%; HRMS(EI): calculated value = 729.2780, measured value = 729.2755; |
| | Reactant A-1 | Reactant B-3 | Reactant C-1 | 68 | |
| Example 11 | 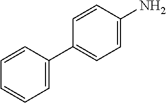 [SMILES: Nc1ccc(-c2ccccc2)cc1] | 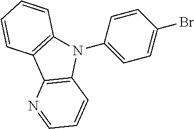 [SMILES: Brc1ccc(-n2c3ccccc3c3ncccc32)cc1] | 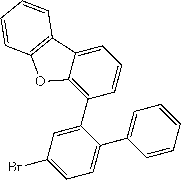 [SMILES: Brc1ccc(-c2ccccc2)c(-c2cccc3c2oc2ccccc23)c1] | 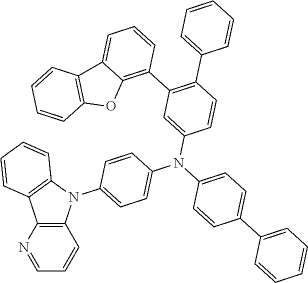 [SMILES: c1ccc(-c2ccc(N(c3ccc(-n4c5ccccc5c5ncccc54)cc3)c3ccc(-c4ccccc4)c(-c4cccc5c4oc4ccccc45)c3)cc2)cc1] | HPLC purity 99.50%, yield 75.5%; HRMS(EI): calculated value = 729.2780, measured value = 729.2719; |
| | Reactant A-1 | Reactant B-3 | Reactant C-2 | 78 | |
| Example 12 | 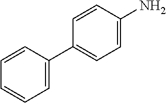 [SMILES: Nc1ccc(-c2ccccc2)cc1] | 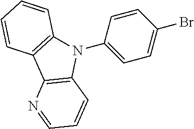 [SMILES: Brc1ccc(-n2c3ccccc3c3ncccc32)cc1] | 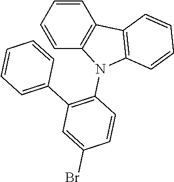 [SMILES: Brc1ccc(-n2c3ccccc3c3ccccc32)c(-c2ccccc2)c1] | 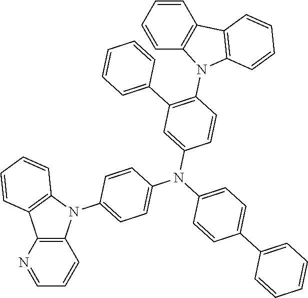 [SMILES: c1ccc(-c2ccc(N(c3ccc(-n4c5ccccc5c5ncccc54)cc3)c3ccc(-n4c5ccccc5c5ccccc54)c(-c4ccccc4)c3)cc2)cc1] | HPLC purity 99.05%, yield 72.5%; HRMS(EI): calculated value = 728.2940, measured value = 728.2961; |
| | Reactant A-1 | Reactant B-3 | Reactant C-3 | 90 | |
| Example 13 | 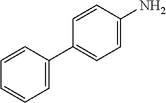 [SMILES: Nc1ccc(-c2ccccc2)cc1] | 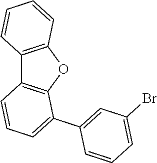 [SMILES: Brc1cccc(-c2cccc3c2oc2ccccc23)c1] | 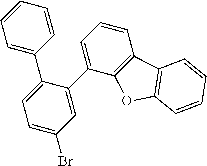 [SMILES: Brc1ccc(-c2ccccc2)c(-c2cccc3c2oc2ccccc23)c1] | 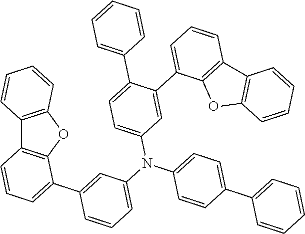 [SMILES: c1ccc(-c2ccc(N(c3cccc(-c4cccc5c4oc4ccccc45)c3)c3ccc(-c4ccccc4)c(-c4cccc5c4oc4ccccc45)c3)cc2)cc1] | HPLC purity 98.56%, yield 65.4%; HRMS(EI): calculated value = 729.2668, measured value = 729.2687; |
| | Reactant A-1 | Reactant B-4 | Reactant C-2 | 107 | |
| Example 14 | 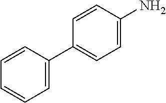 [SMILES: Nc1ccc(-c2ccccc2)cc1] | 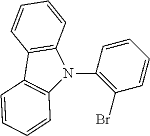 [SMILES: Brc1ccccc1-n1c2ccccc2c2ccccc21] | 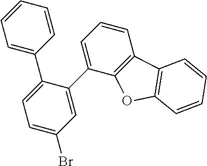 [SMILES: Brc1ccc(-c2ccccc2)c(-c2cccc3c2oc2ccccc23)c1] | 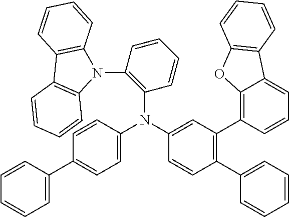 [SMILES: c1ccc(-c2ccc(N(c3ccc(-c4ccccc4)c(-c4cccc5c4oc4ccccc45)c3)c3ccccc3-n3c4ccccc4c4ccccc43)cc2)cc1] | HPLC purity 98.70%, yield 71.5%; HRMS(EI): calculated value = 728.2828, measured value = 728.2845; |
| | Reactant A-1 | Reactant B-5 | Reactant C-2 | 117 | |
| Example 15 | 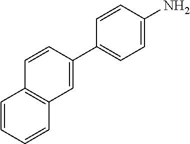 [SMILES: Nc1ccc(-c2ccc3ccccc3c2)cc1] | 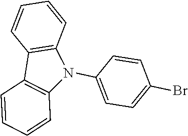 [SMILES: Brc1ccc(-n2c3ccccc3c3ccccc32)cc1] | 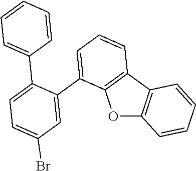 [SMILES: Brc1ccc(-c2ccccc2)c(-c2cccc3c2oc2ccccc23)c1] | 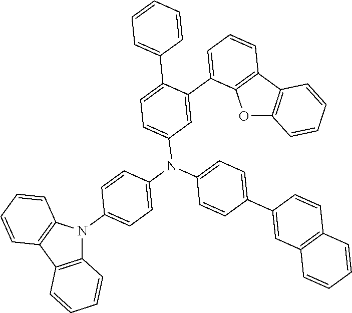 [SMILES: c1ccc(-c2ccc(N(c3ccc(-c4ccc5ccccc5c4)cc3)c3ccc(-n4c5ccccc5c5ccccc54)cc3)cc2-c2cccc3c2oc2ccccc23)cc1] | HPLC purity 98.65%, yield 68.5%; HRMS(EI): calculated value = 778.2984, measured value = 778.2982; |
| | Reactant A-2 | Reactant B-2 | Reactant C-2 | 128 | |
| Example 16 | 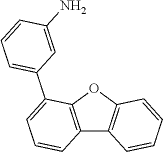 [SMILES: Nc1cccc(-c2cccc3c2oc2ccccc23)c1] | 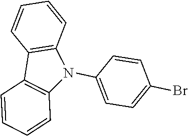 [SMILES: Brc1ccc(-n2c3ccccc3c3ccccc32)cc1] | 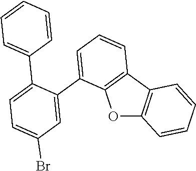 [SMILES: Brc1ccc(-c2ccccc2)c(-c2cccc3c2oc2ccccc23)c1] | 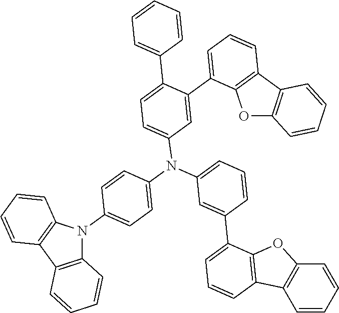 [SMILES: c1ccc(-c2ccc(N(c3ccc(-n4c5ccccc5c5ccccc54)cc3)c3cccc(-c4cccc5c4oc4ccccc45)c3)cc2-c2cccc3c2oc2ccccc23)cc1] | HPLC purity 97.25%, yield 65.0%; HRMS(EI): calculated value = 818.2933, measured value = 818.2922; |
| | Reactant A-3 | Reactant B-2 | Reactant C-2 | 130 | |

The reactants A, reactants B and reactants C in the above reactions were commercially available, or synthesized by a suzuki carbon-carbon coupling reaction or an Ullman carbon-nitrogen coupling reaction in one or more steps.

Take the synthesis of reactant C-3

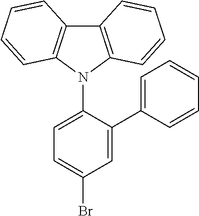 [SMILES: Brc1ccc(-n2c3ccccc3c3ccccc32)c(-c2ccccc2)c1]

as an example.

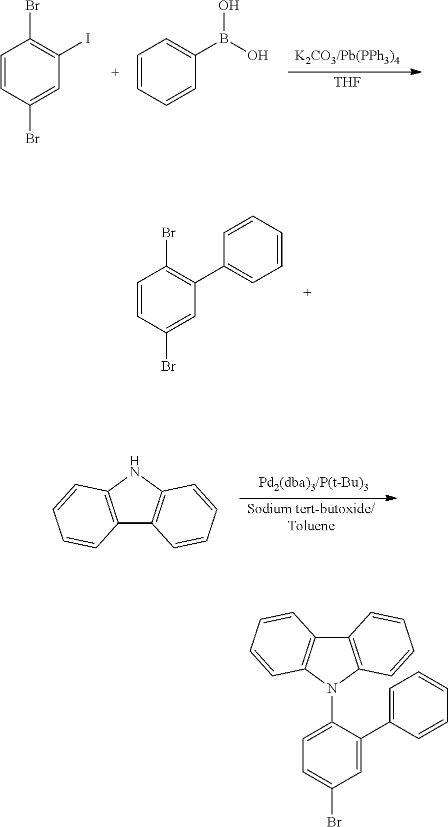 [SMILES: Brc1ccc(Br)c(I)c1 ; OB(O)c1ccccc1 ; Brc1ccc(Br)c(-c2ccccc2)c1 ; c1ccc2c(c1)[nH]c1ccccc12 ; "THF" ; Brc1ccc(-n2c3ccccc3c3ccccc32)c(-c2ccccc2)c1]

(1) Under the protection of nitrogen, 0.1 mol 2,5-dibromoiodobenzene, 0.1 mol phenylboronic acid, and 100 ml tetrahydrofuran (THF) were added into a 250 ml reaction flask. Then, 0.001 mol Pd(PPh₃)₄ and 20 ml of an aqueous 2M potassium carbonate solution were added to the reaction system. The reaction system was reacted at 70° C. to 90° C. for 10 to 24 h and cooled to room temperature naturally. The reaction solution was filtered. The filtrate was subjected to rotary evaporation under reduced pressure, and passed through a neutral silica gel column to obtain 2,5-dibromobiphenyl, with a HPLC purity of 99.25% and a yield of 98.0%.

(2) Under the protection of nitrogen, 0.01 mol of the intermediate product 2,5-dibromobiphenyl obtained in step (1), 0.01 mol carbazole and 150 ml of toluene were added to a 250 ml three-necked flask, stirred and mixed. Then, 5×10⁻⁵ mol Pd₂(dba)₃, 5×10⁻⁵ mol P(t-Bu)₃, and 0.03 mol sodium tert-butoxide were added, heated to 105° C., and reacted under reflux for 24 h. Samples were taken and applied onto a plate, which showed that the reaction was complete. The reaction mixture was naturally cooled to room temperature, and filtered. The filtrate was subjected to rotary evaporation until no more distillate was observed, and passed through a neutral silica gel column to obtain reactant C-3, with a HPLC purity of 99.65% and a yield of 82.5%. HRMS(EI): the molecular weight of the material=397.0466; the measured molecular weight of material=397.0449.

The compounds of the present invention were used as the hole transporting layer materials in light-emitting devices. The compounds of the present invention prepared in the above examples were tested in terms of thermal performance, T1 energy level, and HOMO energy level respectively. The test results are shown in Table 2.

TABLE 2

| Compounds | Tg (° C.) | Td (° C.) | T1 (eV) | HOMO (ev) |
|---|---|---|---|---|
| Compound 1 | 143 | 407 | 2.64 | 5.66 |
| Compound 6 | 140 | 410 | 2.62 | 5.55 |
| Compound 11 | 139 | 404 | 2.6 | 5.52 |
| Compound 21 | 142 | 411 | 2.70 | 5.70 |
| Compound 26 | 135 | 404 | 2.73 | 5.72 |
| Compound 42 | 139 | 410 | 2.59 | 5.56 |
| Compound 47 | 136 | 410 | 2.60 | 5.65 |
| Compound 54 | 136 | 405 | 2.60 | 5.50 |
| Compound 62 | 140 | 411 | 2.68 | 5.65 |
| Compound 68 | 139 | 412 | 2.70 | 5.69 |
| Compound 78 | 138 | 412 | 2.68 | 5.69 |
| Compound 90 | 141 | 411 | 2.75 | 5.73 |
| Compound 107 | 138 | 408 | 2.59 | 5.54 |
| Compound 117 | 139 | 411 | 2.63 | 5.67 |
| Compound 128 | 142 | 408 | 5.58 | 5.56 |
| Compound 130 | 140 | 410 | 2.65 | 5.65 |
| Compound 154 | 146 | 411 | 2.64 | 5.61 |
| Compound 163 | 153 | 418 | 2.67 | 5.69 |
| Compound 173 | 148 | 414 | 2.61 | 5.63 |
| Compound 177 | 157 | 419 | 2.63 | 5.52 |
| Compound 181 | 153 | 422 | 2.58 | 5.49 |

Note: The triplet energy level T1 was tested using a F4600 fluorescence spectrometer from Hitachi, with the materials being tested in a 2*10⁻⁵ toluene solution; the glass transition temperature Tg was measured by differential scanning calorimetry (DSC) using a DSC204F1 differential scanning calorimeter from Netzsch, Germany, with a heating rate of 10° C./min; the thermal weight loss temperature Td, which is the temperature for 1% weight loss in a nitrogen atmosphere, was measured using a TGA-50H thermogravimetric analyzer from Shimadzu Corporation, Japan, with the flow rate of nitrogen being 20 mL/min; and the highest occupied molecular orbital HOMO energy level was tested using an ionization energy measuring system (IPS3) in the atmospheric environment.

As seen from the above data in Table 2, the organic compounds of the present invention have appropriate HOMO energy levels and can be used in hole transporting layers. The organic compounds with core structures of triarylamine of the present invention have relatively high triplet energy levels and relatively high thermal stability, such that the manufactured OLEDs containing the organic compound of the invention have improved efficiency and prolonged service lives.

In the following, the effects of using the compounds of the present invention as hole transporting layer materials in the devices are detailed through Device Examples 1-16 and Device Comparative Example 1. Device Examples 2-16 and Device Comparative 1 were performed by exactly repeating the preparation process of Device Example 1, including the same substrate materials and electrode materials, and also the same thickness of the electrode material film, except that the hole transporting layer materials and electron blocking layer materials were changed. The laminated structures of the devices are shown in Table 3. The test results of the performance of each device are shown in Tables 4 and 5.

### Device Example 1

ITO was used as the anode, Al as the cathode, GH-1, GH-2 and GD-1 mixed in a weight ratio of 45:45:10 as the light-emitting layer material, HAT-CN as the hole injection layer material, compound 6 prepared in the example of the present invention as the hole transporting layer material, EB-1 as the electron blocking layer material, ET-1 and Liq as the electron transporting layer material, and LiF as the electron injection layer material. The specific manufacturing steps are as follows:

a) an ITO anode layer 2 on a transparent substrate layer 1 was ultrasonically cleaned with deionized water, acetone, and ethanol for 15 minutes in each case, and then treated in a plasma cleaner for 2 minutes;

b) on the ITO anode layer 2, the hole injection layer material HAT-CN was deposited by vacuum evaporation, and this layer with a thickness of 10 nm was used as a hole injection layer 3;

c) on the hole injection layer 3, the hole transporting layer material compound 6 was deposited by vacuum evaporation, and this layer with a thickness of 60 nm was a hole injection layer 4;

c) on the first hole injection layer 4, the electron blocking layer material EB-1 was deposited by vacuum evaporation, and this layer with a thickness of 20 nm was an electron blocking layer 5;

e) on the electron blocking layer 5, a light-emitting layer 6 was deposited by vacuum evaporation, wherein the light-emitting layer 6 has a thickness of 30 nm, and in the light-emitting layer 6, the host materials were GH-1 and GH-2, and the doping materials were GD-1, with GH-1, GH-2 and GD-1 present in a mass ratio of 45:45:10;

f) on the light-emitting layer 6, the electron transporting materials ET-1 and Liq were deposited by vacuum evaporation in a mass ratio of 1:1, and this layer of organic materials with a thickness of 40 nm was used as a hole blocking/electron transporting layer 7;

g) on the hole blocking/electron transporting layer 7, the electron injection material LiF was deposited by vacuum evaporation, and this layer with a thickness of 1 nm was an electron injection layer 8; and

h) on the electron injection layer 8, the cathode Al (100 nm) was deposited by vacuum evaporation, and this layer was a cathode reflective electrode layer 9.

After the light-emitting devices were prepared according to the above steps, the efficiency data and the luminescence decay lifetime of the devices were measured. The results are shown in Table 4. The molecular structures of relevant materials are as follows:

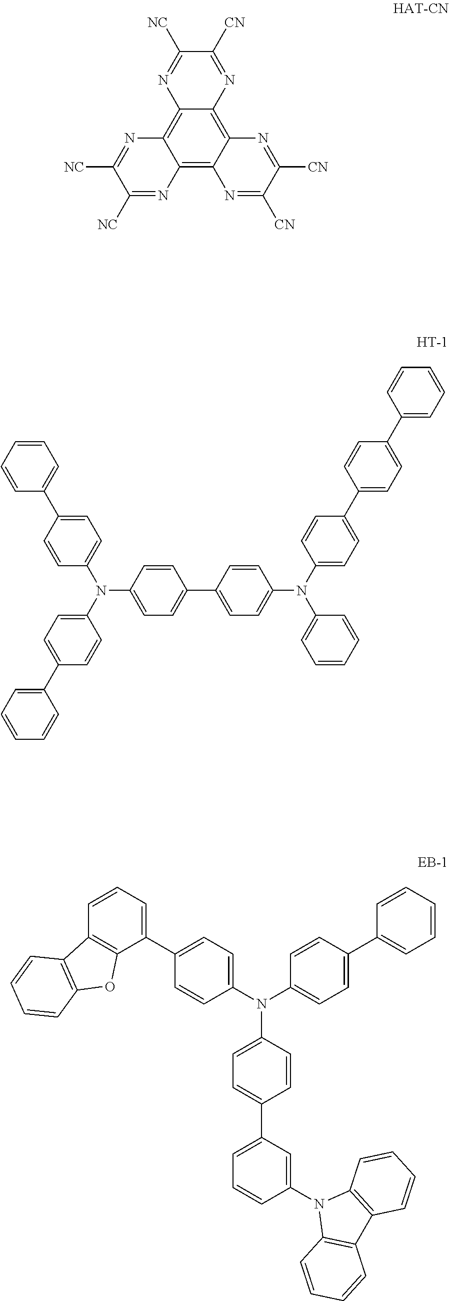 [SMILES: N#Cc1nc2c3nc(C#N)c(C#N)nc3c3nc(C#N)c(C#N)nc3c2nc1C#N ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(-c2ccc(N(c3ccc(-c4cccc(-n5c6ccccc6c6ccccc65)c4)cc3)c3ccc(-c4cccc5c4oc4ccccc45)cc3)cc2)cc1]

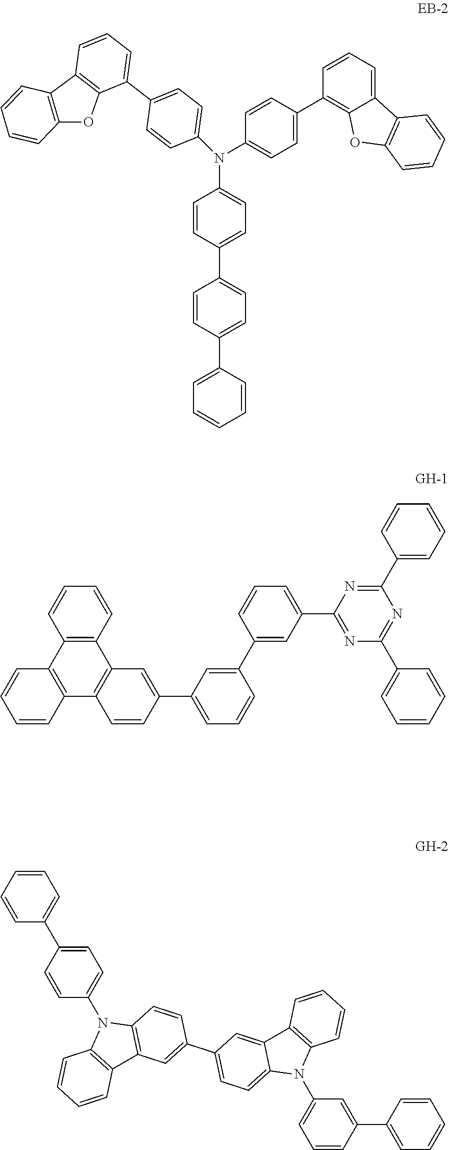 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2nc(-c3ccccc3)nc(-c3cccc(-c4cccc(-c5ccc6c7ccccc7c7ccccc7c6c5)c4)c3)n2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

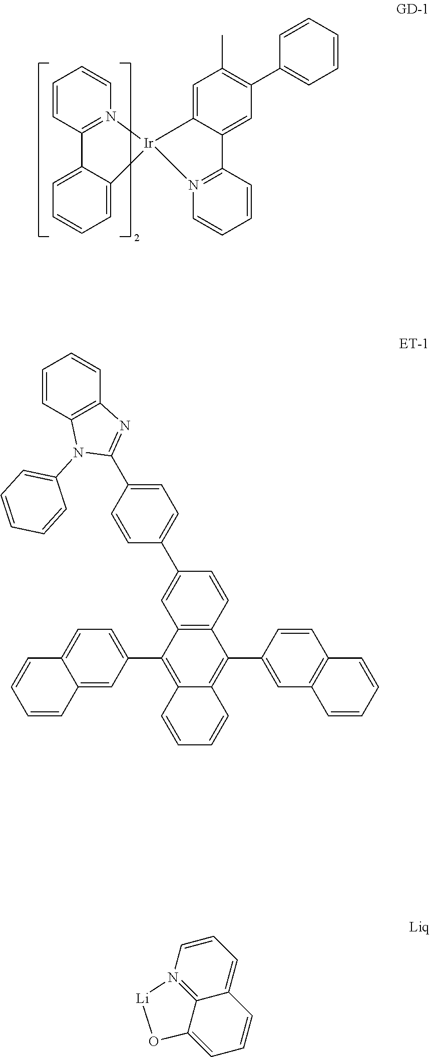

TABLE 3

| | | | | | Hole blocking/electron | |
|---|---|---|---|---|---|---|
| Device | Hole injection | Hole transporting | Electron blocking | Light-emitting layer/ | transporting layer | Electron injection |
| Examples | layer (thickness) | layer (thickness) | layer (thickness) | mass ratio (thickness) | (thickness) | layer (thickness) |
| 1 | HAT-CN | Compound 6 | Compound EB-1 | GH-1:GH-2:GD-1 | ET-1:Liq = 1:1 | LiF (1 nm) |
| | (10 nm) | (60 nm) | (20 nm) | 45:45:10 (30 nm) | (40 nm) | |
| 2 | HAT-CN | Compound 11 | Compound EB-1 | GH-1:GH-2:GD-1 | ET-1:Liq = 1:1 | LiF (1 nm) |
| | (10 nm) | (60 nm) | (20 nm) | 45:45:10 (30 nm) | (40 nm) | |
| 3 | HAT-CN | Compound 42 | Compound EB-1 | GH-1:GH-2:GD-1 | ET-1:Liq = 1:1 | LiF (1 nm) |
| | (10 nm) | (60 nm) | (20 nm) | 45:45:10 (30 nm) | (40 nm) | |
| 4 | HAT-CN | Compound 54 | Compound EB-1 | GH-1:GH-2:GD-1 | ET-1:Liq = 1:1 | LiF (1 nm) |
| | (10 nm) | (60 nm) | (20 nm) | 45:45:10 (30 nm) | (40 nm) | |
| 5 | HAT-CN | Compound 107 | Compound EB-1 | GH-1:GH-2:GD-1 | ET-1:Liq = 1:1 | LiF (1 nm) |
| | (10 nm) | (60 nm) | (20 nm) | 45:45:10 (30 nm) | (40 nm) | |
| 6 | HAT-CN | Compound 128 | Compound EB-1 | GH-1:GH-2:GD-1 | ET-1:Liq = 1:1 | LiF (1 nm) |
| | (10 nm) | (60 nm) | (20 nm) | 45:45:10 (30 nm) | (40 nm) | |
| 7 | HAT-CN | Compound HT-1 | Compound 1 | GH-1:GH-2:GD-1 | ET-1:Liq = 1:1 | LiF (1 nm) |
| | (10 nm) | (60 nm) | (20 nm) | 45:45:10 (30 nm) | (40 nm) | |
| 8 | HAT-CN | Compound HT-1 | Compound 47 | GH-1:GH-2:GD-1 | ET-1:Liq = 1:1 | LiF (1 nm) |
| | (10 nm) | (60 nm) | (20 nm) | 45:45:10 (30 nm) | (40 nm) | |
| 9 | HAT-CN | Compound HT-1 | Compound 62 | GH-1:GH-2:GD-1 | ET-1:Liq = 1:1 | LiF (1 nm) |
| | (10 nm) | (60 nm) | (20 nm) | 45:45:10 (30 nm) | (40 nm) | |
| 10 | HAT-CN | Compound HT-1 | Compound 130 | GH-1:GH-2:GD-1 | ET-1:Liq = 1:1 | LiF (1 nm) |
| | (10 nm) | (60 nm) | (20 nm) | 45:45:10 (30 nm) | (40 nm) | |
| 11 | HAT-CN | Compound HT-1 | Compound 21:EB-2 = | GH-1:GH-2:GD-1 | ET-1:Liq = 1:1 | LiF (1 nm) |
| | (10 nm) | (60 nm) | 1:1(20 nm) | 45:45:10 (30 nm) | (40 nm) | |
| 12 | HAT-CN | Compound HT-1 | Compound 26:EB-2 = | GH-1:GH-2:GD-1 | ET-1:Liq = 1:1 | LiF (1 nm) |
| | (10 nm) | (60 nm) | 1:1(20 nm) | 45:45:10 (30 nm) | (40 nm) | |
| 13 | HAT-CN | Compound HT-1 | Compound 68:EB-2 = | GH-1:GH-2:GD-1 | ET-1:Liq = 1:1 | LiF (1 nm) |
| | (10 nm) | (60 nm) | 1:1(20 nm) | 45:45:10 (30 nm) | (40 nm) | |
| 14 | HAT-CN | Compound HT-1 | Compound 78: | GH-1:GH-2:GD-1 | ET-1:Liq = 1:1 | LiF (1 nm) |
| | (10 nm) | (60 nm) | EB-2 = | 45:45:10 (30 nm) | (40 nm) | |
| | | | 1:1(20 nm) | | | |
| 15 | HAT-CN | Compound HT-1 | Compound 90: | GH-1:GH-2:GD-1 | ET-1:Liq = 1:1 | LiF (1 nm) |
| | (10 nm) | (60 nm) | EB-2 = | 45:45:10 (30 nm) | (40 nm) | |
| | | | 1:1(20 nm) | | | |
| 16 | HAT-CN | Compound HT-1 | Compound 117: | GH-1:GH-2:GD-1 | ET-1:Liq = 1:1 | LiF (1 nm) |
| | (10 nm) | (60 nm) | EB-2 = 1:1(20 nm) | 45:45:10 (30 nm) | (40 nm) | |
| 17 | HAT-CN | Compound HT-1 | Compound 154 | GH-1:GH-2:GD-1 | ET-1:Liq = 1:1 | LiF (1 nm) |
| | (10 nm) | (60 nm) | (20 nm) | 45:45:10 (30 nm) | (40 nm) | |
| 18 | HAT-CN | Compound HT-1 | Compound 163 | GH-1:GH-2:GD-1 | ET-1:Liq = 1:1 | LiF (1 nm) |
| | (10 nm) | (60 nm) | (20 nm) | 45:45:10 (30 nm) | (40 nm) | |
| 19 | HAT-CN | Compound HT-1 | Compound 173 | GH-1:GH-2:GD-1 | ET-1:Liq = 1:1 | LiF (1 nm) |
| | (10 nm) | (60 nm) | (20 nm) | 45:45:10 (30 nm) | (40 nm) | |
| 20 | HAT-CN | Compound 177 | Compound EB-1 | GH-1:GH-2:GD-1 | ET-1:Liq = 1:1 | LiF (1 nm) |
| | (10 nm) | (60 nm) | (20 nm) | 45:45:10 (30 nm) | (40 nm) | |
| 21 | HAT-CN | Compound 181 | Compound EB-1 | GH-1:GH-2:GD-1 | ET-1:Liq = 1:1 | LiF (1 nm) |
| | (10 nm) | (60 nm) | (20 nm) | 45:45:10 (30 nm) | (40 nm) | |
| Comparative | HAT-CN | Compound HT-1 | Compound EB-1 | GH-1:GH-2:GD-1 | ET-1:Liq = 1:1 | LiF (1 nm) |
| Example 1 | (10 nm) | (60 nm) | (20 nm) | 45:45:10 (30 nm) | (40 nm) | |

TABLE 4

| | @10 mA/cm² | | | |
|---|---|---|---|---|
| | Current | | Lifetime | Driving |
| | efficiency | | LT95(Hr)@5000 | voltage |
| No. | (cd/A) | Color | nits | (V) |
| Device Example1 | 65.2 | Green light | 54.1 | 3.80 |
| Device Example2 | 68.7 | Green light | 53.5 | 3.99 |
| Device Example3 | 65.9 | Green light | 53.6 | 4.02 |
| Device Example4 | 67.2 | Green light | 53.7 | 3.87 |
| Device Example5 | 65.7 | Green light | 52.6 | 3.88 |
| Device Example6 | 66.2 | Green light | 52.3 | 3.77 |
| Device Example7 | 68.7 | Green light | 55.6 | 3.74 |
| Device Example8 | 67.3 | Green light | 53.7 | 3.67 |
| Device Example9 | 66.9 | Green light | 54.6 | 3.67 |
| Device Example10 | 69.2 | Green light | 55.7 | 3.71 |
| Device Example11 | 72.2 | Green light | 66.2 | 3.82 |
| Device Example12 | 69.7 | Green light | 67.5 | 3.64 |
| Device Example13 | 70.8 | Green light | 68.2 | 3.81 |
| Device Example14 | 68.1 | Green light | 66.6 | 3.76 |
| Device Example15 | 73.2 | Green light | 67.8 | 3.66 |
| Device Example16 | 69.6 | Green light | 65.5 | 3.79 |
| Device Example17 | 70.1 | Green light | 54.8 | 3.85 |
| Device Example18 | 74.7 | Green light | 51.0 | 3.91 |
| Device Example19 | 68.5 | Green light | 53.7 | 4.03 |
| Device Example20 | 69.2 | Green light | 61.7 | 3.67 |
| Device Example21 | 66.5 | Green light | 65.1 | 3.74 |
| Comparative | 55 | Green light | 30.5 | 4.4 |
| Example 1 | | | | |

As can be seen from the data in Table 4, the organic light-emitting devices of the present invention have been greatly improved in terms of both the efficiency and lifetime as compared with OLEDs of known materials.

In order to compare the efficiency decay of different devices at high current density, an efficiency decay coefficient φ was defined. This coefficient represents the ratio of the difference between the maximum efficiency μₘₐₓ of the device and the minimum efficiency μₘᵢₙ, of the device to the maximum efficiency at a driving current of 100 mA/cm². The larger the φ value is, the more serious the efficiency roll-off of the device is, whereas a smaller φ value shows that rapid decay of the device at a high current density has been controlled. The efficiency decay coefficient φ was measured for Device Examples 1-16 and Device Comparative Example 1 respectively. The results are shown in Table 5.

TABLE 5

| | Efficiency |
|---|---|
| | decay |
| Device no. | coefficient φ |
| Device Example 1 | 0.22 |
| Device Example 2 | 0.22 |
| Device Example 3 | 0.25 |
| Device Example 4 | 0.19 |
| Device Example 5 | 0.21 |
| Device Example 6 | 0.22 |
| Device Example 7 | 0.19 |
| Device Example 8 | 0.24 |
| Device Example 9 | 0.22 |
| Device Example 10 | 0.22 |
| Device Example 11 | 0.20 |
| Device Example 12 | 0.20 |
| Device Example 13 | 0.23 |
| Device Example 14 | 0.20 |
| Device Example 15 | 0.21 |
| Device Example 16 | 0.21 |
| Device Example 17 | 0.23 |
| Device Example 18 | 0.24 |
| Device Example 19 | 0.23 |
| Device Example 20 | 0.18 |
| Device Example 21 | 0.19 |
| Comparative Example 1 | 0.35 |

As seen from the data in Table 5, by comparing the efficiency decay coefficients for the examples and the comparative example, it can be seen that the efficiency roll-off of the organic light-emitting devices of the present invention are effectively reduced.

Furthermore, the OLEDs prepared from the materials of the present invention have relatively stable efficiency when working at low temperature. The efficiency of Device Examples 1, 7, 11 and Device Comparative Example 1 was tested at a temperature ranging from −10° C. to 80° C. The results are shown in Table 6 and FIG. 2.

TABLE 6

| Current | Temperature (° C.) | | | | | | | | | |
|---|---|---|---|---|---|---|---|---|---|---|
| efficiency (cd/A) | −10 | 0 | 10 | 20 | 30 | 40 | 50 | 60 | 70 | 80 |
| Device Example 1 | 61.2 | 63.2 | 64.8 | 65.2 | 65.3 | 66.2 | 65.6 | 66.2 | 65.4 | 65.1 |
| Device Example 7 | 67.2 | 67.9 | 68.5 | 68.7 | 68.7 | 68.8 | 69.5 | 69.3 | 69.1 | 68.9 |
| Device Example 11 | 71.2 | 71.5 | 72 | 72.2 | 72.2 | 72.5 | 73.1 | 73 | 72.9 | 72.5 |
| Device Comparative | 49.5 | 49.8 | 54.1 | 55 | 55.8 | 57 | 57.8 | 55.3 | 55.1 | 49.2 |
| Example 1 | | | | | | | | | | |

As can be seen from the data in Table 6 and FIG. 2, compared with Device Comparative Example 1, Device Examples 1, 7 and 11, which have device structures obtained by combining the materials of the present invention with the known materials, not only have high efficiencies at low temperatures, but also show a steady increase in efficiency during temperature rising.

What are described above are merely preferred embodiments of the present invention, and are not to limit the present invention, and any modification, equivalent and improvement made within the spirit and principles of the present invention shall be covered in the protection scope of the present invention.

1. A compound with a core structure of triarylamine, characterized in that the compound has a structure represented by general formula (1): 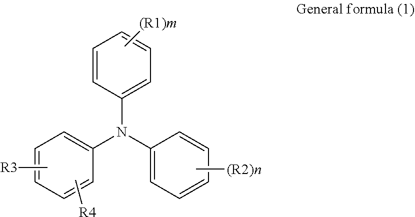
wherein
m and n each independently represents an integer of 1, 2 or 3;
R₁, R₂, R₃ and R₄ each independently represents substituted or unsubstituted phenyl, substituted or unsubstituted biphenyl, substituted or unsubstituted naphthyl, or a structure represented by general formula (2) or general formula (3), with R₃ and R₄ positioned ortho to each other; 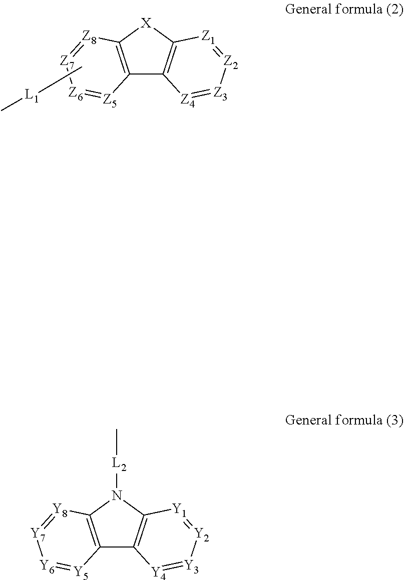
in the general formula (2) and the general formula (3), L₁ and L₂ each independently represents a single bond, substituted or unsubstituted phenylene, substituted or unsubstituted naphthylene, or substituted or unsubstituted biphenylene;
in the general formula (2), X represents —O—, —S—, —C(R₅)(R₆)— or —N(R₇)—;
Z₁-Z₈ each independently represents CH or N, with at most 4 N;
in the general formula (2), Z₅, Z₆, Z₇ or Z₈ to which L₁ is bonded represents a carbon atom;
in the general formula (3), Y₁-Y₈ each independently represents CH or N, with at most 4 N;
R₅ to R₇ each independently represents one of C₁₋₂₀ alkyl, C₆₋₃₀ aryl, and substituted or unsubstituted 5- to 30-membered heteroaryl containing one or more heteroatoms, wherein R₅ and R₆ together with the atom to which they are bonded may form a 5-membered to 30-membered alicyclic or aromatic ring;
the substituent is halogen, cyano, C₁₋₁₀ alkyl or C₆₋₂₀ aryl.

2. The compound according to claim 1, characterized in that R₅ to R₇ each independently represents methyl, ethyl, propyl, isopropyl, butyl, tert-butyl, pentyl, phenyl, biphenyl, terphenyl, naphthyl, pyridyl or furyl.

3. The compound according to claim 1, characterized in that the structure of the general formula (2) is represented by any one of

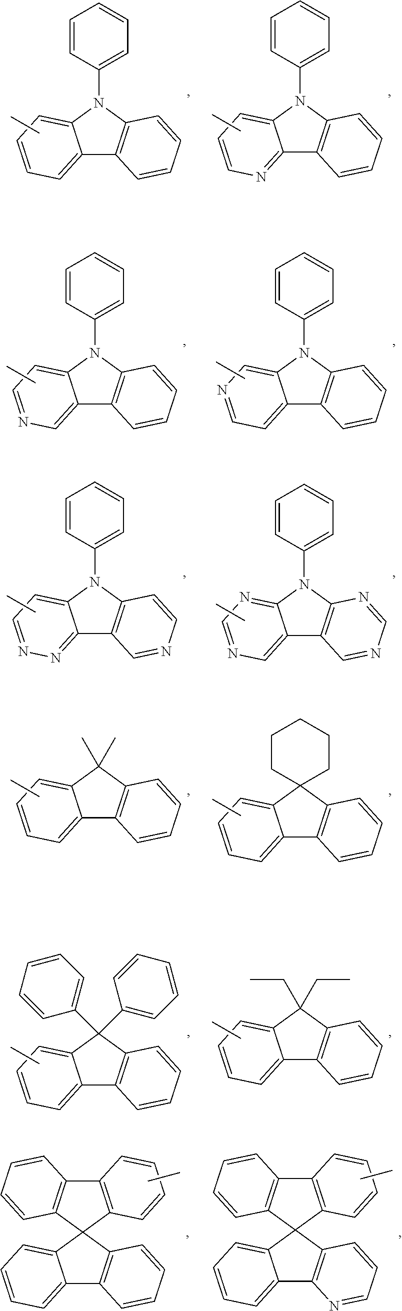

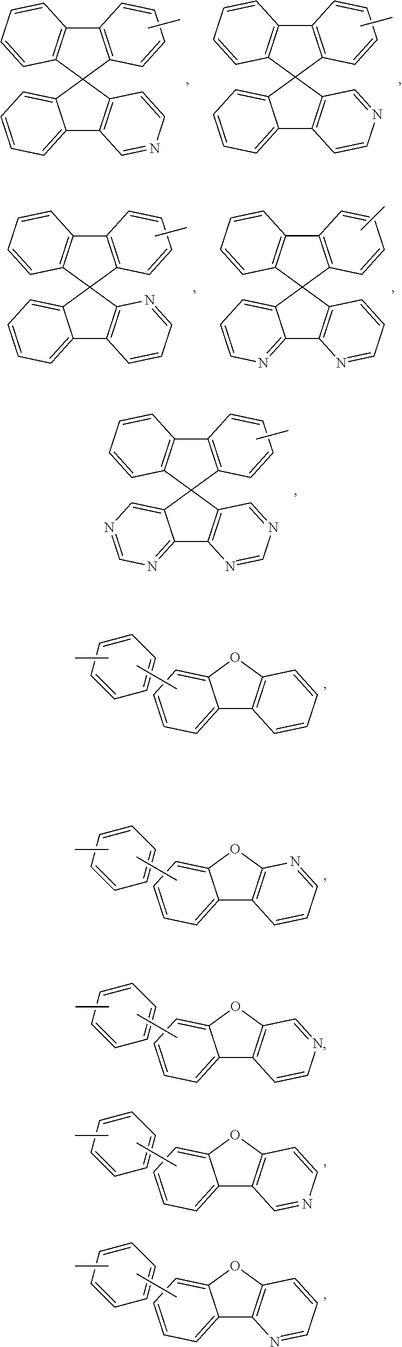

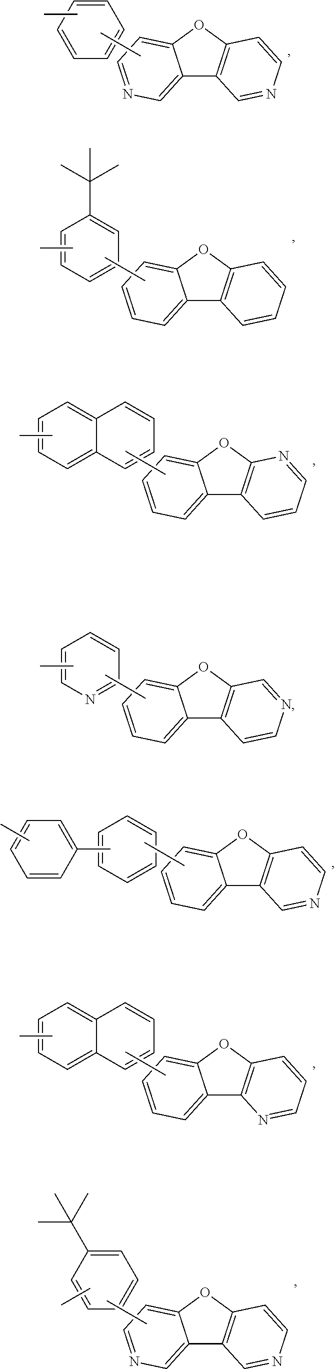

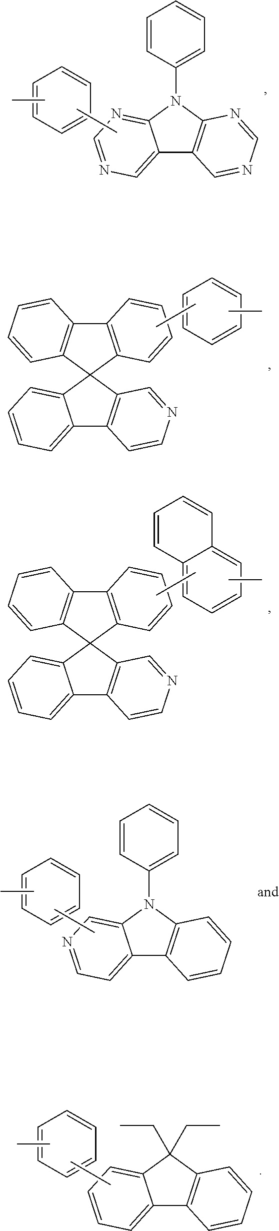

4. The compound according to claim 1, characterized in that the structure of the general formula (3) is represented by any one of

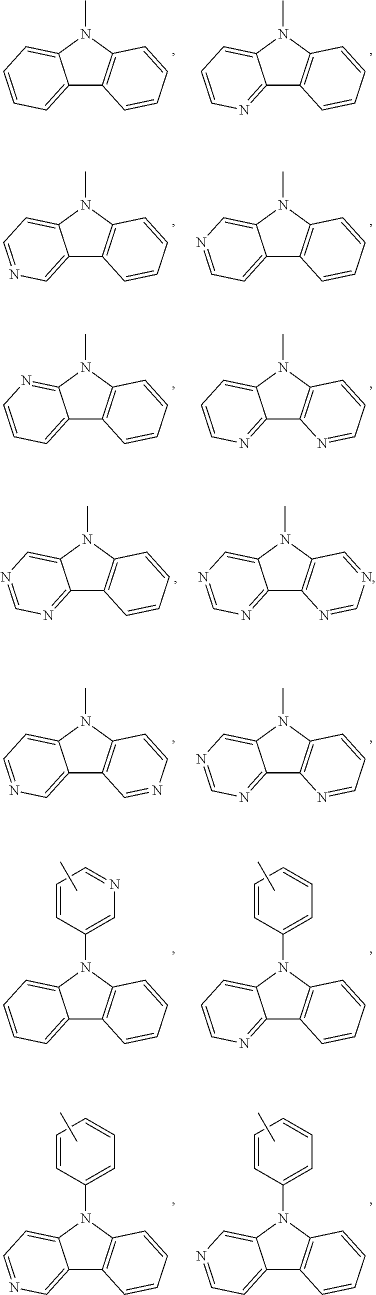

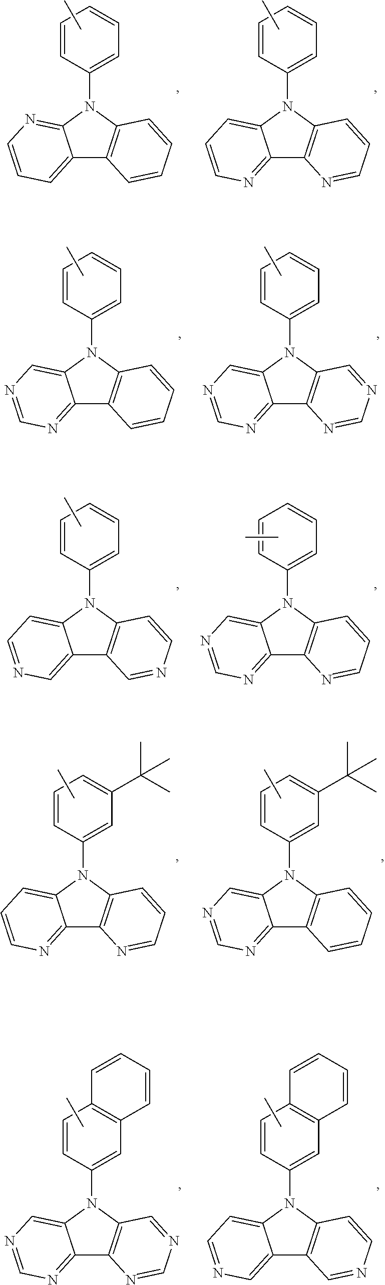

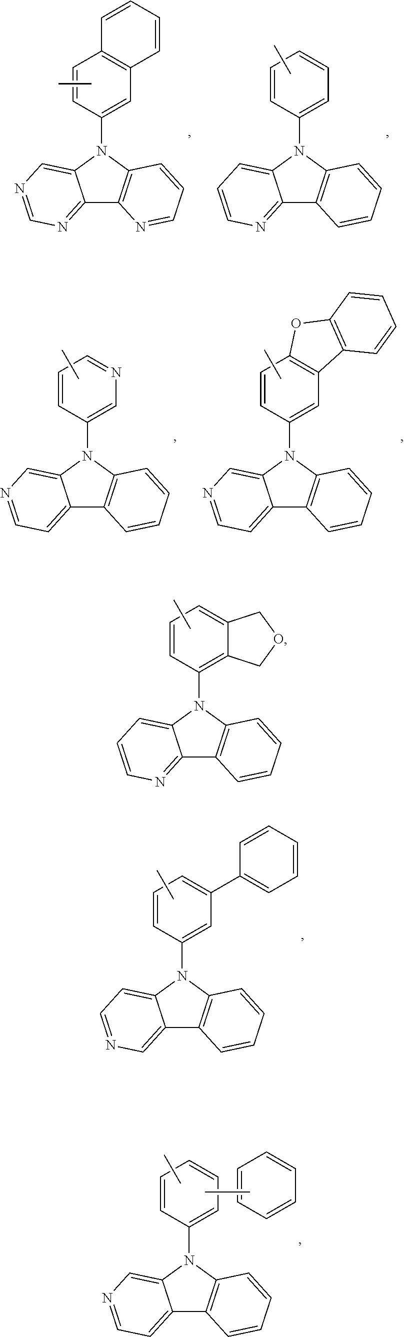

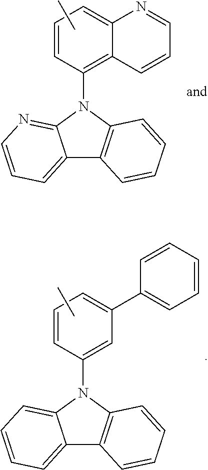

5. The compound according to claim 1, characterized in that the compound has a specific structure of any one of

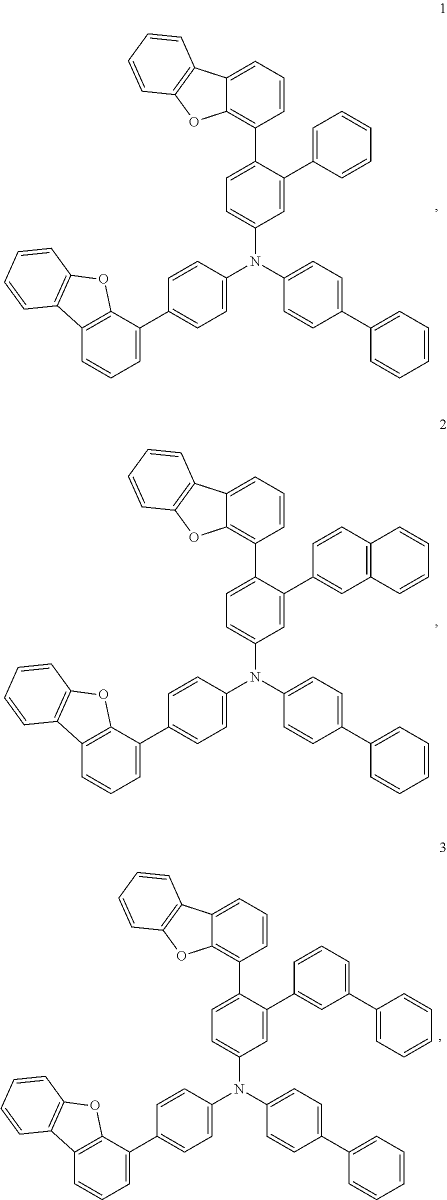 [SMILES: c1ccc(-c2ccc(N(c3ccc(-c4cccc5c4oc4ccccc45)cc3)c3ccc(-c4cccc5c4oc4ccccc45)c(-c4ccccc4)c3)cc2)cc1 ; c1ccc(-c2ccc(N(c3ccc(-c4cccc5c4oc4ccccc45)cc3)c3ccc(-c4cccc5c4oc4ccccc45)c(-c4ccc5ccccc5c4)c3)cc2)cc1 ; c1ccc(-c2ccc(N(c3ccc(-c4cccc5c4oc4ccccc45)cc3)c3ccc(-c4cccc5c4oc4ccccc45)c(-c4cccc(-c5ccccc5)c4)c3)cc2)cc1]

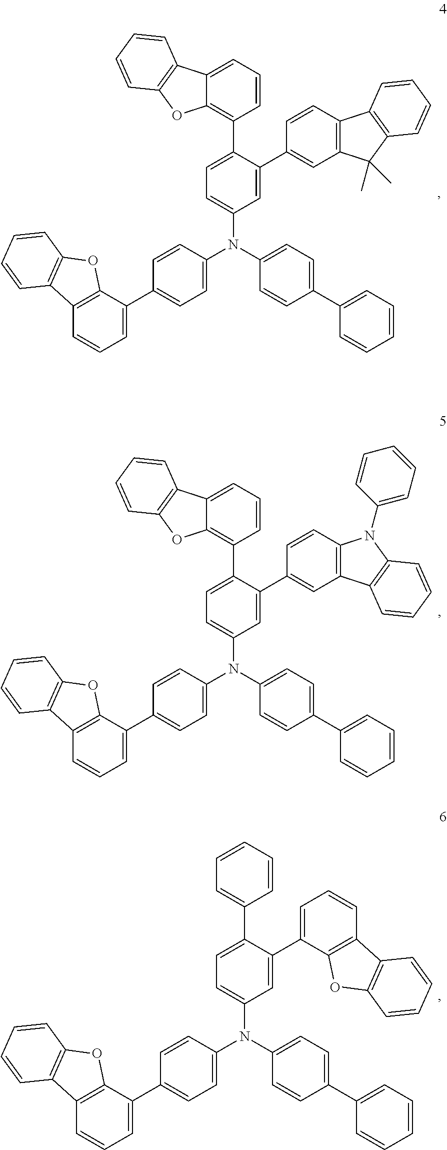 [SMILES: CC1(C)c2ccccc2-c2ccc(-c3cc(N(c4ccc(-c5ccccc5)cc4)c4ccc(-c5cccc6c5oc5ccccc56)cc4)ccc3-c3cccc4c3oc3ccccc34)cc21 ; c1ccc(-c2ccc(N(c3ccc(-c4cccc5c4oc4ccccc45)cc3)c3ccc(-c4cccc5c4oc4ccccc45)c(-c4ccc5c(c4)c4ccccc4n5-c4ccccc4)c3)cc2)cc1 ; c1ccc(-c2ccc(N(c3ccc(-c4cccc5c4oc4ccccc45)cc3)c3ccc(-c4ccccc4)c(-c4cccc5c4oc4ccccc45)c3)cc2)cc1]

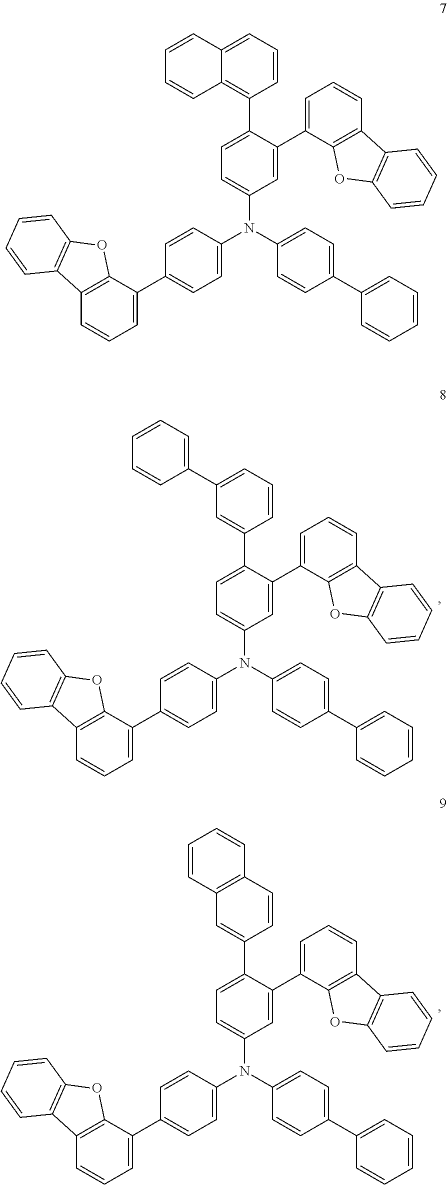 [SMILES: c1ccc(-c2ccc(N(c3ccc(-c4cccc5c4oc4ccccc45)cc3)c3ccc(-c4cccc5ccccc45)c(-c4cccc5c4oc4ccccc45)c3)cc2)cc1 ; c1ccc(-c2ccc(N(c3ccc(-c4cccc5c4oc4ccccc45)cc3)c3ccc(-c4cccc(-c5ccccc5)c4)c(-c4cccc5c4oc4ccccc45)c3)cc2)cc1 ; c1ccc(-c2ccc(N(c3ccc(-c4cccc5c4oc4ccccc45)cc3)c3ccc(-c4ccc5ccccc5c4)c(-c4cccc5c4oc4ccccc45)c3)cc2)cc1]

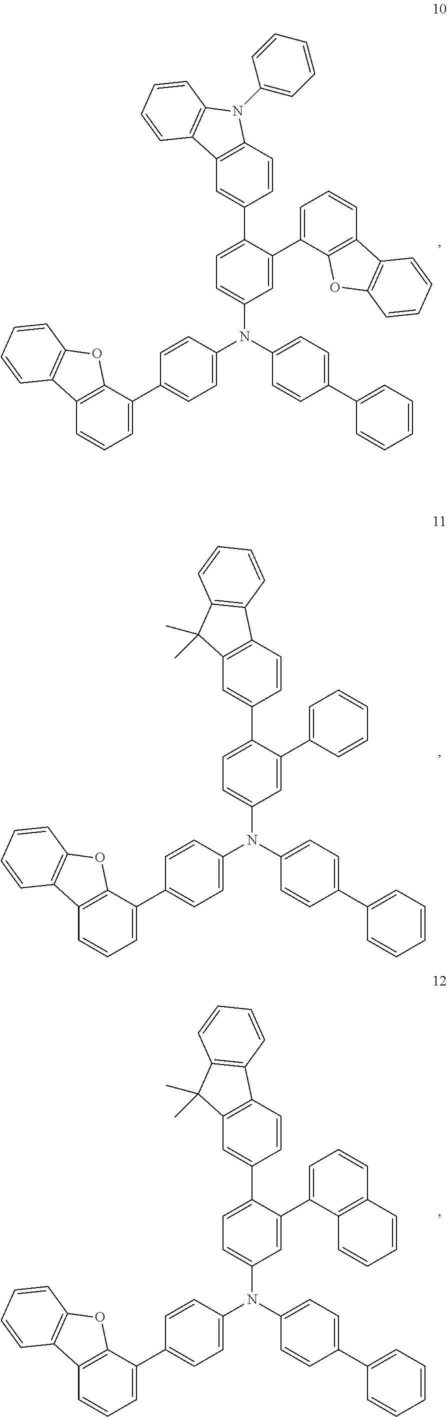 [SMILES: c1ccc(-c2ccc(N(c3ccc(-c4cccc5c4oc4ccccc45)cc3)c3ccc(-c4ccc5c(c4)c4ccccc4n5-c4ccccc4)c(-c4cccc5c4oc4ccccc45)c3)cc2)cc1 ; CC1(C)c2ccccc2-c2ccc(-c3ccc(N(c4ccc(-c5ccccc5)cc4)c4ccc(-c5cccc6c5oc5ccccc56)cc4)cc3-c3ccccc3)cc21 ; CC1(C)c2ccccc2-c2ccc(-c3ccc(N(c4ccc(-c5ccccc5)cc4)c4ccc(-c5cccc6c5oc5ccccc56)cc4)cc3-c3cccc4ccccc34)cc21]

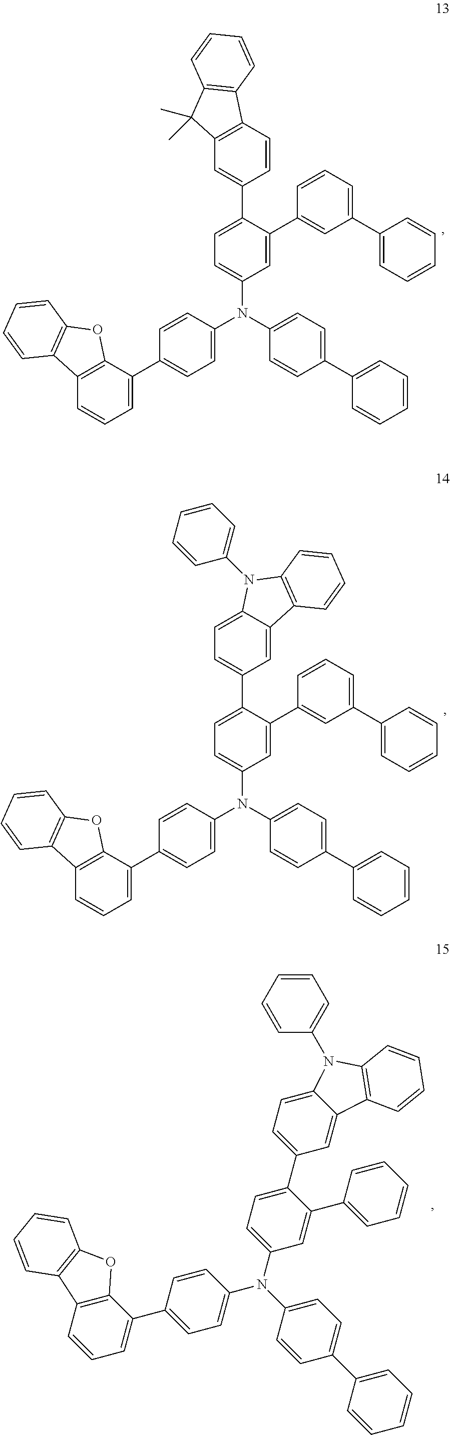 [SMILES: CC1(C)c2ccccc2-c2ccc(-c3ccc(N(c4ccc(-c5ccccc5)cc4)c4ccc(-c5cccc6c5oc5ccccc56)cc4)cc3-c3cccc(-c4ccccc4)c3)cc21 ; c1ccc(-c2ccc(N(c3ccc(-c4cccc5c4oc4ccccc45)cc3)c3ccc(-c4ccc5c(c4)c4ccccc4n5-c4ccccc4)c(-c4cccc(-c5ccccc5)c4)c3)cc2)cc1 ; c1ccc(-c2ccc(N(c3ccc(-c4cccc5c4oc4ccccc45)cc3)c3ccc(-c4ccc5c(c4)c4ccccc4n5-c4ccccc4)c(-c4ccccc4)c3)cc2)cc1]

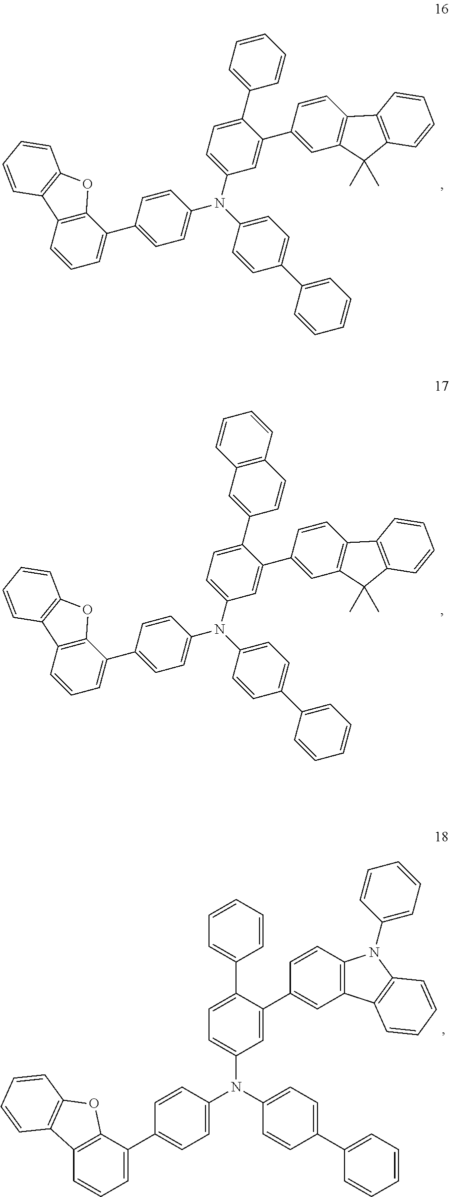 [SMILES: CC1(C)c2ccccc2-c2ccc(-c3cc(N(c4ccc(-c5ccccc5)cc4)c4ccc(-c5cccc6c5oc5ccccc56)cc4)ccc3-c3ccccc3)cc21 ; CC1(C)c2ccccc2-c2ccc(-c3cc(N(c4ccc(-c5ccccc5)cc4)c4ccc(-c5cccc6c5oc5ccccc56)cc4)ccc3-c3ccc4ccccc4c3)cc21 ; c1ccc(-c2ccc(N(c3ccc(-c4cccc5c4oc4ccccc45)cc3)c3ccc(-c4ccccc4)c(-c4ccc5c(c4)c4ccccc4n5-c4ccccc4)c3)cc2)cc1]

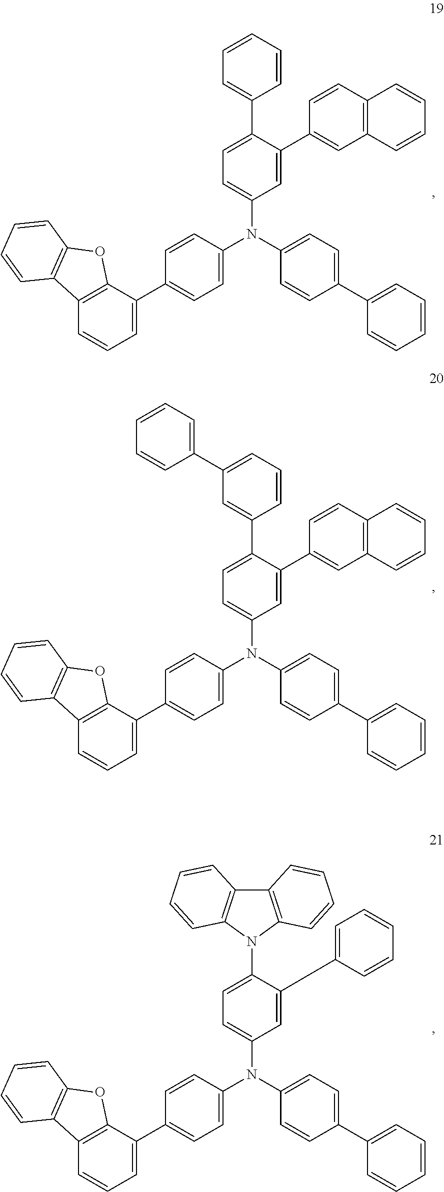 [SMILES: c1ccc(-c2ccc(N(c3ccc(-c4cccc5c4oc4ccccc45)cc3)c3ccc(-c4ccccc4)c(-c4ccc5ccccc5c4)c3)cc2)cc1 ; c1ccc(-c2ccc(N(c3ccc(-c4cccc5c4oc4ccccc45)cc3)c3ccc(-c4cccc(-c5ccccc5)c4)c(-c4ccc5ccccc5c4)c3)cc2)cc1 ; c1ccc(-c2ccc(N(c3ccc(-c4cccc5c4oc4ccccc45)cc3)c3ccc(-n4c5ccccc5c5ccccc54)c(-c4ccccc4)c3)cc2)cc1]

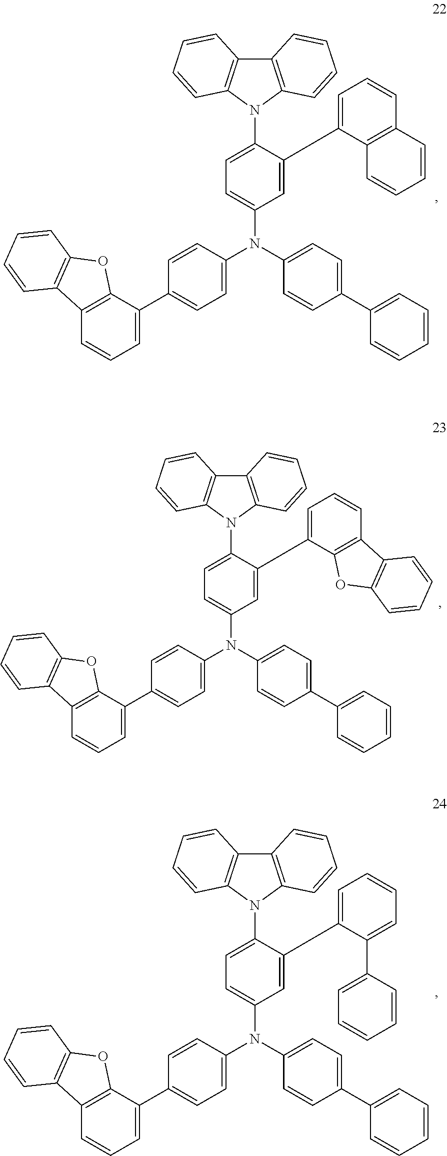 [SMILES: c1ccc(-c2ccc(N(c3ccc(-c4cccc5c4oc4ccccc45)cc3)c3ccc(-n4c5ccccc5c5ccccc54)c(-c4cccc5ccccc45)c3)cc2)cc1 ; c1ccc(-c2ccc(N(c3ccc(-c4cccc5c4oc4ccccc45)cc3)c3ccc(-n4c5ccccc5c5ccccc54)c(-c4cccc5c4oc4ccccc45)c3)cc2)cc1 ; c1ccc(-c2ccc(N(c3ccc(-c4cccc5c4oc4ccccc45)cc3)c3ccc(-n4c5ccccc5c5ccccc54)c(-c4ccccc4-c4ccccc4)c3)cc2)cc1]

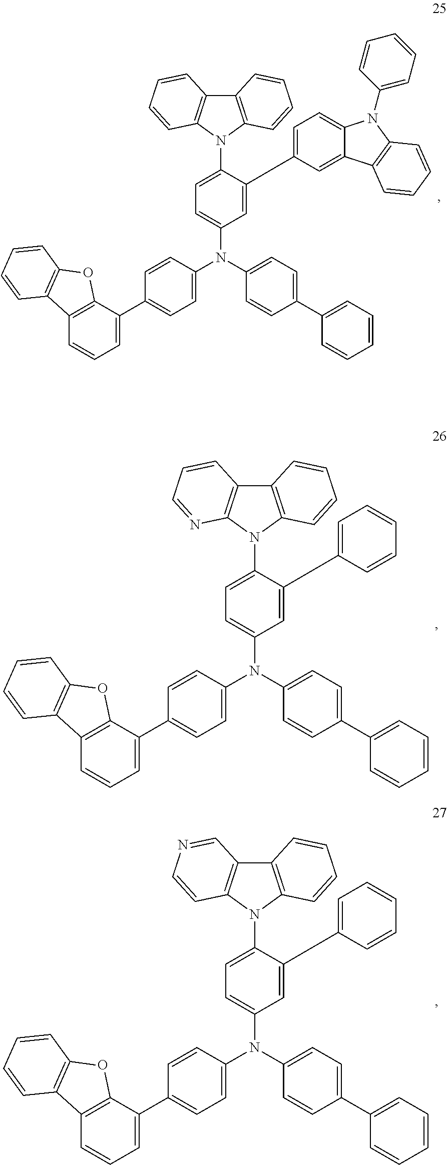 [SMILES: c1ccc(-c2ccc(N(c3ccc(-c4cccc5c4oc4ccccc45)cc3)c3ccc(-n4c5ccccc5c5ccccc54)c(-c4ccc5c(c4)c4ccccc4n5-c4ccccc4)c3)cc2)cc1 ; c1ccc(-c2ccc(N(c3ccc(-c4cccc5c4oc4ccccc45)cc3)c3ccc(-n4c5ccccc5c5cccnc54)c(-c4ccccc4)c3)cc2)cc1 ; c1ccc(-c2ccc(N(c3ccc(-c4cccc5c4oc4ccccc45)cc3)c3ccc(-n4c5ccccc5c5cnccc54)c(-c4ccccc4)c3)cc2)cc1]

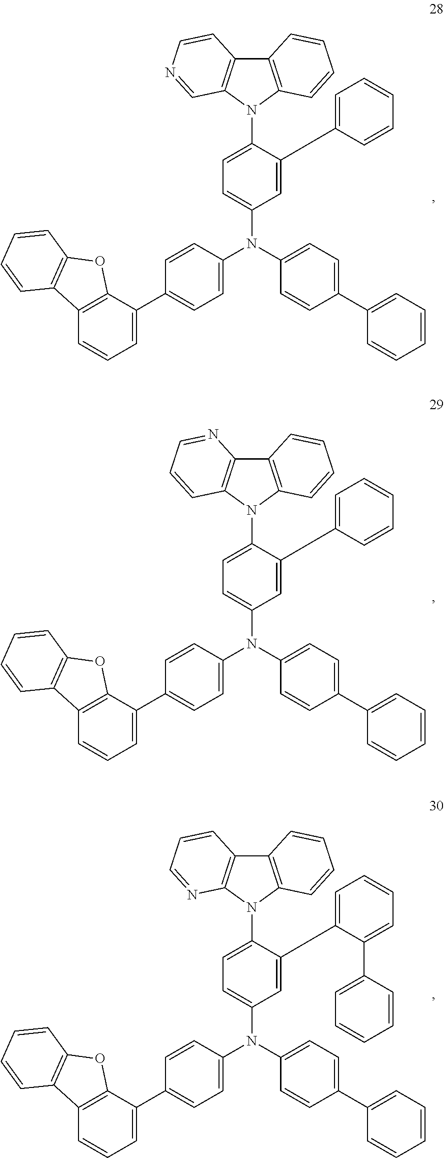 [SMILES: c1ccc(-c2ccc(N(c3ccc(-c4cccc5c4oc4ccccc45)cc3)c3ccc(-n4c5ccccc5c5ccncc54)c(-c4ccccc4)c3)cc2)cc1 ; c1ccc(-c2ccc(N(c3ccc(-c4cccc5c4oc4ccccc45)cc3)c3ccc(-n4c5ccccc5c5ncccc54)c(-c4ccccc4)c3)cc2)cc1 ; c1ccc(-c2ccc(N(c3ccc(-c4cccc5c4oc4ccccc45)cc3)c3ccc(-n4c5ccccc5c5cccnc54)c(-c4ccccc4-c4ccccc4)c3)cc2)cc1]

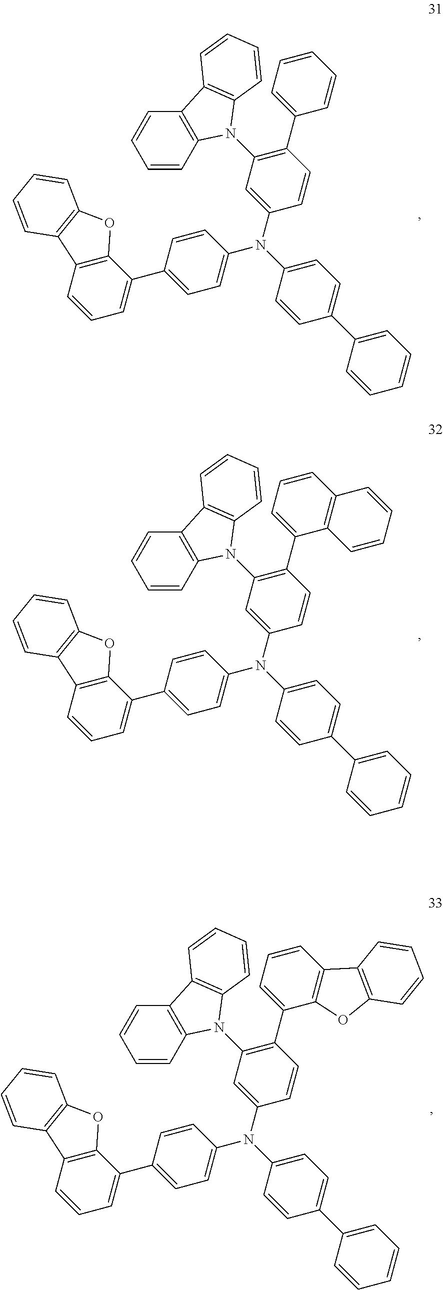 [SMILES: c1ccc(-c2ccc(N(c3ccc(-c4cccc5c4oc4ccccc45)cc3)c3ccc(-c4ccccc4)c(-n4c5ccccc5c5ccccc54)c3)cc2)cc1 ; c1ccc(-c2ccc(N(c3ccc(-c4cccc5c4oc4ccccc45)cc3)c3ccc(-c4cccc5ccccc45)c(-n4c5ccccc5c5ccccc54)c3)cc2)cc1 ; c1ccc(-c2ccc(N(c3ccc(-c4cccc5c4oc4ccccc45)cc3)c3ccc(-c4cccc5c4oc4ccccc45)c(-n4c5ccccc5c5ccccc54)c3)cc2)cc1]

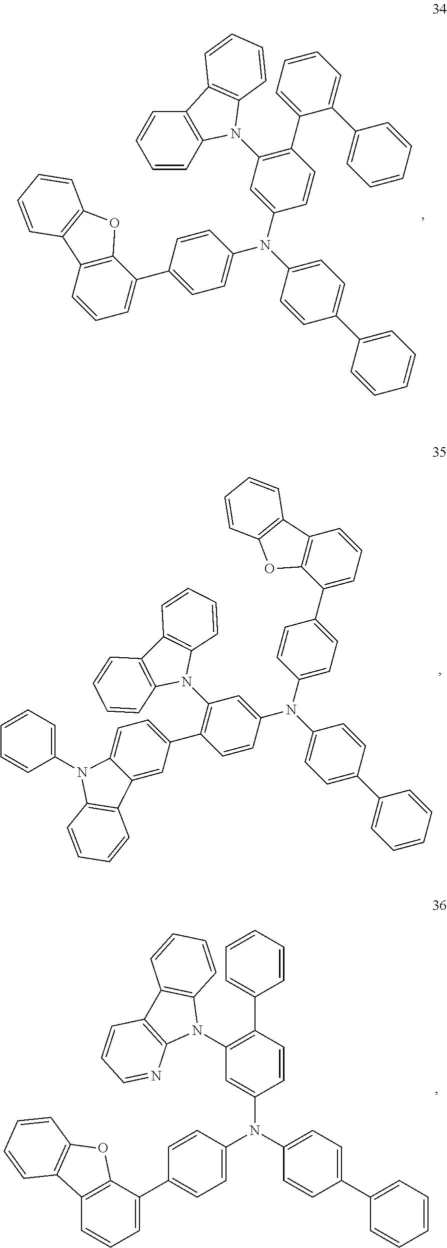 [SMILES: c1ccc(-c2ccc(N(c3ccc(-c4cccc5c4oc4ccccc45)cc3)c3ccc(-c4ccccc4-c4ccccc4)c(-n4c5ccccc5c5ccccc54)c3)cc2)cc1 ; c1ccc(-c2ccc(N(c3ccc(-c4cccc5c4oc4ccccc45)cc3)c3ccc(-c4ccc5c(c4)c4ccccc4n5-c4ccccc4)c(-n4c5ccccc5c5ccccc54)c3)cc2)cc1 ; c1ccc(-c2ccc(N(c3ccc(-c4cccc5c4oc4ccccc45)cc3)c3ccc(-c4ccccc4)c(-n4c5ccccc5c5cccnc54)c3)cc2)cc1]

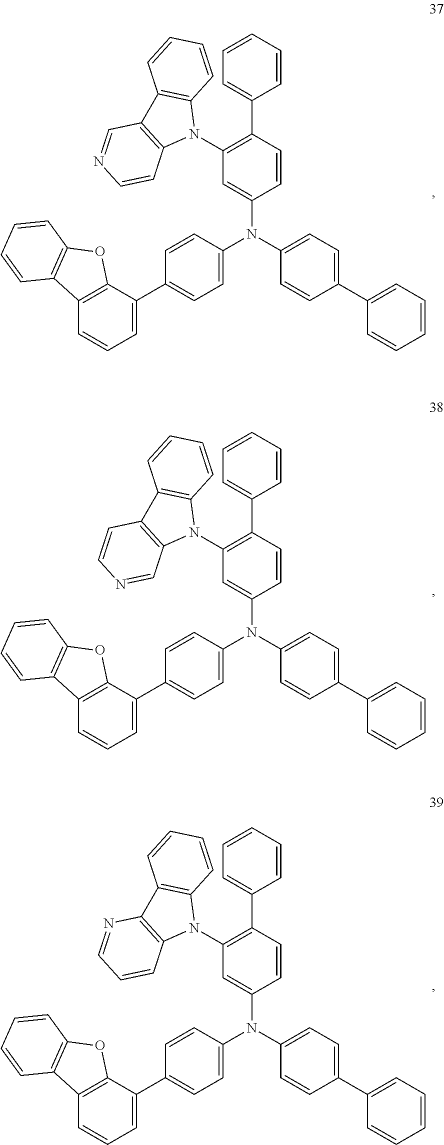 [SMILES: c1ccc(-c2ccc(N(c3ccc(-c4cccc5c4oc4ccccc45)cc3)c3ccc(-c4ccccc4)c(-n4c5ccccc5c5cnccc54)c3)cc2)cc1 ; c1ccc(-c2ccc(N(c3ccc(-c4cccc5c4oc4ccccc45)cc3)c3ccc(-c4ccccc4)c(-n4c5ccccc5c5ccncc54)c3)cc2)cc1 ; c1ccc(-c2ccc(N(c3ccc(-c4cccc5c4oc4ccccc45)cc3)c3ccc(-c4ccccc4)c(-n4c5ccccc5c5ncccc54)c3)cc2)cc1]

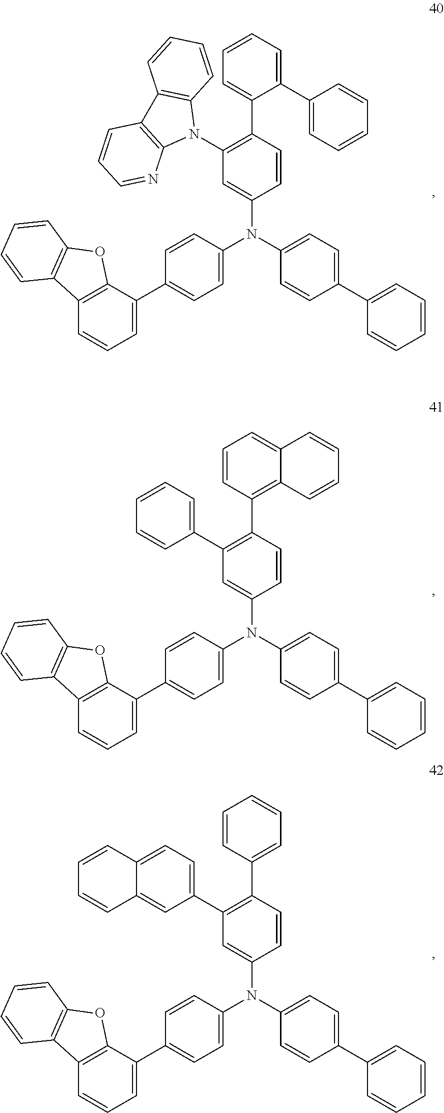 [SMILES: c1ccc(-c2ccc(N(c3ccc(-c4cccc5c4oc4ccccc45)cc3)c3ccc(-c4ccccc4-c4ccccc4)c(-n4c5ccccc5c5cccnc54)c3)cc2)cc1 ; c1ccc(-c2ccc(N(c3ccc(-c4cccc5c4oc4ccccc45)cc3)c3ccc(-c4cccc5ccccc45)c(-c4ccccc4)c3)cc2)cc1 ; c1ccc(-c2ccc(N(c3ccc(-c4cccc5c4oc4ccccc45)cc3)c3ccc(-c4ccccc4)c(-c4ccc5ccccc5c4)c3)cc2)cc1]

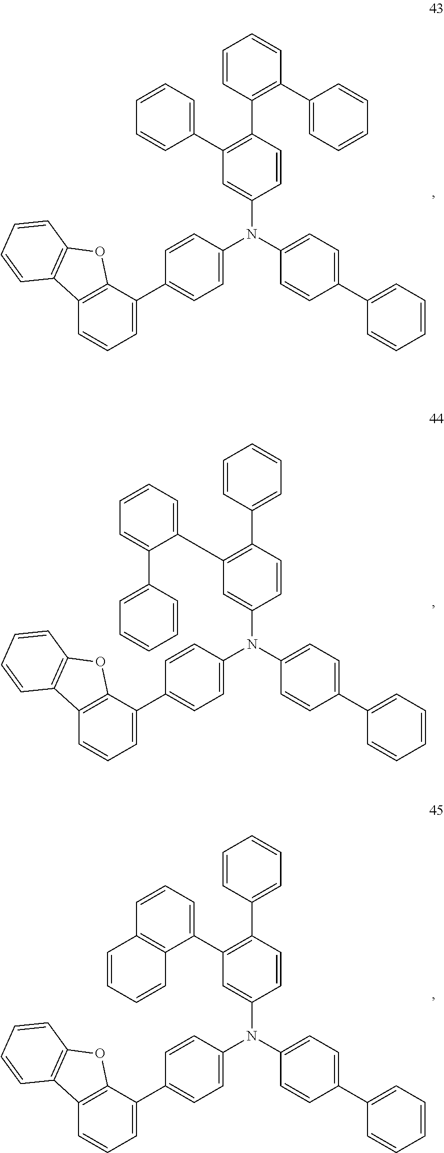 [SMILES: c1ccc(-c2ccc(N(c3ccc(-c4cccc5c4oc4ccccc45)cc3)c3ccc(-c4ccccc4-c4ccccc4)c(-c4ccccc4)c3)cc2)cc1 ; c1ccc(-c2ccc(N(c3ccc(-c4cccc5c4oc4ccccc45)cc3)c3ccc(-c4ccccc4)c(-c4ccccc4-c4ccccc4)c3)cc2)cc1 ; c1ccc(-c2ccc(N(c3ccc(-c4cccc5c4oc4ccccc45)cc3)c3ccc(-c4ccccc4)c(-c4cccc5ccccc45)c3)cc2)cc1]

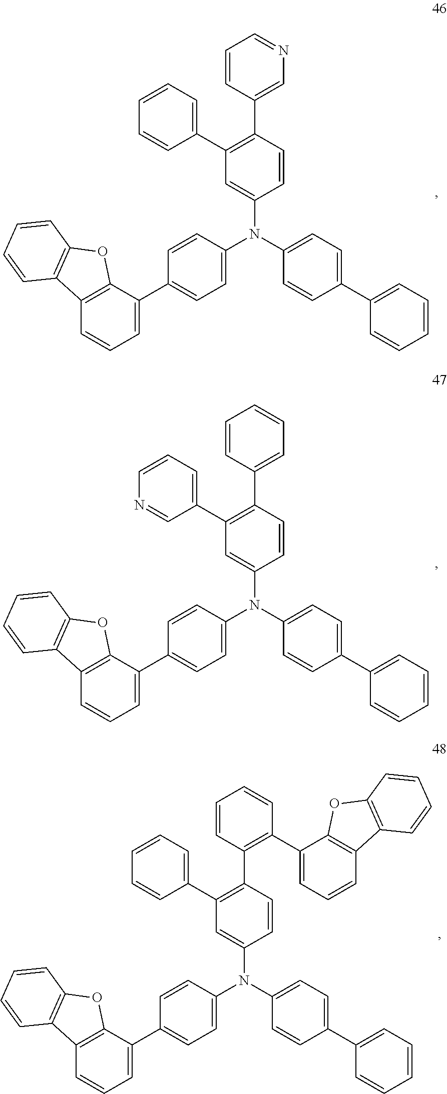 [SMILES: c1ccc(-c2ccc(N(c3ccc(-c4cccc5c4oc4ccccc45)cc3)c3ccc(-c4cccnc4)c(-c4ccccc4)c3)cc2)cc1 ; c1ccc(-c2ccc(N(c3ccc(-c4cccc5c4oc4ccccc45)cc3)c3ccc(-c4ccccc4)c(-c4cccnc4)c3)cc2)cc1 ; c1ccc(-c2ccc(N(c3ccc(-c4cccc5c4oc4ccccc45)cc3)c3ccc(-c4ccccc4-c4cccc5c4oc4ccccc45)c(-c4ccccc4)c3)cc2)cc1]

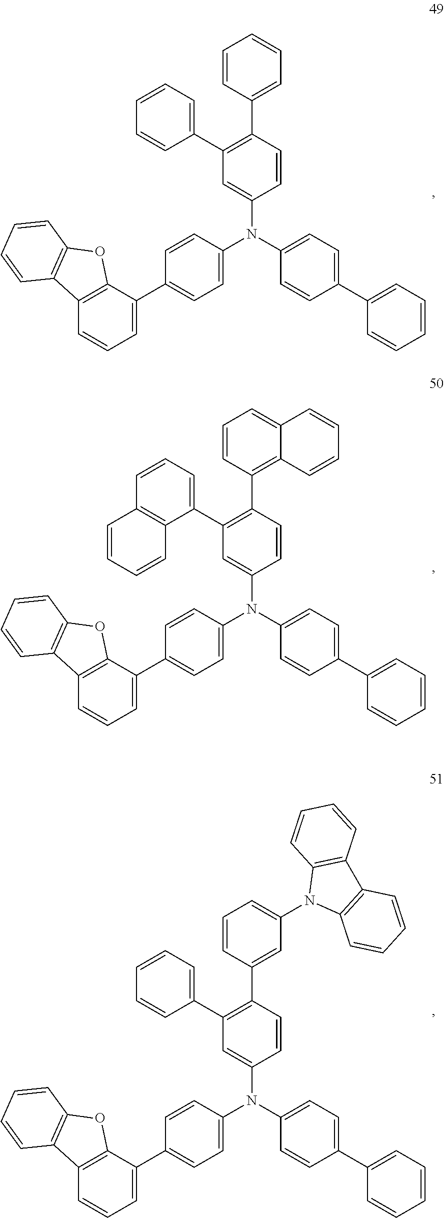 [SMILES: c1ccc(-c2ccc(N(c3ccc(-c4cccc5c4oc4ccccc45)cc3)c3ccc(-c4ccccc4)c(-c4ccccc4)c3)cc2)cc1 ; c1ccc(-c2ccc(N(c3ccc(-c4cccc5c4oc4ccccc45)cc3)c3ccc(-c4cccc5ccccc45)c(-c4cccc5ccccc45)c3)cc2)cc1 ; c1ccc(-c2ccc(N(c3ccc(-c4cccc5c4oc4ccccc45)cc3)c3ccc(-c4cccc(-n5c6ccccc6c6ccccc65)c4)c(-c4ccccc4)c3)cc2)cc1]

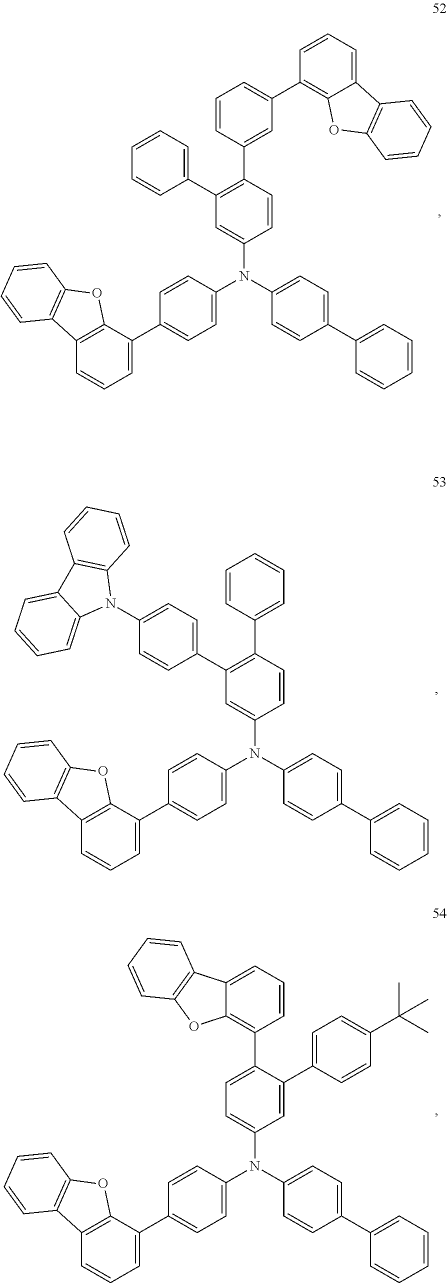 [SMILES: c1ccc(-c2ccc(N(c3ccc(-c4cccc5c4oc4ccccc45)cc3)c3ccc(-c4cccc(-c5cccc6c5oc5ccccc56)c4)c(-c4ccccc4)c3)cc2)cc1 ; c1ccc(-c2ccc(N(c3ccc(-c4cccc5c4oc4ccccc45)cc3)c3ccc(-c4ccccc4)c(-c4ccc(-n5c6ccccc6c6ccccc65)cc4)c3)cc2)cc1 ; CC(C)(C)c1ccc(-c2cc(N(c3ccc(-c4ccccc4)cc3)c3ccc(-c4cccc5c4oc4ccccc45)cc3)ccc2-c2cccc3c2oc2ccccc23)cc1]

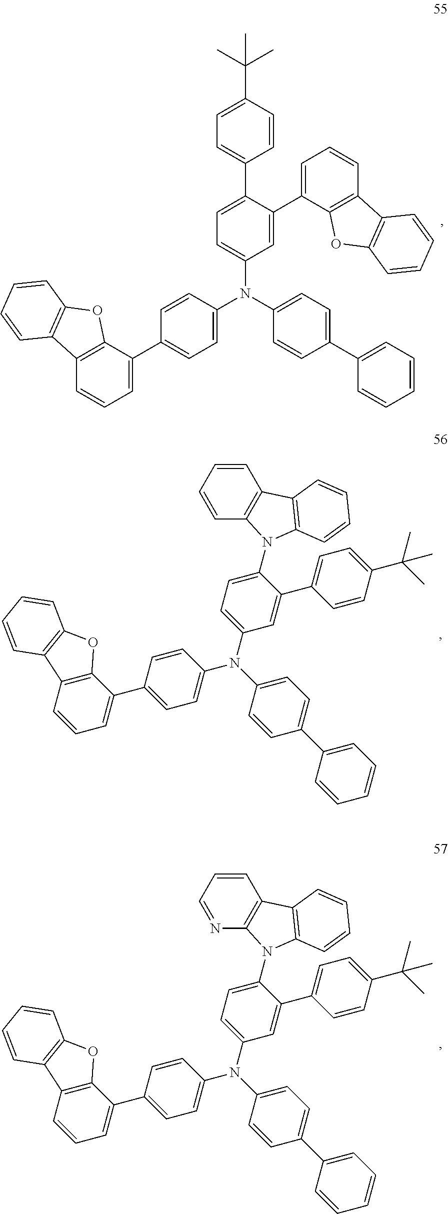 [SMILES: CC(C)(C)c1ccc(-c2ccc(N(c3ccc(-c4ccccc4)cc3)c3ccc(-c4cccc5c4oc4ccccc45)cc3)cc2-c2cccc3c2oc2ccccc23)cc1 ; CC(C)(C)c1ccc(-c2cc(N(c3ccc(-c4ccccc4)cc3)c3ccc(-c4cccc5c4oc4ccccc45)cc3)ccc2-n2c3ccccc3c3ccccc32)cc1 ; CC(C)(C)c1ccc(-c2cc(N(c3ccc(-c4ccccc4)cc3)c3ccc(-c4cccc5c4oc4ccccc45)cc3)ccc2-n2c3ccccc3c3cccnc32)cc1]

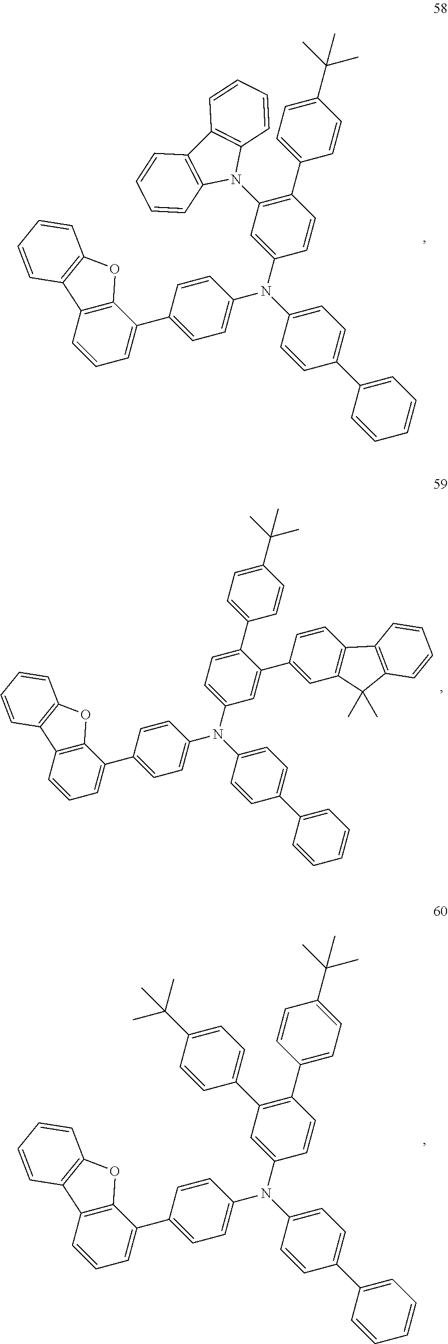 [SMILES: CC(C)(C)c1ccc(-c2ccc(N(c3ccc(-c4ccccc4)cc3)c3ccc(-c4cccc5c4oc4ccccc45)cc3)cc2-n2c3ccccc3c3ccccc32)cc1 ; CC(C)(C)c1ccc(-c2ccc(N(c3ccc(-c4ccccc4)cc3)c3ccc(-c4cccc5c4oc4ccccc45)cc3)cc2-c2ccc3c(c2)C(C)(C)c2ccccc2-3)cc1 ; CC(C)(C)c1ccc(-c2ccc(N(c3ccc(-c4ccccc4)cc3)c3ccc(-c4cccc5c4oc4ccccc45)cc3)cc2-c2ccc(C(C)(C)C)cc2)cc1]

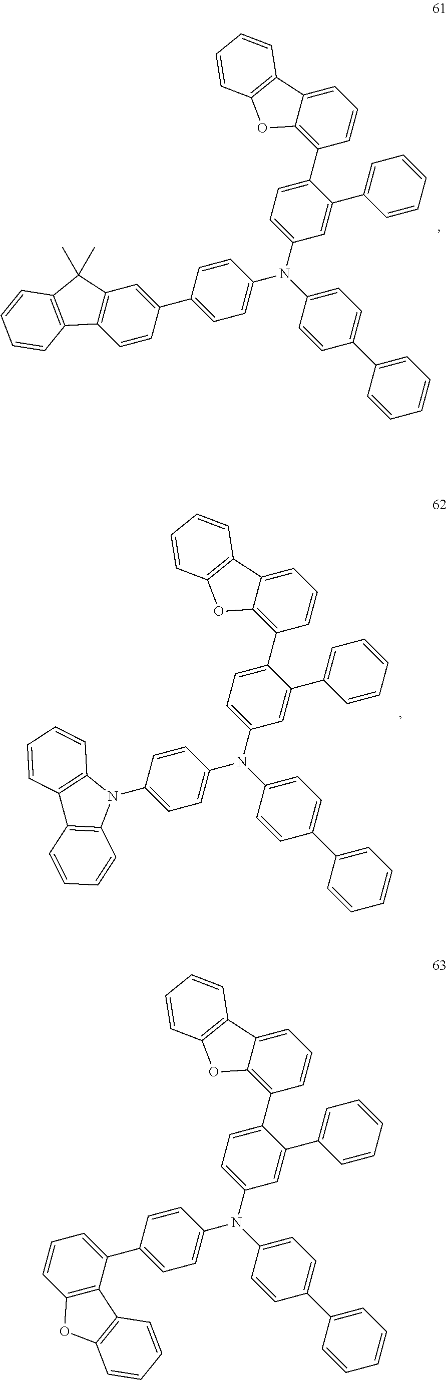 [SMILES: CC1(C)c2ccccc2-c2ccc(-c3ccc(N(c4ccc(-c5ccccc5)cc4)c4ccc(-c5cccc6c5oc5ccccc56)c(-c5ccccc5)c4)cc3)cc21 ; c1ccc(-c2ccc(N(c3ccc(-n4c5ccccc5c5ccccc54)cc3)c3ccc(-c4cccc5c4oc4ccccc45)c(-c4ccccc4)c3)cc2)cc1 ; c1ccc(-c2ccc(N(c3ccc(-c4cccc5oc6ccccc6c45)cc3)c3ccc(-c4cccc5c4oc4ccccc45)c(-c4ccccc4)c3)cc2)cc1]

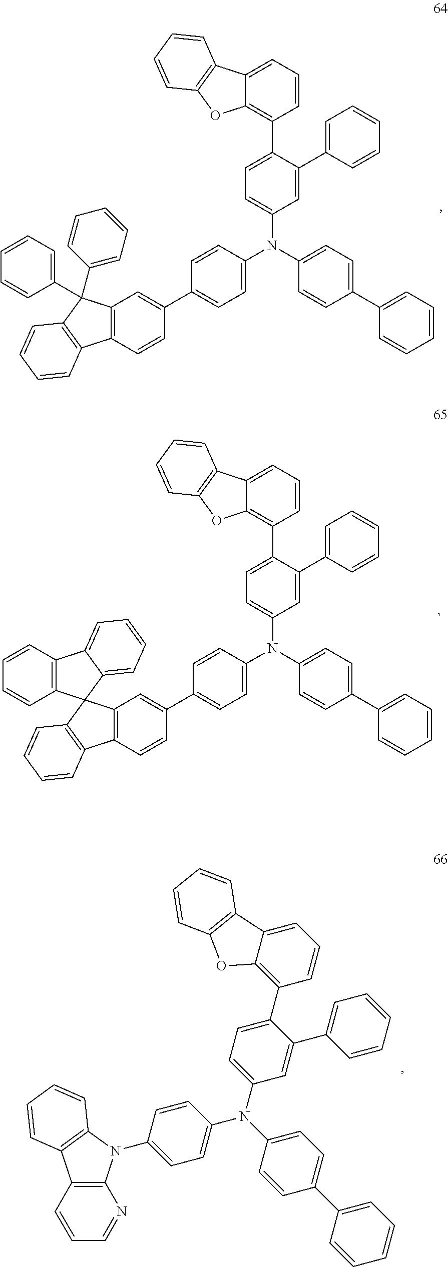 [SMILES: c1ccc(-c2ccc(N(c3ccc(-c4ccc5c(c4)C(c4ccccc4)(c4ccccc4)c4ccccc4-5)cc3)c3ccc(-c4cccc5c4oc4ccccc45)c(-c4ccccc4)c3)cc2)cc1 ; c1ccc(-c2ccc(N(c3ccc(-c4ccc5c(c4)C4(c6ccccc6-c6ccccc64)c4ccccc4-5)cc3)c3ccc(-c4cccc5c4oc4ccccc45)c(-c4ccccc4)c3)cc2)cc1 ; c1ccc(-c2ccc(N(c3ccc(-n4c5ccccc5c5cccnc54)cc3)c3ccc(-c4cccc5c4oc4ccccc45)c(-c4ccccc4)c3)cc2)cc1]

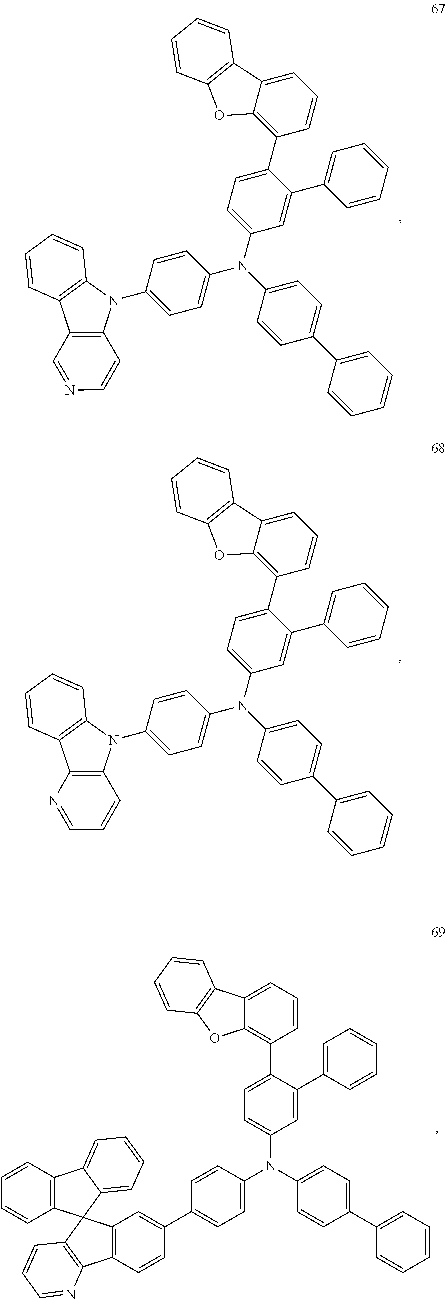 [SMILES: c1ccc(-c2ccc(N(c3ccc(-n4c5ccccc5c5cnccc54)cc3)c3ccc(-c4cccc5c4oc4ccccc45)c(-c4ccccc4)c3)cc2)cc1 ; c1ccc(-c2ccc(N(c3ccc(-n4c5ccccc5c5ncccc54)cc3)c3ccc(-c4cccc5c4oc4ccccc45)c(-c4ccccc4)c3)cc2)cc1 ; c1ccc(-c2ccc(N(c3ccc(-c4ccc5c(c4)C4(c6ccccc6-c6ccccc64)c4cccnc4-5)cc3)c3ccc(-c4cccc5c4oc4ccccc45)c(-c4ccccc4)c3)cc2)cc1]

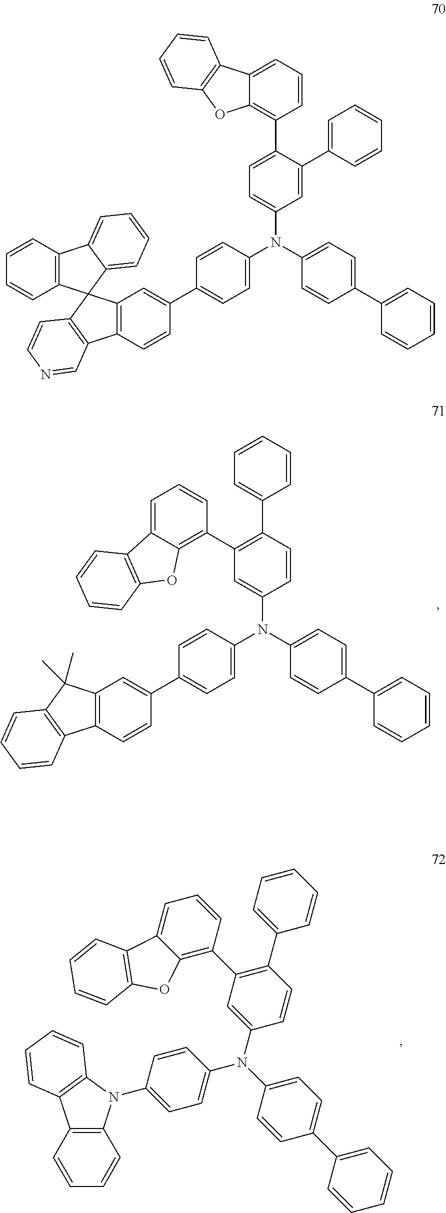 [SMILES: c1ccc(-c2ccc(N(c3ccc(-c4ccc5c(c4)C4(c6ccccc6-c6ccccc64)c4ccncc4-5)cc3)c3ccc(-c4cccc5c4oc4ccccc45)c(-c4ccccc4)c3)cc2)cc1 ; CC1(C)c2ccccc2-c2ccc(-c3ccc(N(c4ccc(-c5ccccc5)cc4)c4ccc(-c5ccccc5)c(-c5cccc6c5oc5ccccc56)c4)cc3)cc21 ; c1ccc(-c2ccc(N(c3ccc(-n4c5ccccc5c5ccccc54)cc3)c3ccc(-c4ccccc4)c(-c4cccc5c4oc4ccccc45)c3)cc2)cc1]

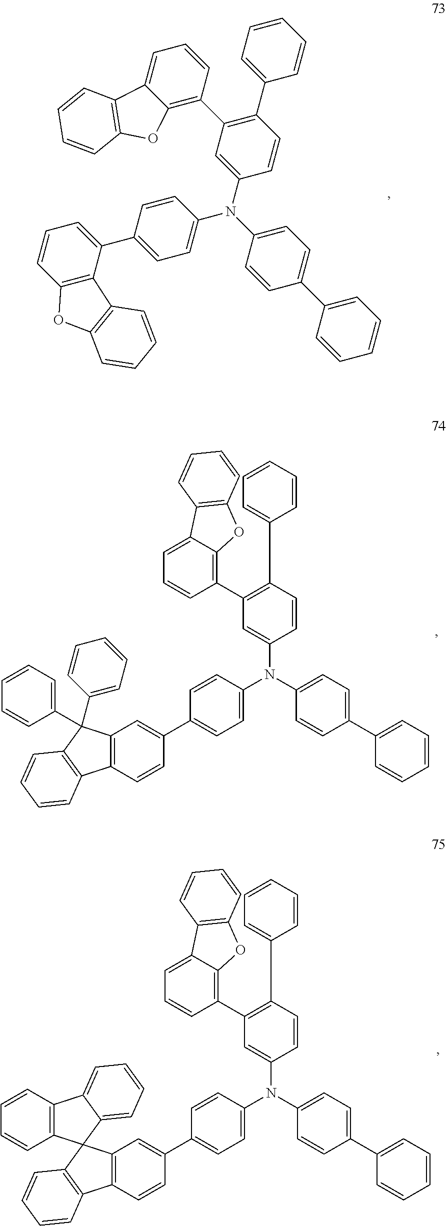 [SMILES: c1ccc(-c2ccc(N(c3ccc(-c4cccc5oc6ccccc6c45)cc3)c3ccc(-c4ccccc4)c(-c4cccc5c4oc4ccccc45)c3)cc2)cc1 ; c1ccc(-c2ccc(N(c3ccc(-c4ccc5c(c4)C(c4ccccc4)(c4ccccc4)c4ccccc4-5)cc3)c3ccc(-c4ccccc4)c(-c4cccc5c4oc4ccccc45)c3)cc2)cc1 ; c1ccc(-c2ccc(N(c3ccc(-c4ccc5c(c4)C4(c6ccccc6-c6ccccc64)c4ccccc4-5)cc3)c3ccc(-c4ccccc4)c(-c4cccc5c4oc4ccccc45)c3)cc2)cc1]

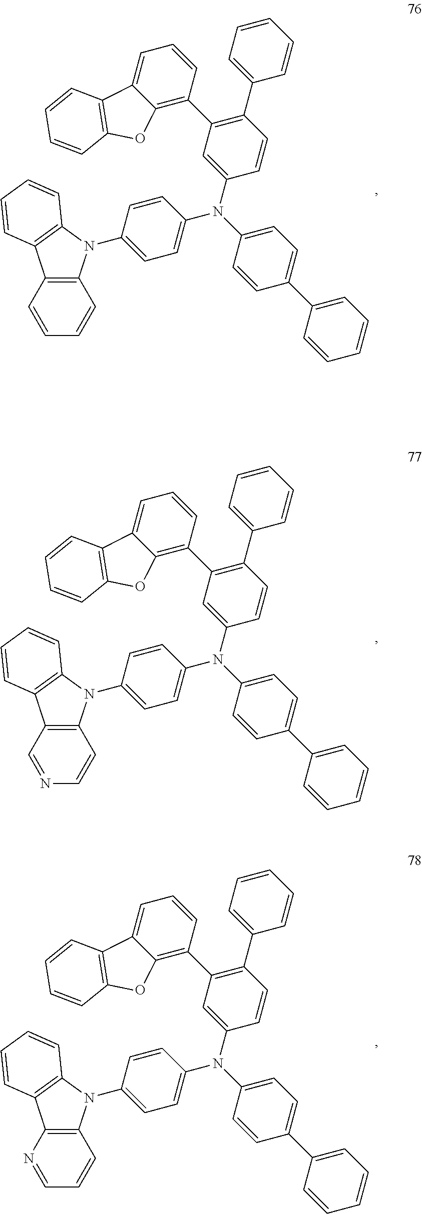 [SMILES: c1ccc(-c2ccc(N(c3ccc(-n4c5ccccc5c5ccccc54)cc3)c3ccc(-c4ccccc4)c(-c4cccc5c4oc4ccccc45)c3)cc2)cc1 ; c1ccc(-c2ccc(N(c3ccc(-n4c5ccccc5c5cnccc54)cc3)c3ccc(-c4ccccc4)c(-c4cccc5c4oc4ccccc45)c3)cc2)cc1 ; c1ccc(-c2ccc(N(c3ccc(-n4c5ccccc5c5ncccc54)cc3)c3ccc(-c4ccccc4)c(-c4cccc5c4oc4ccccc45)c3)cc2)cc1]

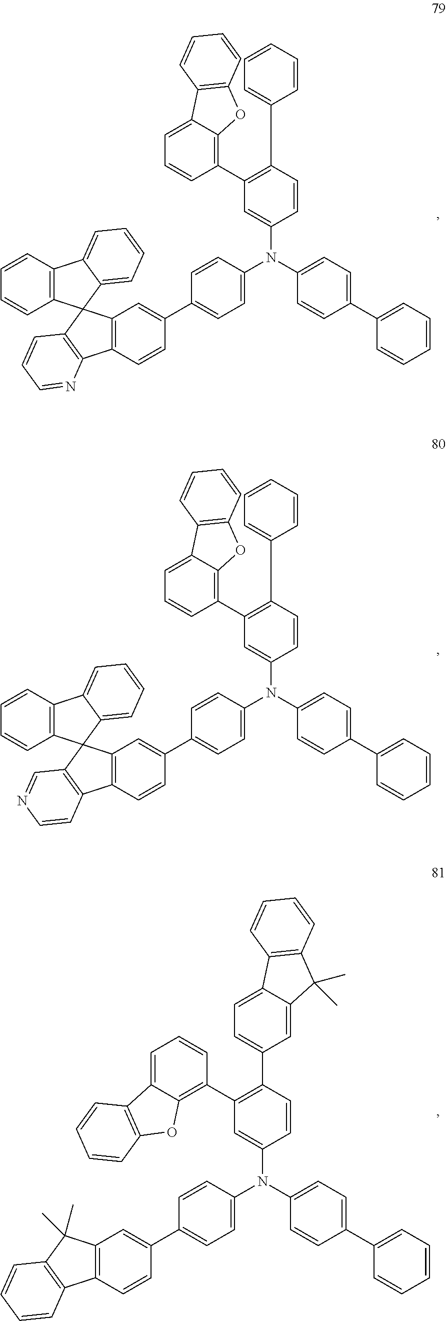 [SMILES: c1ccc(-c2ccc(N(c3ccc(-c4ccc5c(c4)C4(c6ccccc6-c6ccccc64)c4cccnc4-5)cc3)c3ccc(-c4ccccc4)c(-c4cccc5c4oc4ccccc45)c3)cc2)cc1 ; c1ccc(-c2ccc(N(c3ccc(-c4ccc5c(c4)C4(c6ccccc6-c6ccccc64)c4cnccc4-5)cc3)c3ccc(-c4ccccc4)c(-c4cccc5c4oc4ccccc45)c3)cc2)cc1 ; CC1(C)c2ccccc2-c2ccc(-c3ccc(N(c4ccc(-c5ccccc5)cc4)c4ccc(-c5ccc6c(c5)C(C)(C)c5ccccc5-6)c(-c5cccc6c5oc5ccccc56)c4)cc3)cc21]

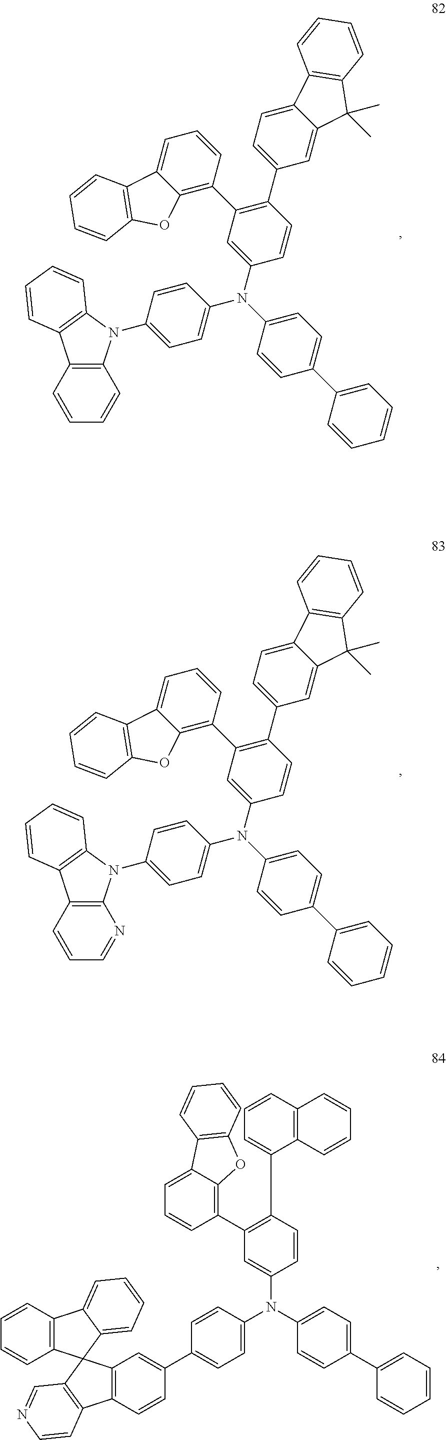 [SMILES: CC1(C)c2ccccc2-c2ccc(-c3ccc(N(c4ccc(-c5ccccc5)cc4)c4ccc(-n5c6ccccc6c6ccccc65)cc4)cc3-c3cccc4c3oc3ccccc34)cc21 ; CC1(C)c2ccccc2-c2ccc(-c3ccc(N(c4ccc(-c5ccccc5)cc4)c4ccc(-n5c6ccccc6c6cccnc65)cc4)cc3-c3cccc4c3oc3ccccc34)cc21 ; c1ccc(-c2ccc(N(c3ccc(-c4ccc5c(c4)C4(c6ccccc6-c6ccccc64)c4cnccc4-5)cc3)c3ccc(-c4cccc5ccccc45)c(-c4cccc5c4oc4ccccc45)c3)cc2)cc1]

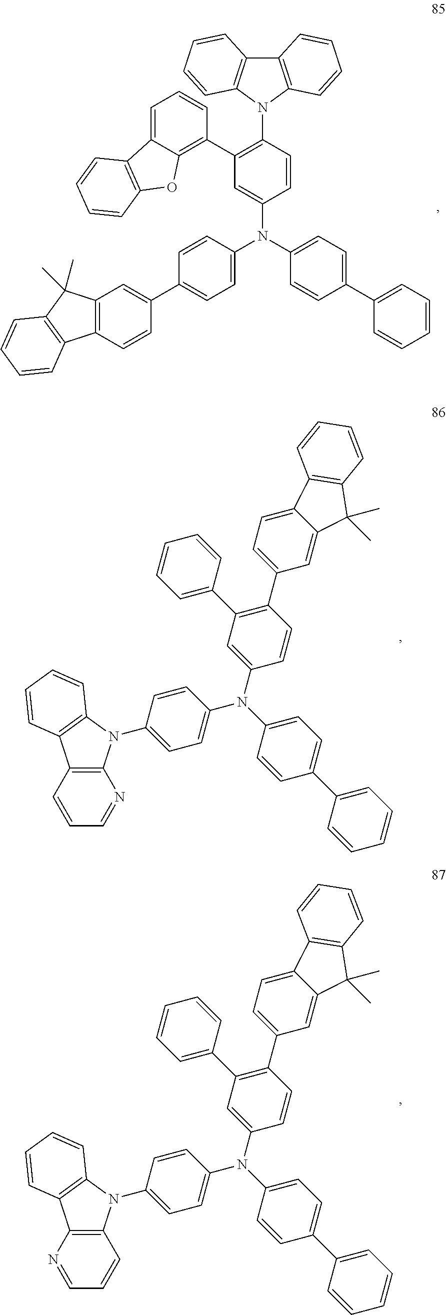

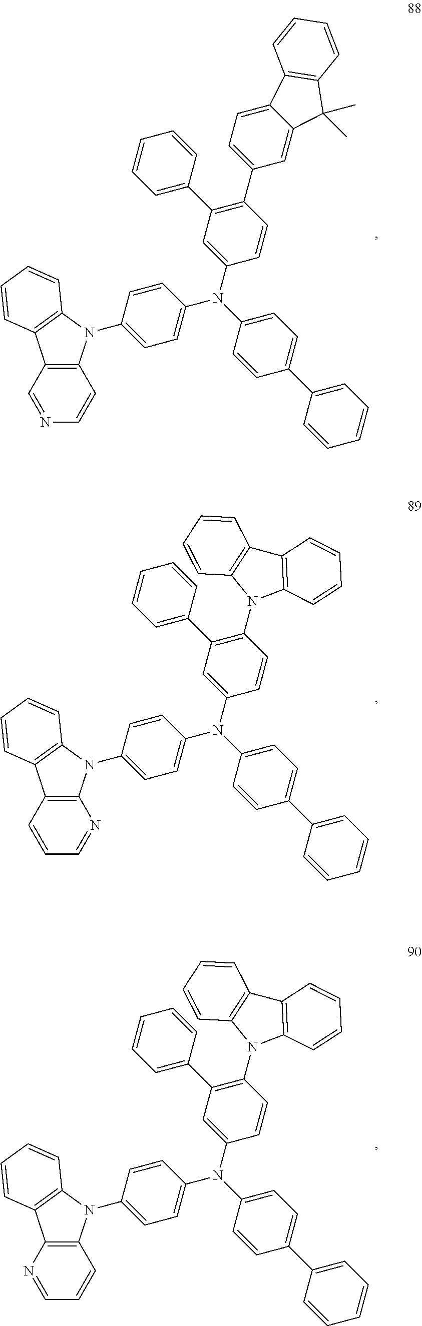 [SMILES: CC1(C)c2ccccc2-c2ccc(-c3ccc(N(c4ccc(-c5ccccc5)cc4)c4ccc(-n5c6ccccc6c6cnccc65)cc4)cc3-c3ccccc3)cc21 ; c1ccc(-c2ccc(N(c3ccc(-n4c5ccccc5c5cccnc54)cc3)c3ccc(-n4c5ccccc5c5ccccc54)c(-c4ccccc4)c3)cc2)cc1 ; c1ccc(-c2ccc(N(c3ccc(-n4c5ccccc5c5ncccc54)cc3)c3ccc(-n4c5ccccc5c5ccccc54)c(-c4ccccc4)c3)cc2)cc1]

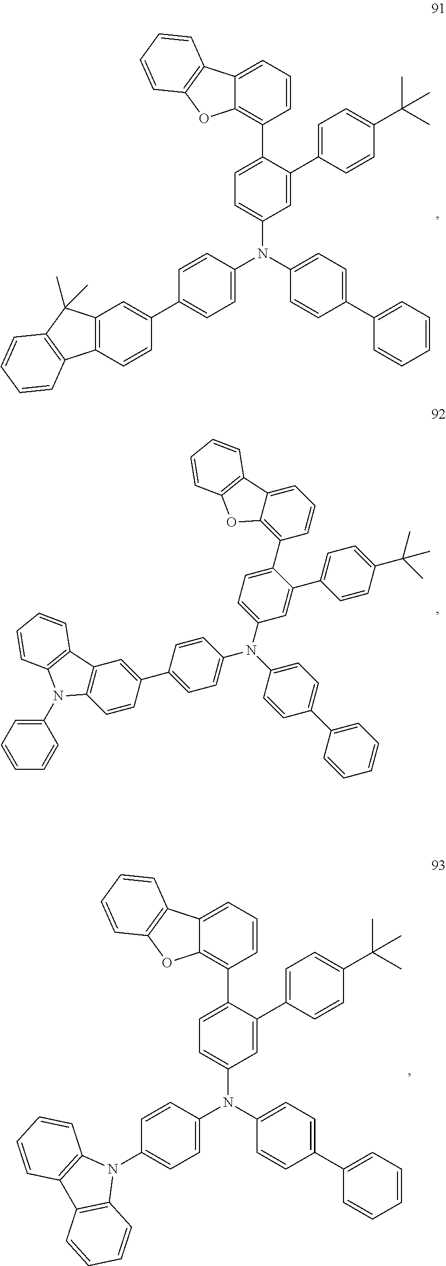 [SMILES: CC(C)(C)c1ccc(-c2cc(N(c3ccc(-c4ccccc4)cc3)c3ccc(-c4ccc5c(c4)C(C)(C)c4ccccc4-5)cc3)ccc2-c2cccc3c2oc2ccccc23)cc1 ; CC(C)(C)c1ccc(-c2cc(N(c3ccc(-c4ccccc4)cc3)c3ccc(-c4ccc5c(c4)c4ccccc4n5-c4ccccc4)cc3)ccc2-c2cccc3c2oc2ccccc23)cc1 ; CC(C)(C)c1ccc(-c2cc(N(c3ccc(-c4ccccc4)cc3)c3ccc(-n4c5ccccc5c5ccccc54)cc3)ccc2-c2cccc3c2oc2ccccc23)cc1]

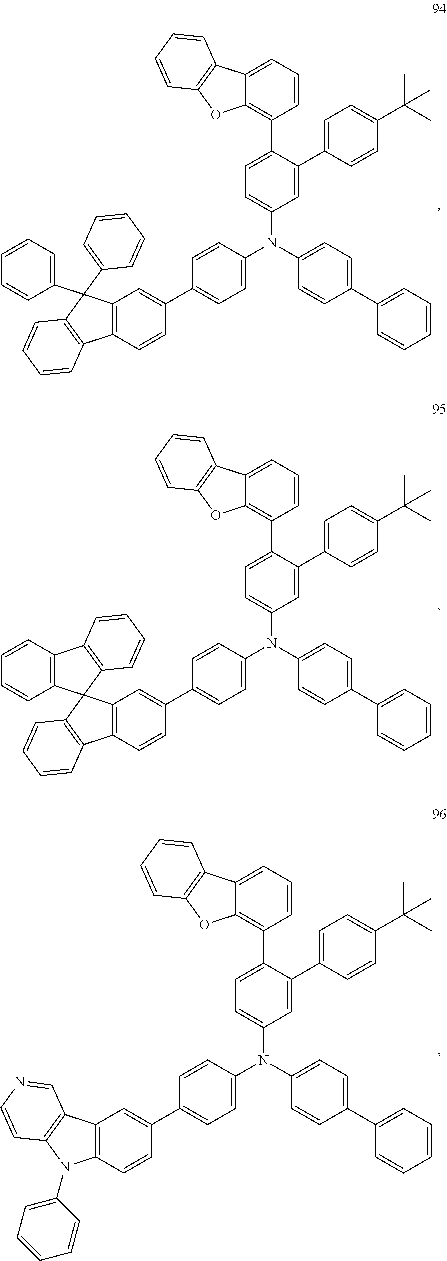 [SMILES: CC(C)(C)c1ccc(-c2cc(N(c3ccc(-c4ccccc4)cc3)c3ccc(-c4ccc5c(c4)C(c4ccccc4)(c4ccccc4)c4ccccc4-5)cc3)ccc2-c2cccc3c2oc2ccccc23)cc1 ; CC(C)(C)c1ccc(-c2cc(N(c3ccc(-c4ccccc4)cc3)c3ccc(-c4ccc5c(c4)C4(c6ccccc6-c6ccccc64)c4ccccc4-5)cc3)ccc2-c2cccc3c2oc2ccccc23)cc1 ; CC(C)(C)c1ccc(-c2cc(N(c3ccc(-c4ccccc4)cc3)c3ccc(-c4ccc5c(c4)c4cnccc4n5-c4ccccc4)cc3)ccc2-c2cccc3c2oc2ccccc23)cc1]

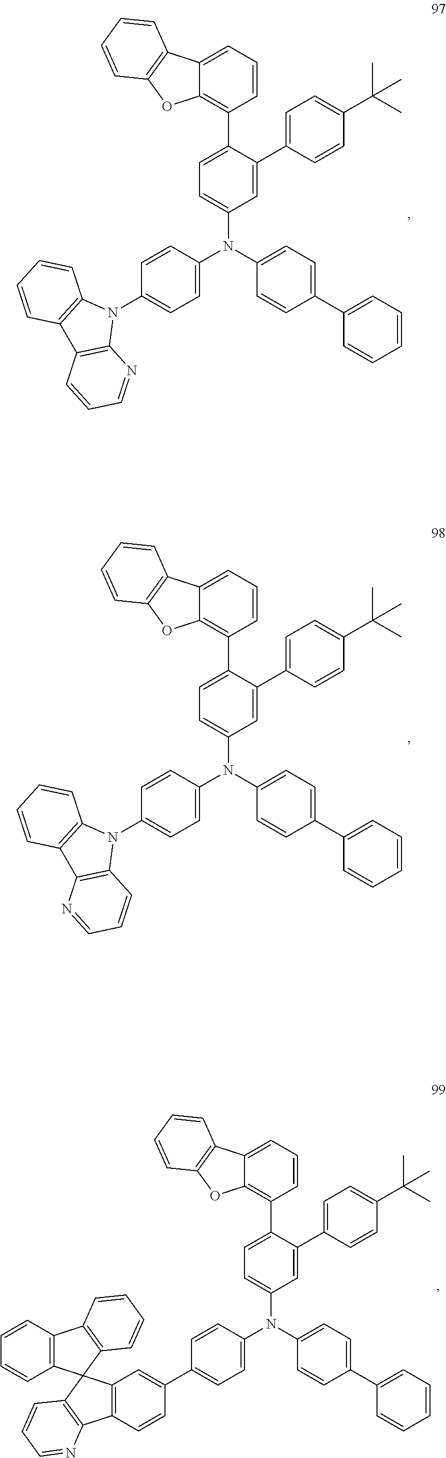 [SMILES: CC(C)(C)c1ccc(-c2cc(N(c3ccc(-c4ccccc4)cc3)c3ccc(-n4c5ccccc5c5cccnc54)cc3)ccc2-c2cccc3c2oc2ccccc23)cc1 ; CC(C)(C)c1ccc(-c2cc(N(c3ccc(-c4ccccc4)cc3)c3ccc(-n4c5ccccc5c5ncccc54)cc3)ccc2-c2cccc3c2oc2ccccc23)cc1 ; CC(C)(C)c1ccc(-c2cc(N(c3ccc(-c4ccccc4)cc3)c3ccc(-c4ccc5c(c4)C4(c6ccccc6-c6ccccc64)c4cccnc4-5)cc3)ccc2-c2cccc3c2oc2ccccc23)cc1]

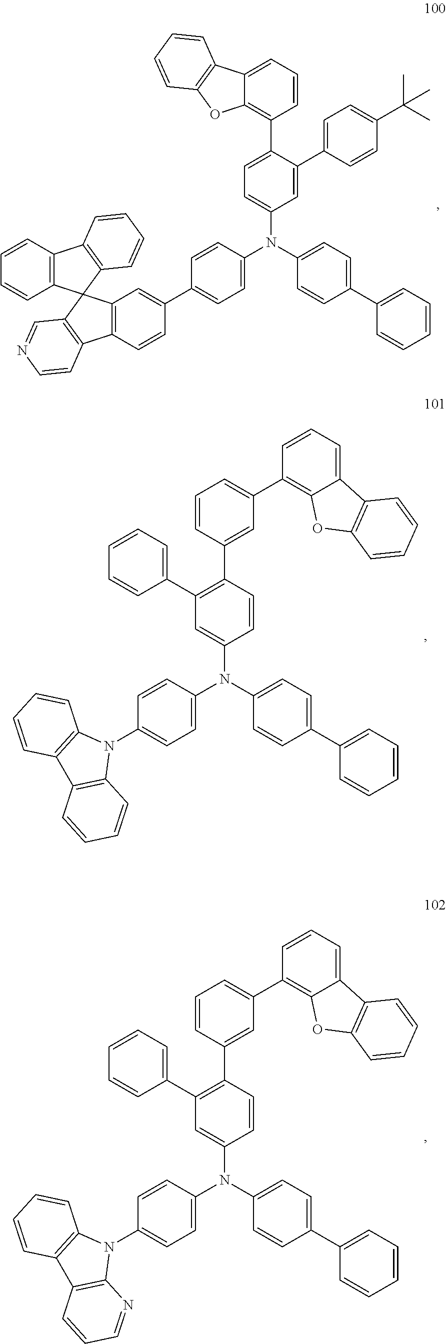 [SMILES: CC(C)(C)c1ccc(-c2cc(N(c3ccc(-c4ccccc4)cc3)c3ccc(-c4ccc5c(c4)C4(c6ccccc6-c6ccccc64)c4cnccc4-5)cc3)ccc2-c2cccc3c2oc2ccccc23)cc1 ; c1ccc(-c2ccc(N(c3ccc(-n4c5ccccc5c5ccccc54)cc3)c3ccc(-c4cccc(-c5cccc6c5oc5ccccc56)c4)c(-c4ccccc4)c3)cc2)cc1 ; c1ccc(-c2ccc(N(c3ccc(-n4c5ccccc5c5cccnc54)cc3)c3ccc(-c4cccc(-c5cccc6c5oc5ccccc56)c4)c(-c4ccccc4)c3)cc2)cc1]

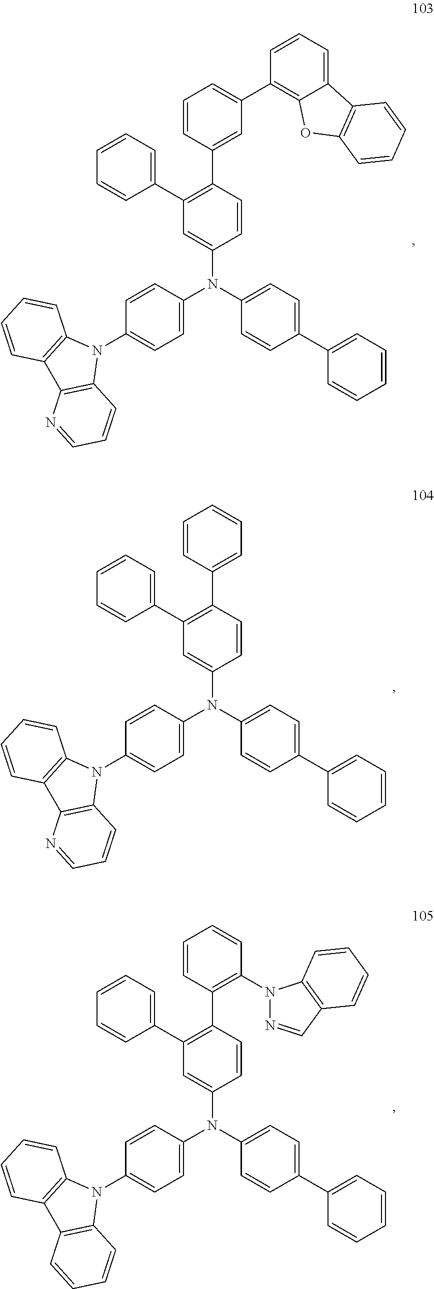 [SMILES: c1ccc(-c2ccc(N(c3ccc(-n4c5ccccc5c5ncccc54)cc3)c3ccc(-c4cccc(-c5cccc6c5oc5ccccc56)c4)c(-c4ccccc4)c3)cc2)cc1 ; c1ccc(-c2ccc(N(c3ccc(-n4c5ccccc5c5ncccc54)cc3)c3ccc(-c4ccccc4)c(-c4ccccc4)c3)cc2)cc1 ; c1ccc(-c2ccc(N(c3ccc(-n4c5ccccc5c5ccccc54)cc3)c3ccc(-c4ccccc4-n4ncc5ccccc54)c(-c4ccccc4)c3)cc2)cc1]

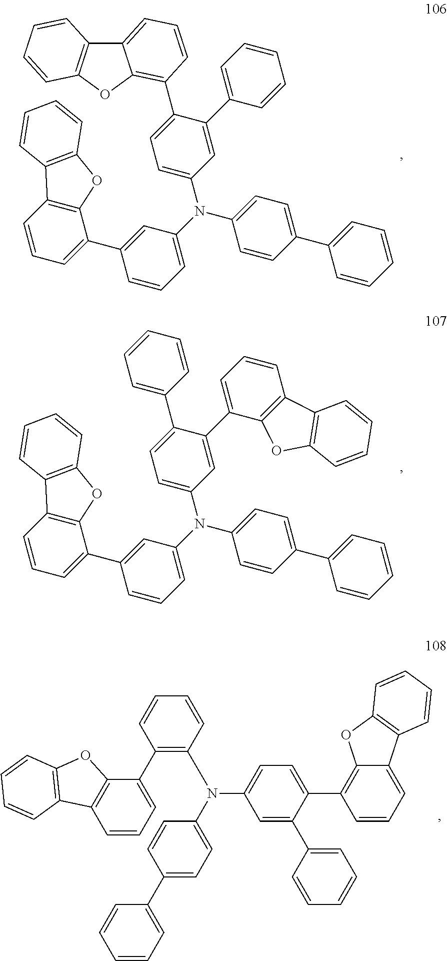

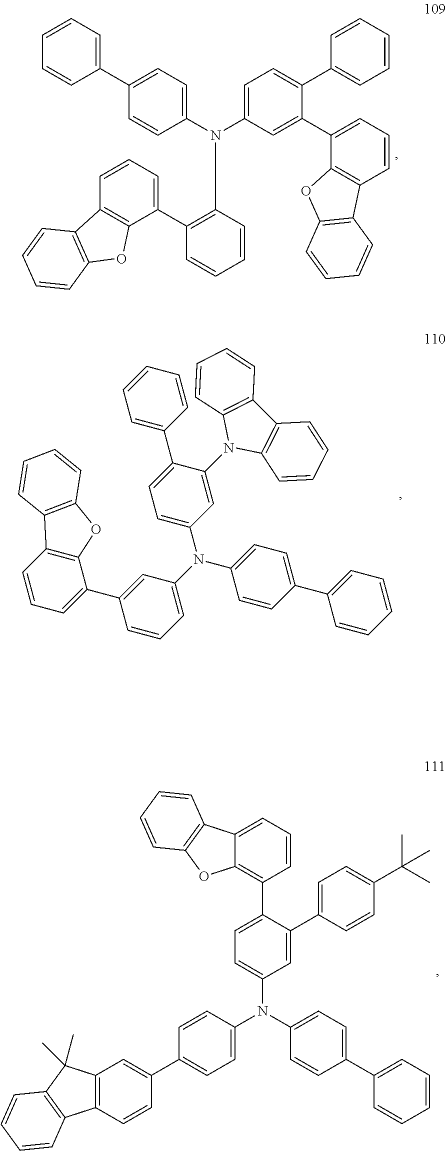 [SMILES: c1ccc(-c2ccc(N(c3ccc(-c4ccccc4)c(-c4cccc5c4oc4ccccc45)c3)c3ccccc3-c3cccc4c3oc3ccccc34)cc2)cc1 ; c1ccc(-c2ccc(N(c3cccc(-c4cccc5c4oc4ccccc45)c3)c3ccc(-c4ccccc4)c(-n4c5ccccc5c5ccccc54)c3)cc2)cc1 ; CC(C)(C)c1ccc(-c2cc(N(c3ccc(-c4ccccc4)cc3)c3ccc(-c4ccc5c(c4)C(C)(C)c4ccccc4-5)cc3)ccc2-c2cccc3c2oc2ccccc23)cc1]

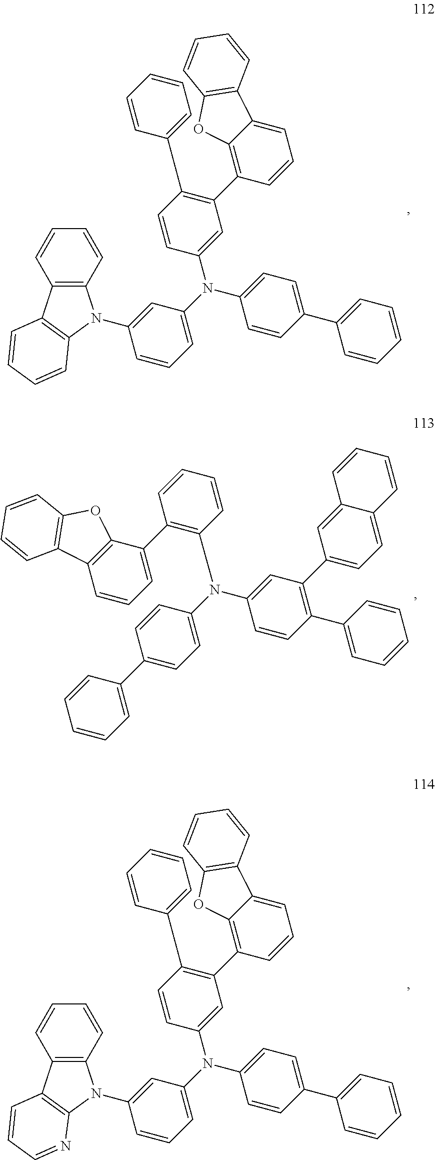 [SMILES: c1ccc(-c2ccc(N(c3cccc(-n4c5ccccc5c5ccccc54)c3)c3ccc(-c4ccccc4)c(-c4cccc5c4oc4ccccc45)c3)cc2)cc1 ; c1ccc(-c2ccc(N(c3ccc(-c4ccccc4)c(-c4ccc5ccccc5c4)c3)c3ccccc3-c3cccc4c3oc3ccccc34)cc2)cc1 ; c1ccc(-c2ccc(N(c3cccc(-n4c5ccccc5c5cccnc54)c3)c3ccc(-c4ccccc4)c(-c4cccc5c4oc4ccccc45)c3)cc2)cc1]

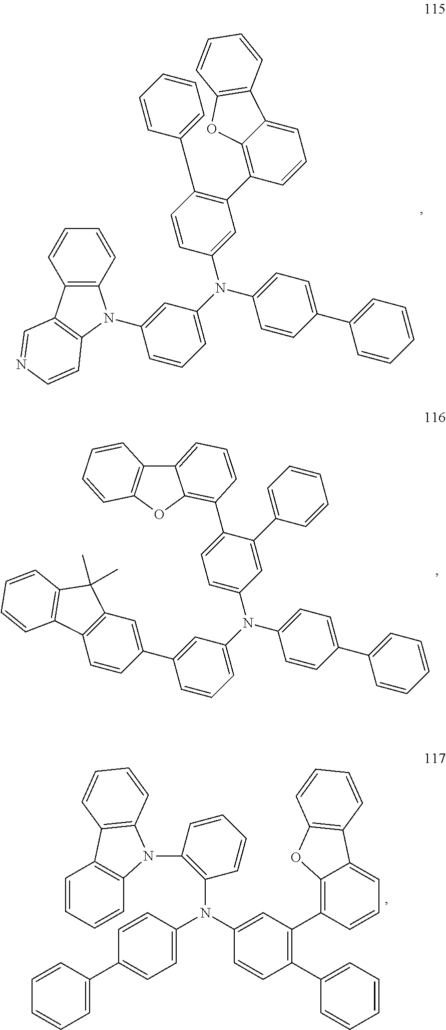 [SMILES: c1ccc(-c2ccc(N(c3cccc(-n4c5ccccc5c5cnccc54)c3)c3ccc(-c4ccccc4)c(-c4cccc5c4oc4ccccc45)c3)cc2)cc1 ; CC1(C)c2ccccc2-c2ccc(-c3cccc(N(c4ccc(-c5ccccc5)cc4)c4ccc(-c5cccc6c5oc5ccccc56)c(-c5ccccc5)c4)c3)cc21 ; c1ccc(-c2ccc(N(c3ccc(-c4ccccc4)c(-c4cccc5c4oc4ccccc45)c3)c3ccccc3-n3c4ccccc4c4ccccc43)cc2)cc1]

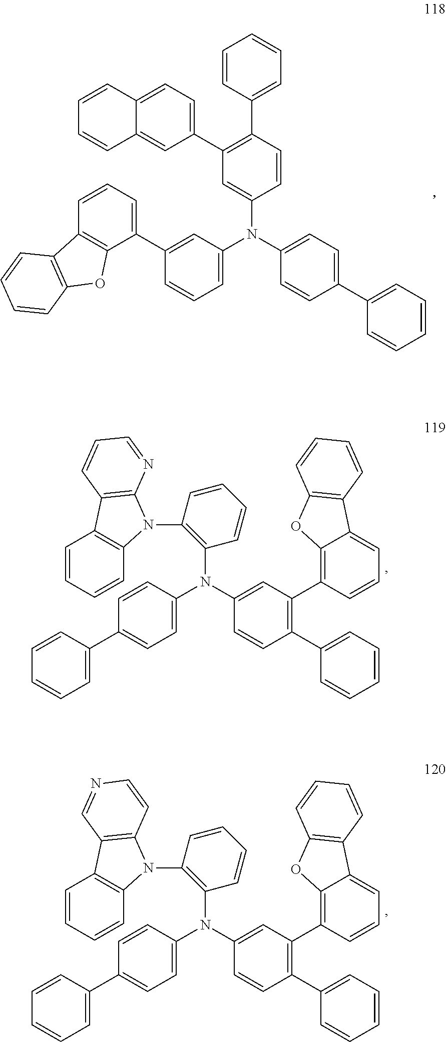 [SMILES: c1ccc(-c2ccc(N(c3cccc(-c4cccc5c4oc4ccccc45)c3)c3ccc(-c4ccccc4)c(-c4ccc5ccccc5c4)c3)cc2)cc1 ; c1ccc(-c2ccc(N(c3ccc(-c4ccccc4)c(-c4cccc5c4oc4ccccc45)c3)c3ccccc3-n3c4ccccc4c4cccnc43)cc2)cc1 ; c1ccc(-c2ccc(N(c3ccc(-c4ccccc4)c(-c4cccc5c4oc4ccccc45)c3)c3ccccc3-n3c4ccccc4c4cnccc43)cc2)cc1]

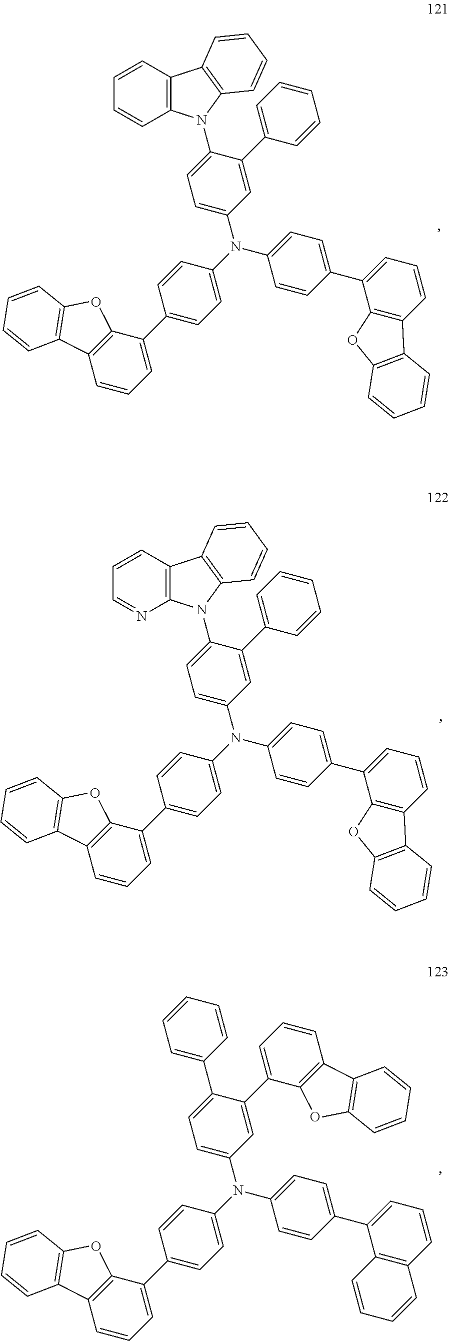 [SMILES: c1ccc(-c2cc(N(c3ccc(-c4cccc5c4oc4ccccc45)cc3)c3ccc(-c4cccc5c4oc4ccccc45)cc3)ccc2-n2c3ccccc3c3ccccc32)cc1 ; c1ccc(-c2cc(N(c3ccc(-c4cccc5c4oc4ccccc45)cc3)c3ccc(-c4cccc5c4oc4ccccc45)cc3)ccc2-n2c3ccccc3c3cccnc32)cc1 ; c1ccc(-c2ccc(N(c3ccc(-c4cccc5ccccc45)cc3)c3ccc(-c4cccc5c4oc4ccccc45)cc3)cc2-c2cccc3c2oc2ccccc23)cc1]

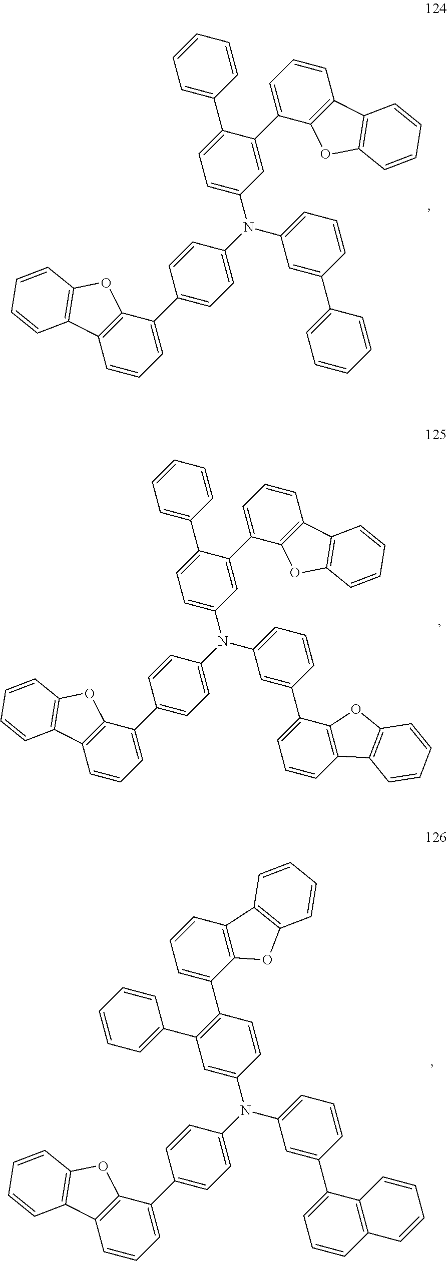 [SMILES: c1ccc(-c2cccc(N(c3ccc(-c4cccc5c4oc4ccccc45)cc3)c3ccc(-c4ccccc4)c(-c4cccc5c4oc4ccccc45)c3)c2)cc1 ; c1ccc(-c2ccc(N(c3ccc(-c4cccc5c4oc4ccccc45)cc3)c3cccc(-c4cccc5c4oc4ccccc45)c3)cc2-c2cccc3c2oc2ccccc23)cc1 ; c1ccc(-c2cc(N(c3ccc(-c4cccc5c4oc4ccccc45)cc3)c3cccc(-c4cccc5ccccc45)c3)ccc2-c2cccc3c2oc2ccccc23)cc1]

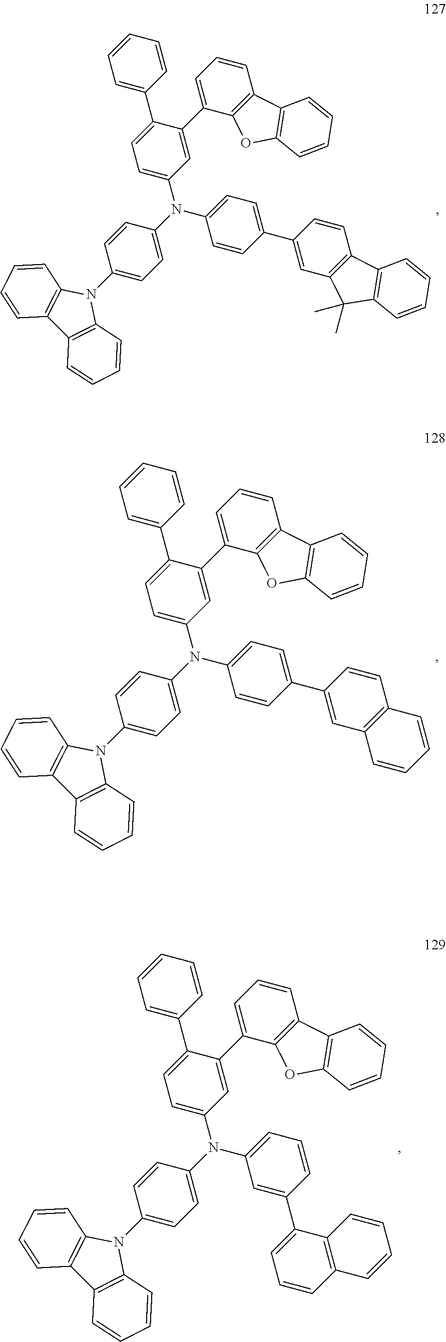 [SMILES: CC1(C)c2ccccc2-c2ccc(-c3ccc(N(c4ccc(-n5c6ccccc6c6ccccc65)cc4)c4ccc(-c5ccccc5)c(-c5cccc6c5oc5ccccc56)c4)cc3)cc21 ; c1ccc(-c2ccc(N(c3ccc(-c4ccc5ccccc5c4)cc3)c3ccc(-n4c5ccccc5c5ccccc54)cc3)cc2-c2cccc3c2oc2ccccc23)cc1 ; c1ccc(-c2ccc(N(c3ccc(-n4c5ccccc5c5ccccc54)cc3)c3cccc(-c4cccc5ccccc45)c3)cc2-c2cccc3c2oc2ccccc23)cc1]

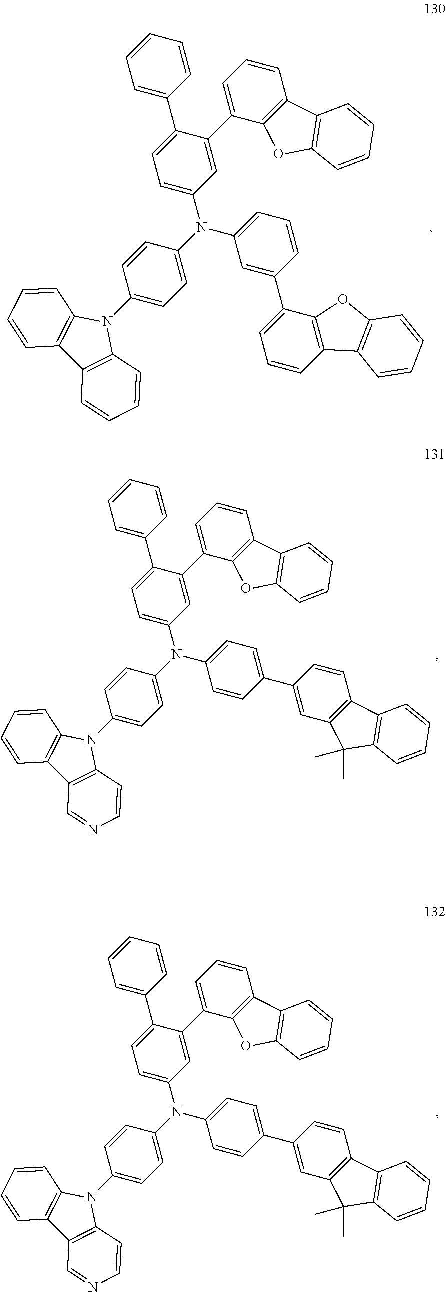 [SMILES: c1ccc(-c2ccc(N(c3ccc(-n4c5ccccc5c5ccccc54)cc3)c3cccc(-c4cccc5c4oc4ccccc45)c3)cc2-c2cccc3c2oc2ccccc23)cc1 ; CC1(C)c2ccccc2-c2ccc(-c3ccc(N(c4ccc(-n5c6ccccc6c6cnccc65)cc4)c4ccc(-c5ccccc5)c(-c5cccc6c5oc5ccccc56)c4)cc3)cc21 ; CC1(C)c2ccccc2-c2ccc(-c3ccc(N(c4ccc(-n5c6ccccc6c6cnccc65)cc4)c4ccc(-c5ccccc5)c(-c5cccc6c5oc5ccccc56)c4)cc3)cc21]

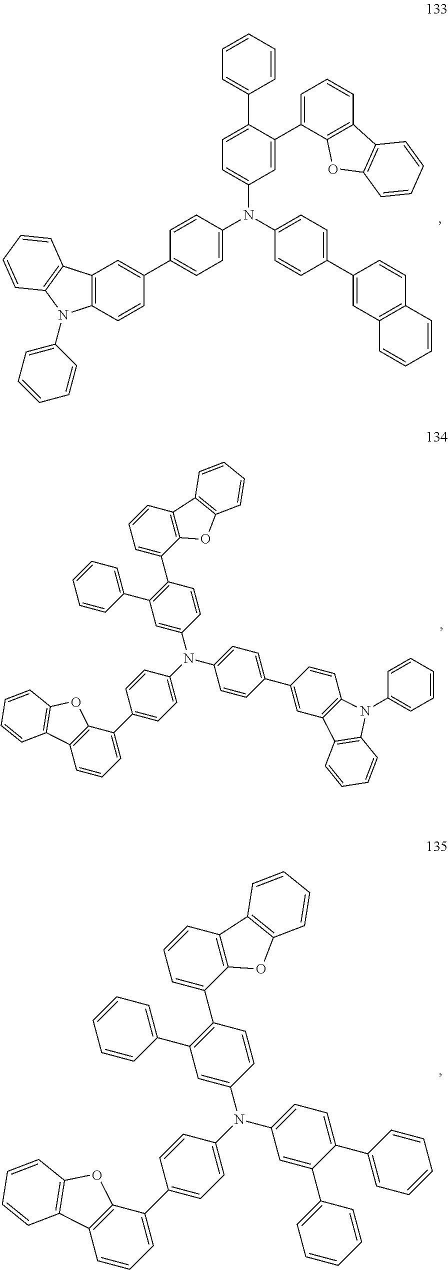 [SMILES: c1ccc(-c2ccc(N(c3ccc(-c4ccc5ccccc5c4)cc3)c3ccc(-c4ccc5c(c4)c4ccccc4n5-c4ccccc4)cc3)cc2-c2cccc3c2oc2ccccc23)cc1 ; c1ccc(-c2cc(N(c3ccc(-c4ccc5c(c4)c4ccccc4n5-c4ccccc4)cc3)c3ccc(-c4cccc5c4oc4ccccc45)cc3)ccc2-c2cccc3c2oc2ccccc23)cc1 ; c1ccc(-c2ccc(N(c3ccc(-c4cccc5c4oc4ccccc45)cc3)c3ccc(-c4cccc5c4oc4ccccc45)c(-c4ccccc4)c3)cc2-c2ccccc2)cc1]

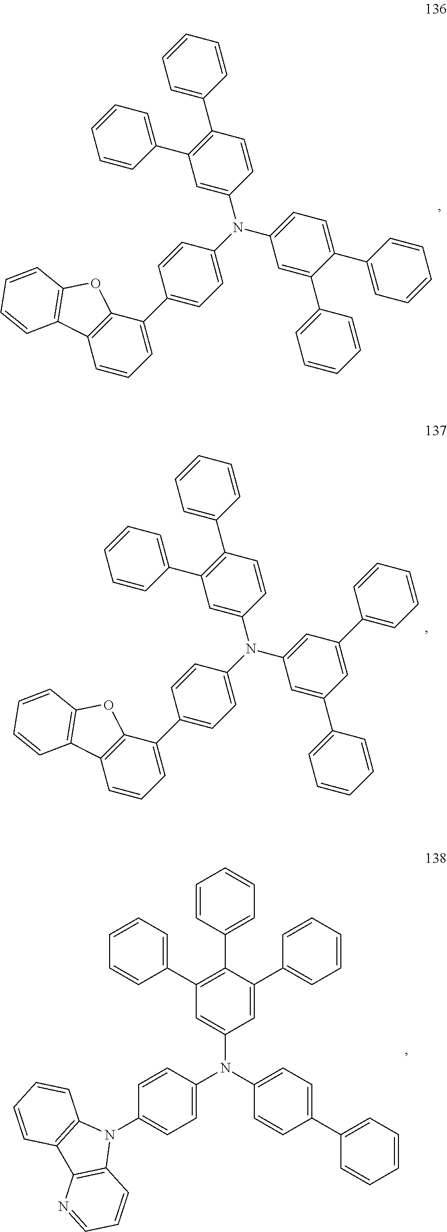 [SMILES: c1ccc(-c2ccc(N(c3ccc(-c4cccc5c4oc4ccccc45)cc3)c3ccc(-c4ccccc4)c(-c4ccccc4)c3)cc2-c2ccccc2)cc1 ; c1ccc(-c2cc(-c3ccccc3)cc(N(c3ccc(-c4cccc5c4oc4ccccc45)cc3)c3ccc(-c4ccccc4)c(-c4ccccc4)c3)c2)cc1 ; c1ccc(-c2ccc(N(c3ccc(-n4c5ccccc5c5ncccc54)cc3)c3cc(-c4ccccc4)c(-c4ccccc4)c(-c4ccccc4)c3)cc2)cc1]

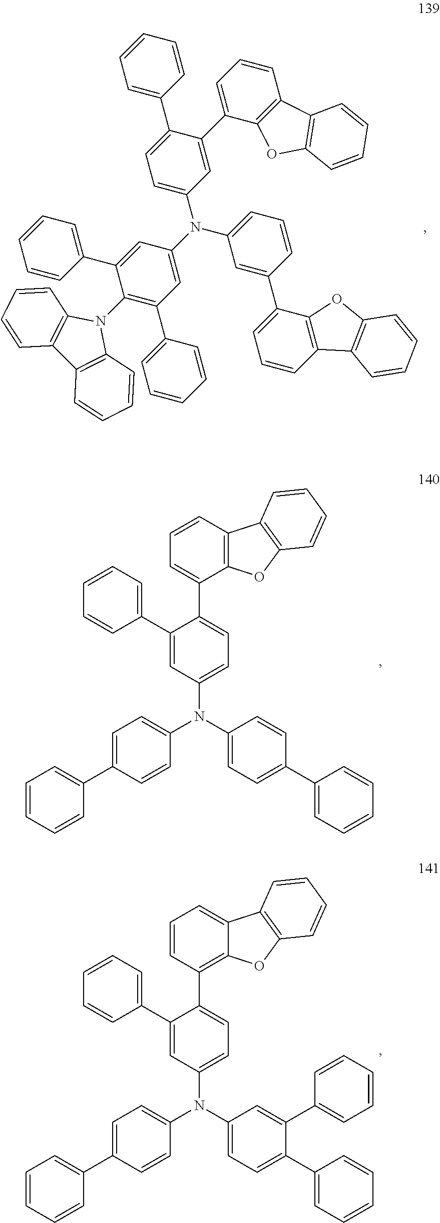 [SMILES: c1ccc(-c2ccc(N(c3cccc(-c4cccc5c4oc4ccccc45)c3)c3cc(-c4ccccc4)c(-n4c5ccccc5c5ccccc54)c(-c4ccccc4)c3)cc2-c2cccc3c2oc2ccccc23)cc1 ; c1ccc(-c2ccc(N(c3ccc(-c4ccccc4)cc3)c3ccc(-c4cccc5c4oc4ccccc45)c(-c4ccccc4)c3)cc2)cc1 ; c1ccc(-c2ccc(N(c3ccc(-c4ccccc4)c(-c4ccccc4)c3)c3ccc(-c4cccc5c4oc4ccccc45)c(-c4ccccc4)c3)cc2)cc1]

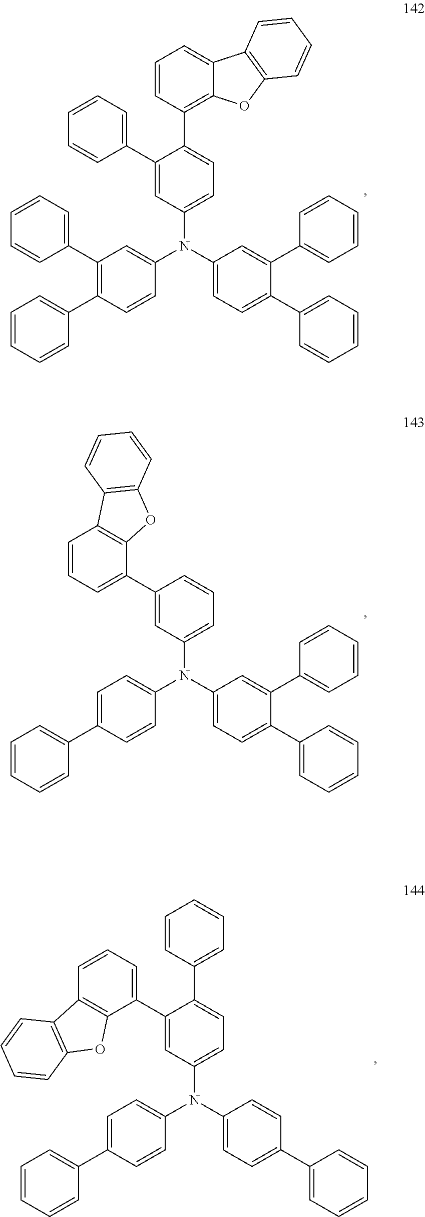 [SMILES: c1ccc(-c2ccc(N(c3ccc(-c4ccccc4)c(-c4ccccc4)c3)c3ccc(-c4cccc5c4oc4ccccc45)c(-c4ccccc4)c3)cc2-c2ccccc2)cc1 ; c1ccc(-c2ccc(N(c3cccc(-c4cccc5c4oc4ccccc45)c3)c3ccc(-c4ccccc4)c(-c4ccccc4)c3)cc2)cc1 ; c1ccc(-c2ccc(N(c3ccc(-c4ccccc4)cc3)c3ccc(-c4ccccc4)c(-c4cccc5c4oc4ccccc45)c3)cc2)cc1]

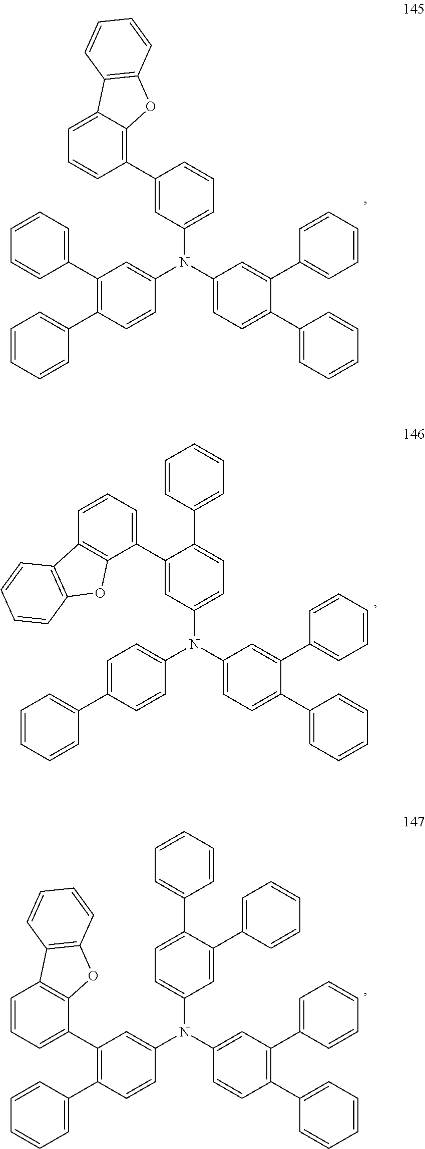 [SMILES: c1ccc(-c2ccc(N(c3cccc(-c4cccc5c4oc4ccccc45)c3)c3ccc(-c4ccccc4)c(-c4ccccc4)c3)cc2-c2ccccc2)cc1 ; c1ccc(-c2ccc(N(c3ccc(-c4ccccc4)c(-c4ccccc4)c3)c3ccc(-c4ccccc4)c(-c4cccc5c4oc4ccccc45)c3)cc2)cc1 ; c1ccc(-c2ccc(N(c3ccc(-c4ccccc4)c(-c4ccccc4)c3)c3ccc(-c4ccccc4)c(-c4cccc5c4oc4ccccc45)c3)cc2-c2ccccc2)cc1]

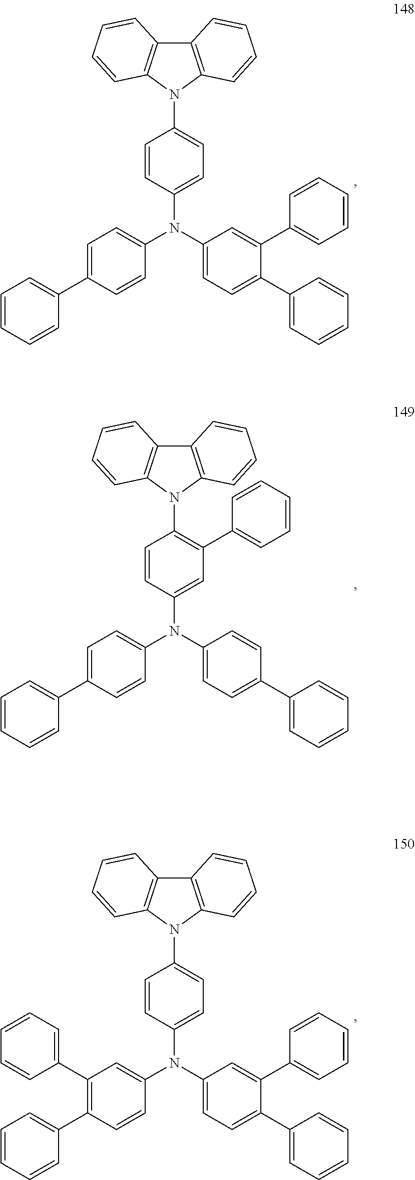 [SMILES: c1ccc(-c2ccc(N(c3ccc(-n4c5ccccc5c5ccccc54)cc3)c3ccc(-c4ccccc4)c(-c4ccccc4)c3)cc2)cc1 ; c1ccc(-c2ccc(N(c3ccc(-c4ccccc4)cc3)c3ccc(-n4c5ccccc5c5ccccc54)c(-c4ccccc4)c3)cc2)cc1 ; c1ccc(-c2ccc(N(c3ccc(-n4c5ccccc5c5ccccc54)cc3)c3ccc(-c4ccccc4)c(-c4ccccc4)c3)cc2-c2ccccc2)cc1]

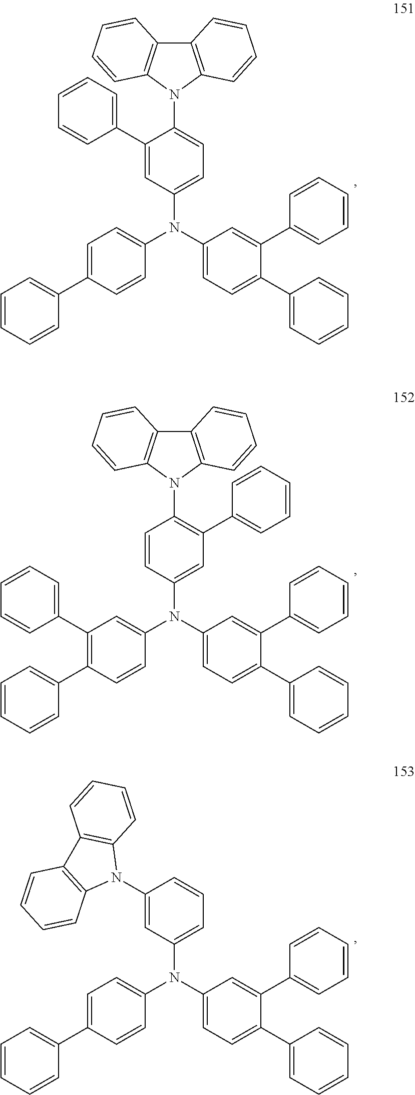 [SMILES: c1ccc(-c2ccc(N(c3ccc(-c4ccccc4)c(-c4ccccc4)c3)c3ccc(-n4c5ccccc5c5ccccc54)c(-c4ccccc4)c3)cc2)cc1 ; c1ccc(-c2ccc(N(c3ccc(-c4ccccc4)c(-c4ccccc4)c3)c3ccc(-n4c5ccccc5c5ccccc54)c(-c4ccccc4)c3)cc2-c2ccccc2)cc1 ; c1ccc(-c2ccc(N(c3cccc(-n4c5ccccc5c5ccccc54)c3)c3ccc(-c4ccccc4)c(-c4ccccc4)c3)cc2)cc1]

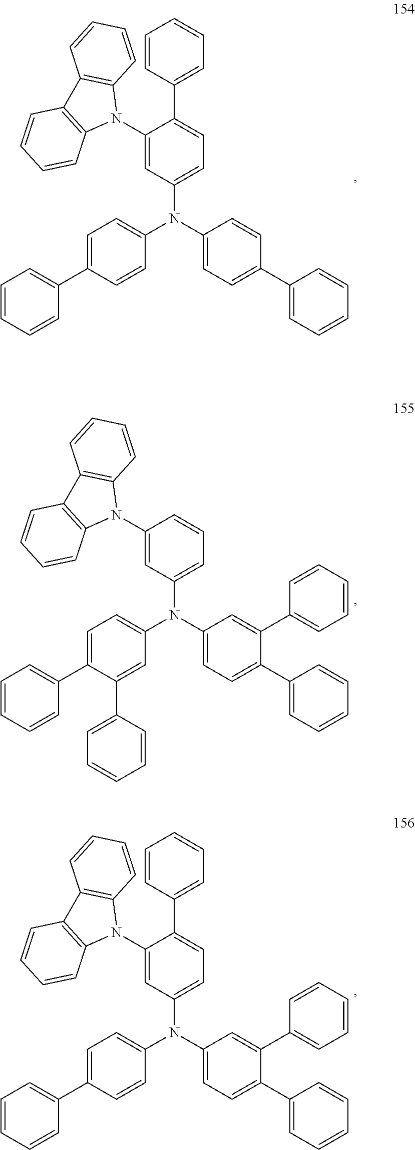 [SMILES: c1ccc(-c2ccc(N(c3ccc(-c4ccccc4)cc3)c3ccc(-c4ccccc4)c(-n4c5ccccc5c5ccccc54)c3)cc2)cc1 ; c1ccc(-c2ccc(N(c3cccc(-n4c5ccccc5c5ccccc54)c3)c3ccc(-c4ccccc4)c(-c4ccccc4)c3)cc2-c2ccccc2)cc1 ; c1ccc(-c2ccc(N(c3ccc(-c4ccccc4)c(-c4ccccc4)c3)c3ccc(-c4ccccc4)c(-n4c5ccccc5c5ccccc54)c3)cc2)cc1]

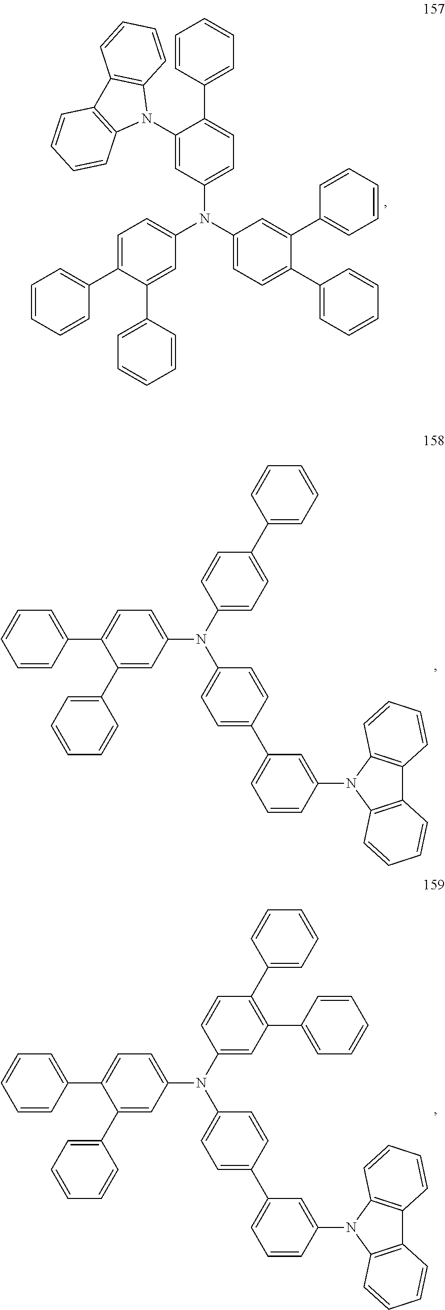 [SMILES: c1ccc(-c2ccc(N(c3ccc(-c4ccccc4)c(-c4ccccc4)c3)c3ccc(-c4ccccc4)c(-n4c5ccccc5c5ccccc54)c3)cc2-c2ccccc2)cc1 ; c1ccc(-c2ccc(N(c3ccc(-c4cccc(-n5c6ccccc6c6ccccc65)c4)cc3)c3ccc(-c4ccccc4)c(-c4ccccc4)c3)cc2)cc1 ; c1ccc(-c2ccc(N(c3ccc(-c4cccc(-n5c6ccccc6c6ccccc65)c4)cc3)c3ccc(-c4ccccc4)c(-c4ccccc4)c3)cc2-c2ccccc2)cc1]

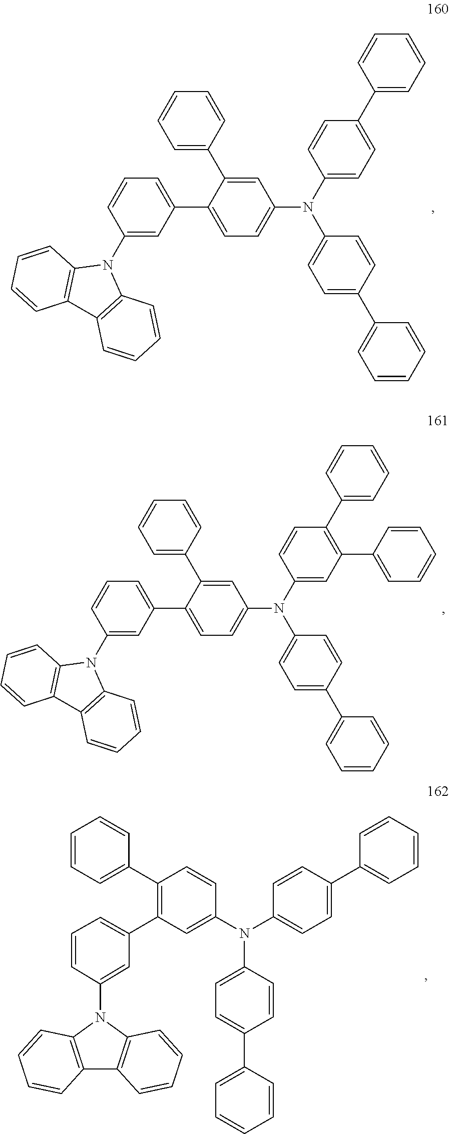 [SMILES: c1ccc(-c2ccc(N(c3ccc(-c4ccccc4)cc3)c3ccc(-c4cccc(-n5c6ccccc6c6ccccc65)c4)c(-c4ccccc4)c3)cc2)cc1 ; c1ccc(-c2ccc(N(c3ccc(-c4ccccc4)c(-c4ccccc4)c3)c3ccc(-c4cccc(-n5c6ccccc6c6ccccc65)c4)c(-c4ccccc4)c3)cc2)cc1 ; c1ccc(-c2ccc(N(c3ccc(-c4ccccc4)cc3)c3ccc(-c4ccccc4)c(-c4cccc(-n5c6ccccc6c6ccccc65)c4)c3)cc2)cc1]

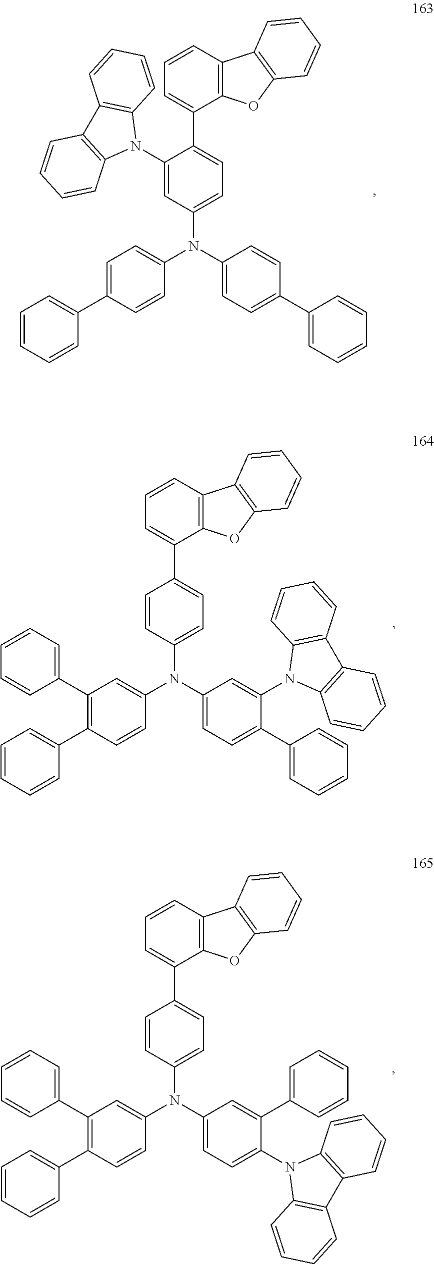 [SMILES: c1ccc(-c2ccc(N(c3ccc(-c4ccccc4)cc3)c3ccc(-c4cccc5c4oc4ccccc45)c(-n4c5ccccc5c5ccccc54)c3)cc2)cc1 ; c1ccc(-c2ccc(N(c3ccc(-c4cccc5c4oc4ccccc45)cc3)c3ccc(-c4ccccc4)c(-n4c5ccccc5c5ccccc54)c3)cc2-c2ccccc2)cc1 ; c1ccc(-c2ccc(N(c3ccc(-c4cccc5c4oc4ccccc45)cc3)c3ccc(-n4c5ccccc5c5ccccc54)c(-c4ccccc4)c3)cc2-c2ccccc2)cc1]

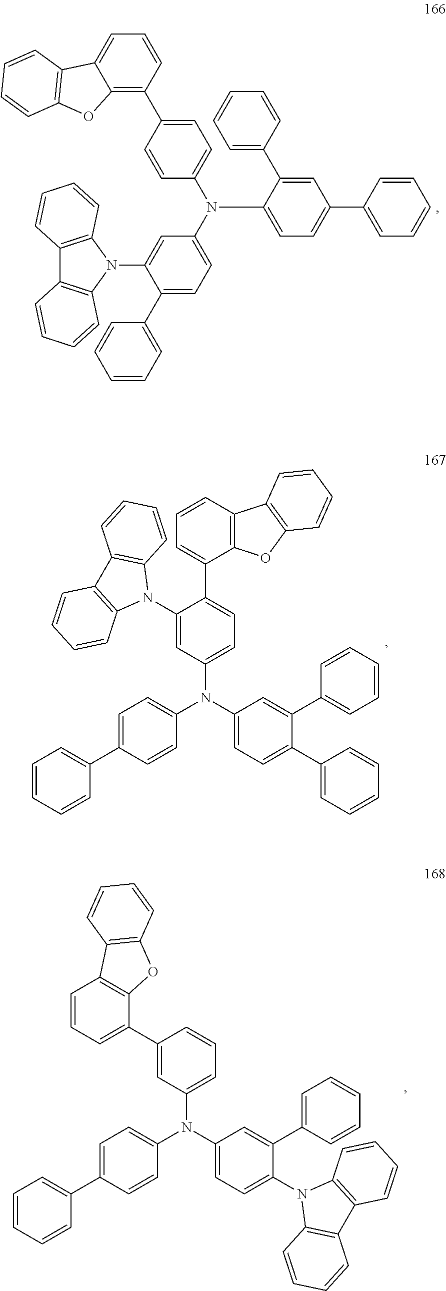 [SMILES: c1ccc(-c2ccc(N(c3ccc(-c4cccc5c4oc4ccccc45)cc3)c3ccc(-c4ccccc4)c(-n4c5ccccc5c5ccccc54)c3)c(-c3ccccc3)c2)cc1 ; c1ccc(-c2ccc(N(c3ccc(-c4ccccc4)c(-c4ccccc4)c3)c3ccc(-c4cccc5c4oc4ccccc45)c(-n4c5ccccc5c5ccccc54)c3)cc2)cc1 ; c1ccc(-c2ccc(N(c3cccc(-c4cccc5c4oc4ccccc45)c3)c3ccc(-n4c5ccccc5c5ccccc54)c(-c4ccccc4)c3)cc2)cc1]

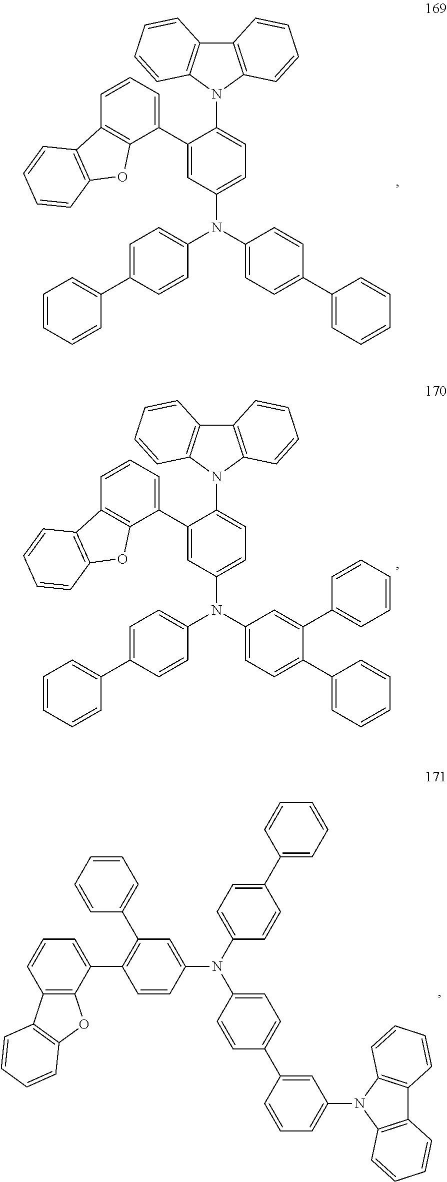 [SMILES: c1ccc(-c2ccc(N(c3ccc(-c4ccccc4)cc3)c3ccc(-n4c5ccccc5c5ccccc54)c(-c4cccc5c4oc4ccccc45)c3)cc2)cc1 ; c1ccc(-c2ccc(N(c3ccc(-c4ccccc4)c(-c4ccccc4)c3)c3ccc(-n4c5ccccc5c5ccccc54)c(-c4cccc5c4oc4ccccc45)c3)cc2)cc1 ; c1ccc(-c2ccc(N(c3ccc(-c4cccc(-n5c6ccccc6c6ccccc65)c4)cc3)c3ccc(-c4cccc5c4oc4ccccc45)c(-c4ccccc4)c3)cc2)cc1]

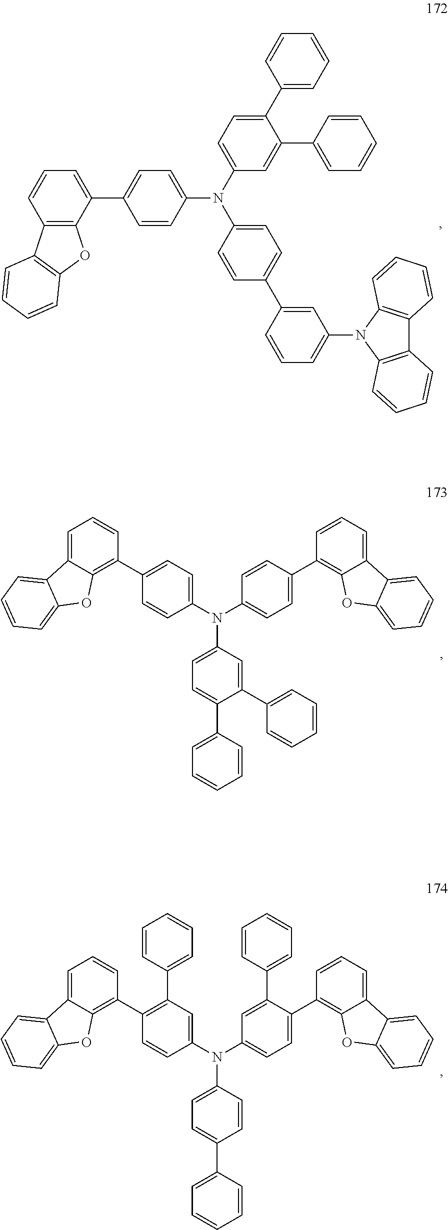 [SMILES: c1ccc(-c2ccc(N(c3ccc(-c4cccc(-n5c6ccccc6c6ccccc65)c4)cc3)c3ccc(-c4cccc5c4oc4ccccc45)cc3)cc2-c2ccccc2)cc1 ; c1ccc(-c2ccc(N(c3ccc(-c4cccc5c4oc4ccccc45)cc3)c3ccc(-c4cccc5c4oc4ccccc45)cc3)cc2-c2ccccc2)cc1 ; c1ccc(-c2ccc(N(c3ccc(-c4cccc5c4oc4ccccc45)c(-c4ccccc4)c3)c3ccc(-c4cccc5c4oc4ccccc45)c(-c4ccccc4)c3)cc2)cc1]

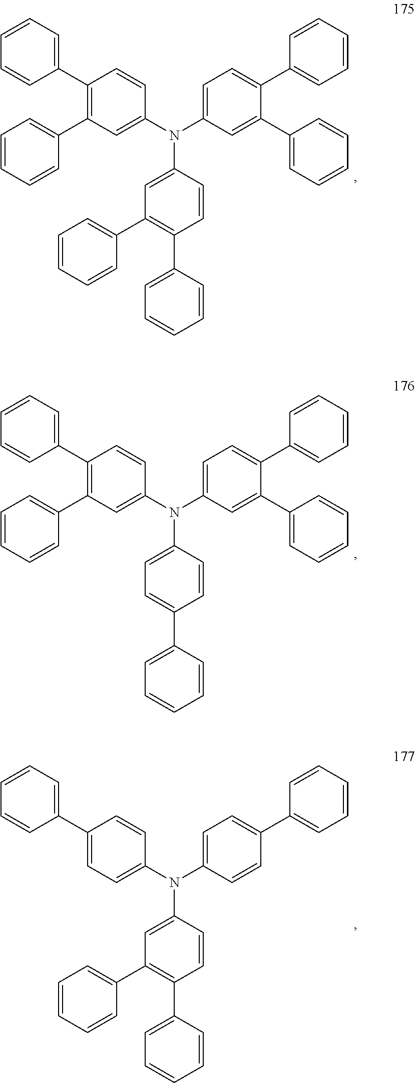 [SMILES: c1ccc(-c2ccc(N(c3ccc(-c4ccccc4)c(-c4ccccc4)c3)c3ccc(-c4ccccc4)c(-c4ccccc4)c3)cc2-c2ccccc2)cc1 ; c1ccc(-c2ccc(N(c3ccc(-c4ccccc4)c(-c4ccccc4)c3)c3ccc(-c4ccccc4)c(-c4ccccc4)c3)cc2)cc1 ; c1ccc(-c2ccc(N(c3ccc(-c4ccccc4)cc3)c3ccc(-c4ccccc4)c(-c4ccccc4)c3)cc2)cc1]

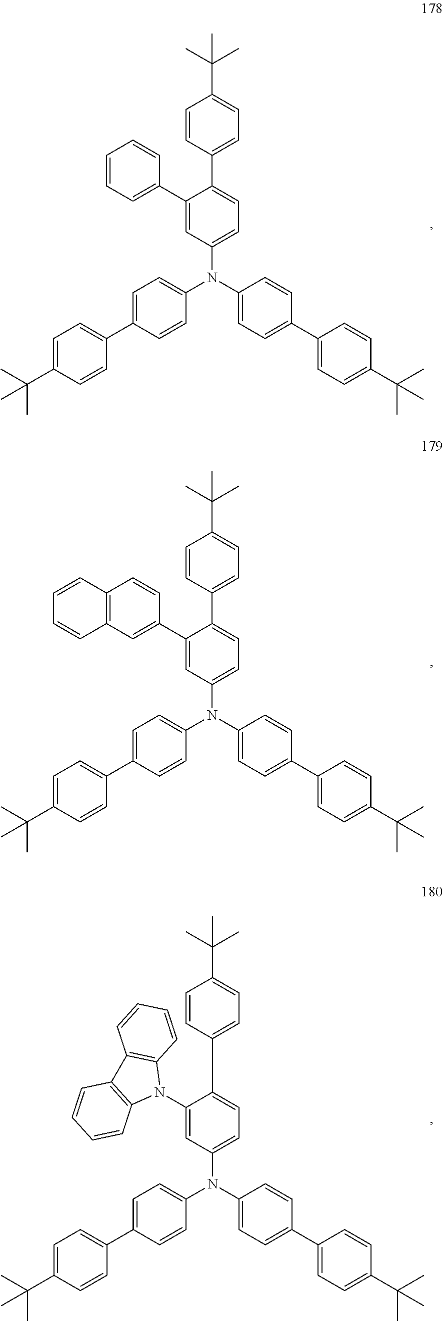

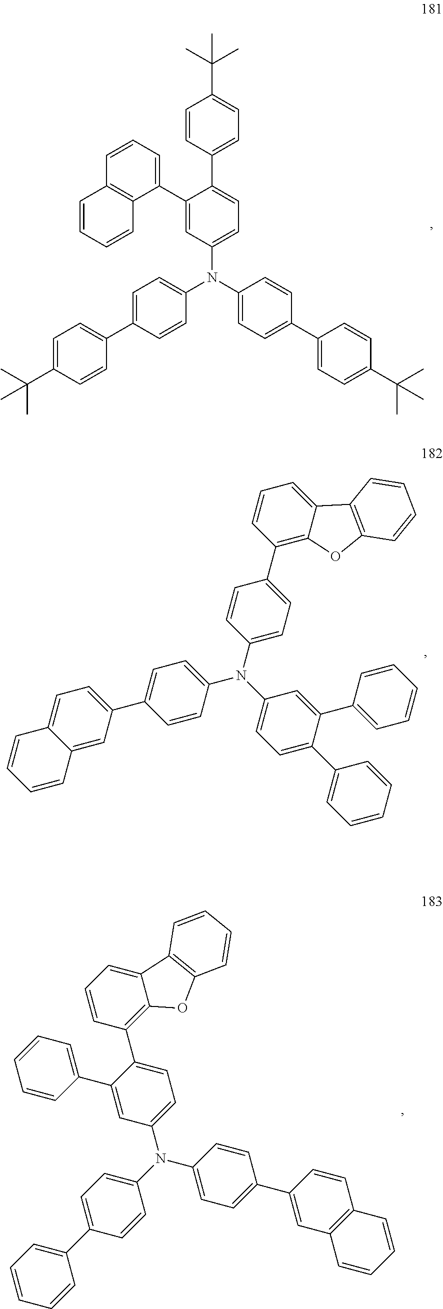 [SMILES: CC(C)(C)c1ccc(-c2ccc(N(c3ccc(-c4ccc(C(C)(C)C)cc4)cc3)c3ccc(-c4ccc(C(C)(C)C)cc4)c(-c4cccc5ccccc45)c3)cc2)cc1 ; c1ccc(-c2ccc(N(c3ccc(-c4ccc5ccccc5c4)cc3)c3ccc(-c4cccc5c4oc4ccccc45)cc3)cc2-c2ccccc2)cc1 ; c1ccc(-c2ccc(N(c3ccc(-c4ccc5ccccc5c4)cc3)c3ccc(-c4cccc5c4oc4ccccc45)c(-c4ccccc4)c3)cc2)cc1]

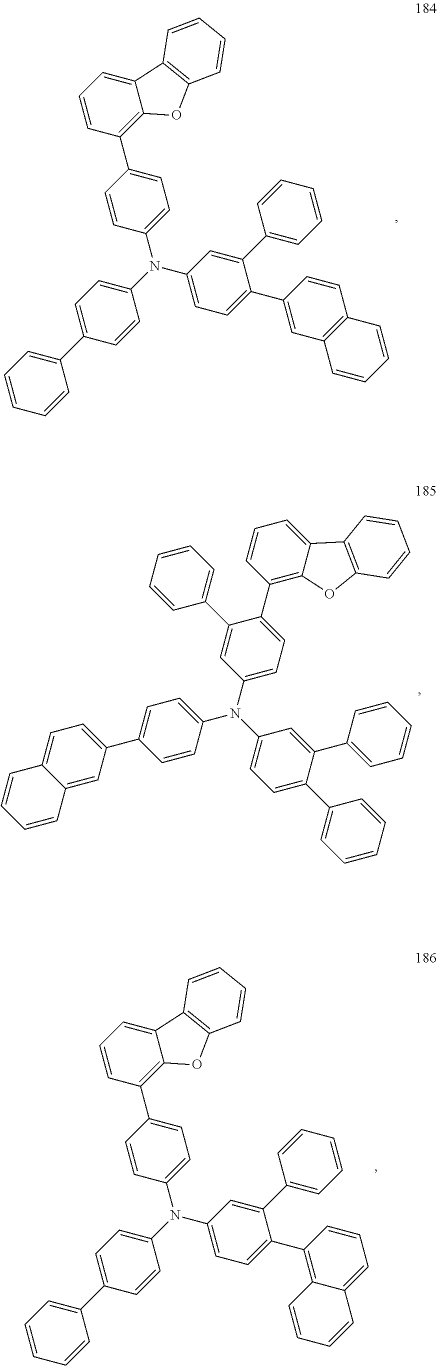 [SMILES: c1ccc(-c2ccc(N(c3ccc(-c4cccc5c4oc4ccccc45)cc3)c3ccc(-c4ccc5ccccc5c4)c(-c4ccccc4)c3)cc2)cc1 ; c1ccc(-c2ccc(N(c3ccc(-c4ccc5ccccc5c4)cc3)c3ccc(-c4cccc5c4oc4ccccc45)c(-c4ccccc4)c3)cc2-c2ccccc2)cc1 ; c1ccc(-c2ccc(N(c3ccc(-c4cccc5c4oc4ccccc45)cc3)c3ccc(-c4cccc5ccccc45)c(-c4ccccc4)c3)cc2)cc1]

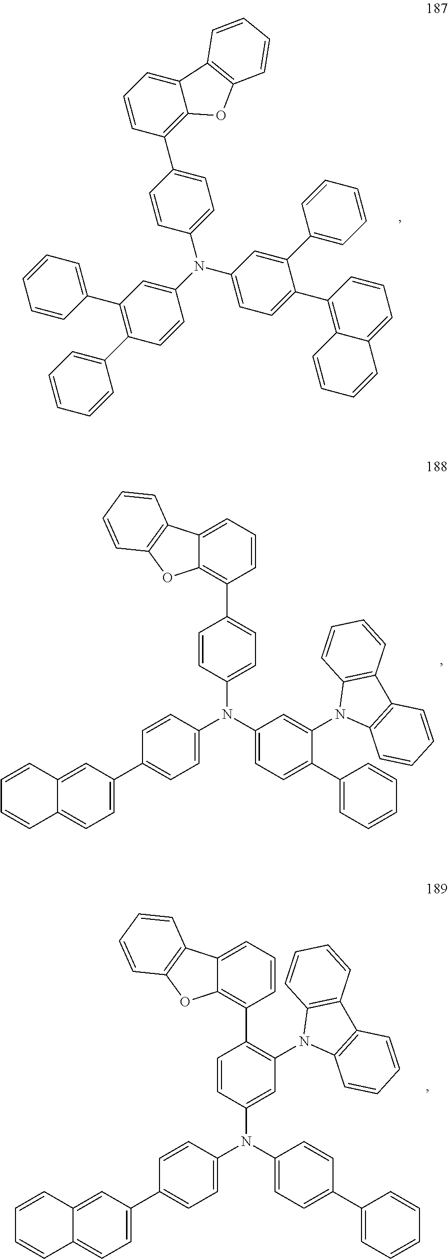 [SMILES: c1ccc(-c2ccc(N(c3ccc(-c4cccc5c4oc4ccccc45)cc3)c3ccc(-c4cccc5ccccc45)c(-c4ccccc4)c3)cc2-c2ccccc2)cc1 ; c1ccc(-c2ccc(N(c3ccc(-c4ccc5ccccc5c4)cc3)c3ccc(-c4cccc5c4oc4ccccc45)cc3)cc2-n2c3ccccc3c3ccccc32)cc1 ; c1ccc(-c2ccc(N(c3ccc(-c4ccc5ccccc5c4)cc3)c3ccc(-c4cccc5c4oc4ccccc45)c(-n4c5ccccc5c5ccccc54)c3)cc2)cc1]

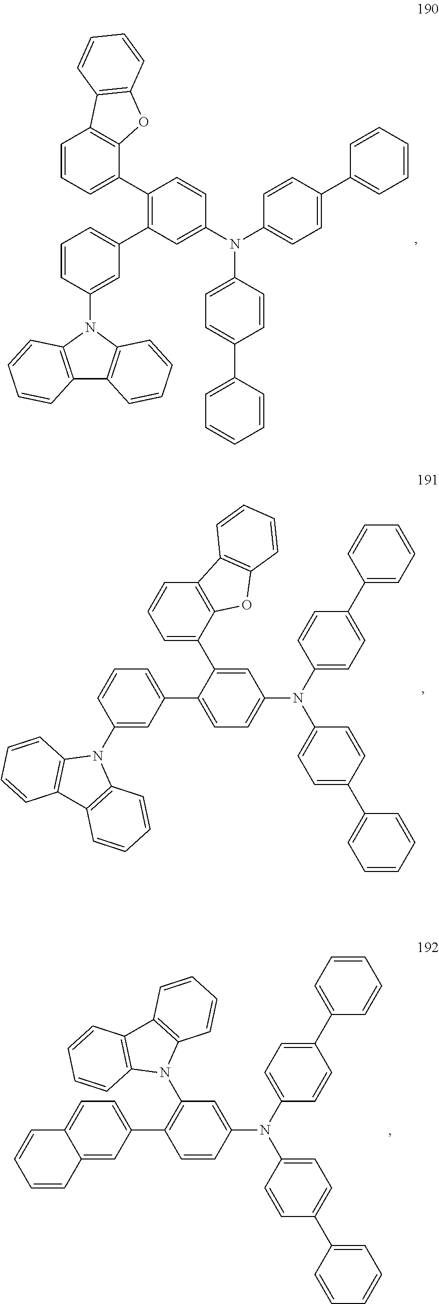 [SMILES: c1ccc(-c2ccc(N(c3ccc(-c4ccccc4)cc3)c3ccc(-c4cccc5c4oc4ccccc45)c(-c4cccc(-n5c6ccccc6c6ccccc65)c4)c3)cc2)cc1 ; c1ccc(-c2ccc(N(c3ccc(-c4ccccc4)cc3)c3ccc(-c4cccc(-n5c6ccccc6c6ccccc65)c4)c(-c4cccc5c4oc4ccccc45)c3)cc2)cc1 ; c1ccc(-c2ccc(N(c3ccc(-c4ccccc4)cc3)c3ccc(-c4ccc5ccccc5c4)c(-n4c5ccccc5c5ccccc54)c3)cc2)cc1]

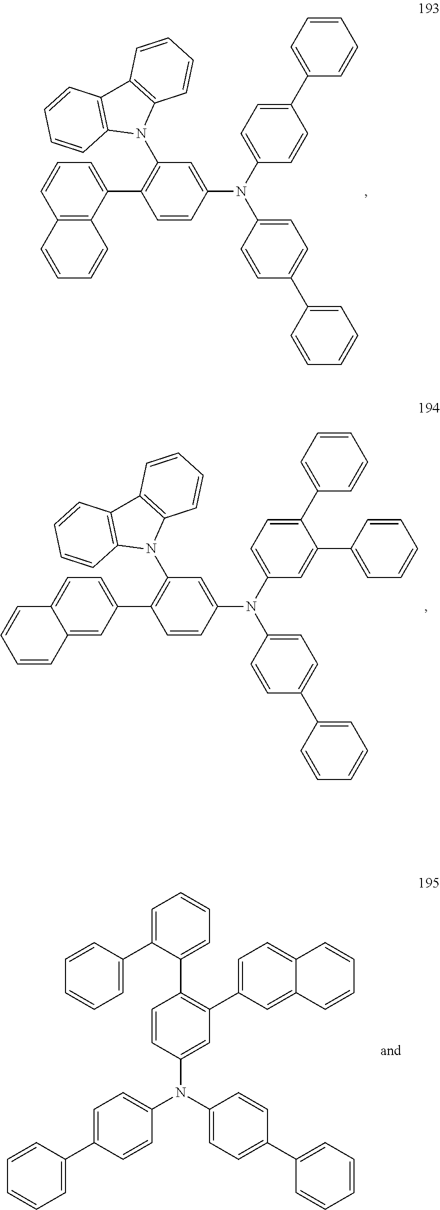 [SMILES: c1ccc(-c2ccc(N(c3ccc(-c4ccccc4)cc3)c3ccc(-c4cccc5ccccc45)c(-n4c5ccccc5c5ccccc54)c3)cc2)cc1 ; c1ccc(-c2ccc(N(c3ccc(-c4ccccc4)c(-c4ccccc4)c3)c3ccc(-c4ccc5ccccc5c4)c(-n4c5ccccc5c5ccccc54)c3)cc2)cc1 ; c1ccc(-c2ccc(N(c3ccc(-c4ccccc4)cc3)c3ccc(-c4ccccc4-c4ccccc4)c(-c4ccc5ccccc5c4)c3)cc2)cc1]

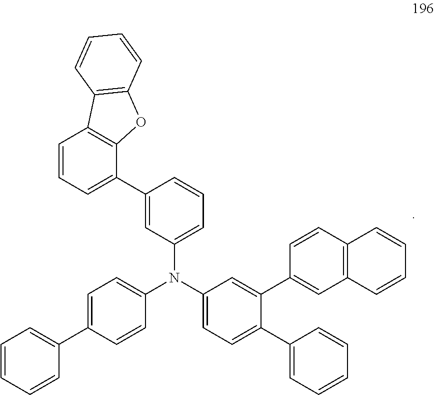 [SMILES: c1ccc(-c2ccc(N(c3cccc(-c4cccc5c4oc4ccccc45)c3)c3ccc(-c4ccccc4)c(-c4ccc5ccccc5c4)c3)cc2)cc1]

6. A method for preparing the compound according to claim 1, characterized in that the method involves the following reaction schemes: 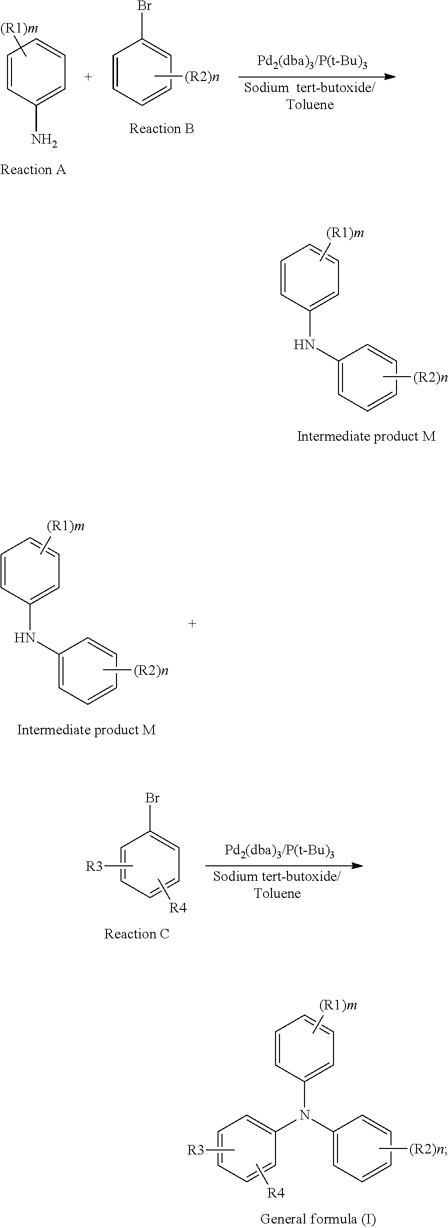
the method specifically comprises the following steps:
(1) With reactant A and reactant B as raw materials and toluene as solvent, Pd₂(dba)₃, P(t-Bu)₃ and sodium tert-butoxide are added to the reaction system under a nitrogen atmosphere, reacted at 95° C. to 110° C. for 10 to 24 h, and naturally cooled to room temperature; and the reaction solution is filtered, and the filtrate is subjected to rotary evaporation under reduced pressure and passed through a neutral silica gel column to obtain intermediate product M, wherein the toluene is used in an amount of 50 to 80 ml per g of the reactant A; the reactant A and the reactant B are present in a molar ratio of 1:0.8 to 1; Pd₂(dba)₃ and the reactant A are present in a molar ratio of 0.005 to 0.01:1; P(t-Bu)₃ and the reactant A are present in a molar ratio of 1.5 to 3.0:1; and sodium tert-butoxide and the reactant A are present in a molar ratio of 2 to 2.5:1;
(2) With the intermediate product M obtained in step (1) and reactant C as raw materials and toluene as solvent, Pd₂(dba)₃, P(t-Bu)₃ and sodium tert-butoxide are added to the reaction system under a nitrogen atmosphere, reacted at 95° C. to 110° C. for 10 to 24 h, and naturally cooled to room temperature; and the reaction solution is filtered, and the filtrate is subjected to rotary evaporation under reduced pressure and passed through a neutral silica gel column to obtain a compound of general formula (1), wherein the toluene is used in an amount of 50 to 80 ml per g of the intermediate product M; the intermediate product M and the reactant C are present in a molar ratio of 1:1.0 to 1.5; the Pd₂(dba)₃ and the intermediate product M are present in a molar ratio of 0.005 to 0.01:1; the P(t-Bu)₃ and the intermediate product M are present in a molar ratio of 1.5 to 3.0:1; and the sodium tert-butoxide and the intermediate product M are present in a molar ratio of 2 to 2.5:1.

7. An organic light-emitting device, characterized in that the organic light-emitting device contains at least one functional layer comprising the compound with a core structure of triarylamine according to claim 1.

8. The organic light-emitting device according to claim 7, characterized in that the compound with a core structure of triarylamine is used as a hole transporting layer or an electron blocking layer materials for making the organic light-emitting device.

9. A lighting or display element, characterized in that the element comprises the organic light-emitting device according to claim 7.
